(19) United States
(12) Patent Application Publication
(10) Pub. No.: US 2021/0236512 A1
(43) Pub. Date: Aug. 5, 2021

(54) CLOZAPINE FOR THE TREATMENT OF A IMMUNOGLOBULIN DRIVEN B CELL DISEASE

(71) Applicant: ZARODEX THERAPEUTICS LIMITED, London (GB)

(72) Inventors: Stephen JOLLES, Cardiff (GB); Houman ASHRAFIAN, London (GB); Duncan MCHALE, London (GB)

(73) Assignee: ZARODEX THERAPEUTICS LIMITED, London (GB)

(21) Appl. No.: 16/965,244

(22) PCT Filed: Jan. 31, 2019
(86) PCT No.: PCT/EP2019/052450

§ 371 Date: Jul. 27, 2020

(30) Foreign Application Priority Data
Jan. 31, 2018 (EP) 18154552.6

(51) Int. Cl. A61K 31/5513; A61K 9/00; A61K 39/395; A61K 31/436; A61K 38/13; A61K 31/5377; A61K 31/573; A61K 31/519; A61K 31/675; A61K 31/4706; A61K 31/52; A61K 31/192; A61P 19/02; A61P 37/06

(52) U.S. Cl. CPC A61K 31/5513; A61K 9/0056; A61K 39/3955; A61K 31/436; A61K 38/13; A61K 31/5377; A61P 37/06; A61K 31/519; A61K 31/675; A61K 31/4706; A61K 31/52; A61K 31/192; A61P 19/02; A61K 31/573

(57) ABSTRACT

This invention relates to the compound clozapine and its major metabolite norclozapine and prodrugs thereof and pharmaceutically acceptable salts and solvates thereof for use in the treatment or prevention of a pathogenic immunoglobulin driven B cell disease with a T cell component. The invention also provides pharmaceutical compositions containing such compounds.

### TECHNICAL FIELD

This invention relates to a compound and pharmaceutical compositions containing such compound for use in the treatment or prevention of a pathogenic immunoglobulin driven B cell disease with a T cell component.

### BACKGROUND TO THE INVENTION

The compound associated with this invention is known as clozapine i.e. the compound of the following structure:

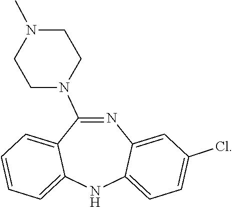 [SMILES: CN1CCN(C2=Nc3cc(Cl)ccc3Nc3ccccc32)CC1]

Clozapine has a major active metabolite known as norclozapine (Guitton et al., 1999) which has the following structure:

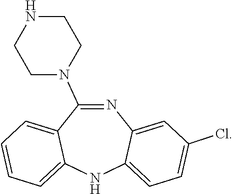 [SMILES: Clc1ccc2c(c1)N=C(N1CCNCC1)c1ccccc1N2]

Clozapine is known as a treatment for resistant schizophrenia. Schizophrenia is an enduring major psychiatric disorder affecting around 1% of the population. Apart from the debilitating psychiatric symptoms it has serious psychosocial consequences with an unemployment rate of 80-90% and a life expectancy reduced by 10-20 years. The rate of suicide among people with schizophrenia is much higher than in the general population and approximately 5% of those diagnosed with schizophrenia commit suicide.

Clozapine is an important therapeutic agent and is included on the WHO list of essential medicines. It is a dibenzo-diazepine atypical antipsychotic, and since 1990 the only licensed therapy in the UK for the 30% of patients with treatment-resistant schizophrenia (TRS). It shows superior efficacy in reducing both positive and negative symptoms in schizophrenic patients and is effective in approximately 60% of previously treatment refractive patients with a significant reduction in suicide risk. The National Institute for Health and Clinical Excellence (NICE) guideline recommends adults with schizophrenia which has not responded adequately to treatment with at least 2 antipsychotic drugs (at least one of which should be a non-clozapine second generation antipsychotic) should be offered clozapine.

Clozapine is associated with serious adverse effects including seizures, intestinal obstruction, diabetes, thromboembolism, cardiomyopathy and sudden cardiac death. It can also cause agranulocytosis (cumulative incidence 0.8%); necessitating intensive centralised registry based monitoring systems to support its safe use. In the UK there are three electronic registries (www.clozaril.co.uk, www.denzapine.co.uk and www.ztas.co.uk) one for each of the clozapine suppliers. Mandatory blood testing is required weekly for the first 18 weeks, then every two weeks from weeks 19-52 and thereafter monthly with a ‘red flag’ cut-off value for absolute neutrophil count (ANC) of less than 1500/4 for treatment interruption.

In 2015, the Federal Drug Administration (FDA) merged and replaced the six existing clozapine registries in the United States combining data from over 50,000 prescribers, 28,000 pharmacies and 90,000 patients records into a single shared registry for all clozapine products, the Clozapine Risk Evaluation and Mitigation Strategy (REMS) Program (www.clozapinerems.com). Changes were introduced lowering the absolute neutrophil count (ANC) threshold to interrupt clozapine treatment at less than 1000/4 in general, and at less than 500/4 in benign ethnic neutropenia (BEN). Prescribers have greater flexibility to make patient-specific decisions about continuing or resuming treatment in patients who develop moderate to severe neutropenia, and so maximize patient benefit from access to clozapine.

Schizophrenia is associated with a 3.5 fold increased chance of early death compared to the general population. This is often due to physical illness, in particular chronic obstructive pulmonary disease (COPD) (Standardised Mortality Ratio (SMR) 9.9), influenza and pneumonia (SMR 7.0). Although clozapine reduces overall mortality in severe schizophrenia, there is a growing body of evidence linking clozapine with elevated rates of pneumonia-related admission and mortality. In an analysis of 33,024 patients with schizophrenia, the association between second generation antipsychotic medications and risk of pneumonia requiring hospitalization was highest for clozapine with an adjusted risk ratio of 3.18 with a further significant increase in risk associated with dual antipsychotic use (Kuo et al., 2013). Although quetiapine, olanzapine, zotepine, and risperidone were associated with a modestly increased risk, there was no clear dose-dependent relationship and the risk was not significant at time points beyond 30 days (Leung et al., 2017; Stoecker et al., 2017).

In a 12 year study of patients taking clozapine, 104 patients had 248 hospital admissions during the study period. The predominant admission types were for treatment of either pulmonary (32.2%) or gastrointestinal (19.8%) illnesses. The commonest pulmonary diagnosis was pneumonia, (58% of pulmonary admissions) and these admissions were unrelated to boxed warnings (Leung et al., 2017).

In a further nested case control study clozapine was found to be the only antipsychotic with a clear dose-dependent risk for recurrent pneumonia, this risk increased on re-exposure to clozapine (Hung et al., 2016).

While these studies underscore the increased admissions or deaths from pneumonia and sepsis in patients taking clozapine over other antipsychotics, the focus on extreme outcomes (death and pneumonia) may underestimate the burden of less severe but more frequent infections such as sinusitis, skin, eye, ear or throat infections and community acquired and treated pneumonia. Infection may represent an important additional factor in destabilizing schizophrenia control and clozapine levels.

Various mechanisms for the increase in pneumonia have been suggested, including aspiration, sialorrhoea and impairment of swallowing function with oesophageal dilatation, hypomotility and agranulocytosis. In addition, cigarette smoking is highly prevalent among patients with schizophrenia as a whole and represents an independent risk factor for pneumonia incidence and severity.

A small amount of research into the immunomodulatory properties of clozapine has been performed:

Hinze-Selch et al (Hinze-Selch et al., 1998) describes clozapine as an atypical antipsychotic agent with immunomodulatory properties. This paper reports that patients that received clozapine treatment for six weeks showed significant increases in the serum concentrations of IgG, but no significant effect was found on IgA or IgM concentrations or on the pattern of autoantibodies.

Jolles et al (Jolles et al., 2014) reports studies on the parameter “calculated globulin (CG)” as a screening test for antibody deficiency. Patients with a wide range of backgrounds were selected from thirteen laboratories across Wales. Of the patients with significant antibody deficiency (IgG <4 g/L, reference range 6-16 g/L), identified on CG screening from primary care, clozapine use was mentioned on the request form in 13% of the samples. However, antibody deficiency is not a listed side effect of clozapine in the British National Formulary (BNF), nor does antibody testing constitute part of current clozapine monitoring protocols.

Another study by Lozano et al. (Lozano et al., 2016) reported an overall decrease of mean plasma levels of IgM in the study group (which consisted of psychiatric outpatients who took clozapine for at least five years) compared to the control group, and also reported that no differences were found between the groups with respect to IgA, IgG, absolute neutrophil count and white blood cell count.

Consequently, given these mixed results that have been reported, the immunomodulatory properties of clozapine and its effect on immunoglobin levels are neither clear nor understood in the art.

Pathogenic immunoglobulin (including IgG, IgA and IgM) driven B cell diseases with a T cell component result from secretion of autoantibodies (principally IgG and IgA) by antibody secreting cells (ASCs, collectively plasmablasts and plasma cells, these being types of mature B cell). These antibodies target a variety of self-antigens (in the case of IgG and IgA driven diseases) which have been characterised in some of these conditions. There is rarely an increase in overall immunoglobulins as the pathological process is driven by the secretion of specific immunoglobulins which constitute a small percentage of the total immunoglobulins. Secretion of IgG and IgA antibodies is from ASCs, and ASCs are generated secondary to the differentiation of class-switched and unswitched memory B cells, these being further types of mature B cell. Various lines of evidence suggest this is a highly-dynamic process, with ongoing differentiation occurring almost constantly. The T cell component that contributes towards the pathology of the diseases arises because B cells act as professional antigen-presenting cells for T cells (their importance is increased also due to their sheer numbers). B cells secrete significant amounts of cytokines that impact T cells and B-T cell interaction is involved in responses to T dependent protein antigens and class switching. T cells will therefore contribute in a number of ways in the activity and the maturation of the B-cells.

Class-switched memory B cells are mature B cells that have replaced their primary encoded membrane receptor [IgM] by IgG, IgA or IgE in response to repeated antigen recognition. This class-switching process is a key feature of normal humoral immunological memory, both ‘constitutive’ through the secretion of pre-existing protective antibodies by long-lived plasma cells, and ‘reactive’ reflecting re-exposure to antigen and reactivation of memory B cells to either differentiate into plasma cells to produce antibodies, or to germinal centre B cells to enable further diversification and affinity maturation of the antibody response. Early in the immune response, plasma cells derive from unswitched activated B cells and secrete IgM. Later in the immune response, plasma cells originate from activated B cells participating in the germinal centre (areas forming in secondary lymphoid follicular tissue in response to antigenic challenge) which have undergone class switching (retaining antigen specificity but exchanging immunoglobulin isotype) and B cell receptor (BCR) diversification through immunoglobulin somatic hypermutation. This maturation process enables the generation of BCRs with high affinity to antigen and production of different immunoglobulin isotypes (i.e. exchanging the originally expressed IgM and IgD to IgG, IgA or IgE isotypes) (Budeus et al., 2015; Kracker and Durandy, 2011).

Class switch recombination (CSR) following the germinal centre reaction in secondary lymphoid organs provides antigen-primed/experienced autoreactive memory B cells and a core pathway for development and/or maintenance of autoimmunity. Post-germinal centre B cells class-switched to IgG or IgA in the periphery can also enter other anatomic compartments, such as the central nervous system, to undergo further affinity maturation (e.g. in tertiary lymphoid structures in multiple sclerosis) and contribute to immune pathology (Palanichamy et al., 2014). CSR can also occur locally within tissue in pathology, such as within ectopic lymphoid structures in chronically inflamed tissue such as rheumatoid arthritis synovium (Alsaleh et al., 2011; Humby et al., 2009).

A significant proportion of bone marrow plasma cells are IgA⁺ (^{˜}40%) with IgA⁺ plasma cells further constituting the majority in serum (^{˜}80%) (Mei et al., 2009) consistent with a substantial contribution of IgA⁺ plasma cells to the bone marrow population of long-lived cells. The intestinal mucosa is the primary inductive site for IgA⁺ plasma cells, mainly through gut-associated lymphoid tissue (GALT, comprising Peyer's patches and isolated lymphoid follicles) (Craig and Cebra, 1971), together with mesenteric lymph nodes and, potentially, the intestinal lamina propria itself, with class-switch recombination towards IgA achieved through both T cell-independent (pre-germinal centre formation) (Bergqvist et al., 2010; Casola et al., 2004) and T cell-dependent mechanisms (Pabst, 2012). Notably, IgA⁺ and other plasma cells (in addition to plasmablasts) are increasingly understood to exert important effector immune functions beyond the production of immunoglobulin, including generation of cytokines (Shen and Fillatreau, 2015) and immunoregulators such as tumour-necrosis factor-α (TNF-α), inducible nitric oxide synthase (iNOS) (Fritz et al., 2011), IL-10 (Matsumoto et al., 2014; Rojas et al., 2019), IL-35 (Shen et al., 2014), IL-17a (Bermejo et al., 2013) and ISG15 (Care et al., 2016).

Plasmablasts, representing short-lived rapidly cycling antibody-secreting cells of the B cell lineage with migratory capacity, are also precursors to long-lived (post-mitotic) plasma cells, including those which home in to the bone marrow niche (Nutt et al., 2015). In addition to being precursors of autoreactive long-lived plasma cells, plasmablasts are an important potential therapeutic target themselves through their ability to produce pathogenic immunoglobulin/autoantibody (Hoyer et al., 2004), particularly IgG but also IgM, described in several disease contexts such as neuromyelitis optica (Chihara et al., 2013; Chihara et al., 2011), idiopathic pulmonary arterial hypertension, IgG4-related disease (Wallace et al., 2015), multiple sclerosis (Rivas et al., 2017) and transverse myelitis (Ligocki et al., 2013), rheumatoid arthritis (Owczarczyk et al., 2011) and systemic lupus erythematosus (SLE) (Banchereau et al., 2016). In addition to their direct antibody secreting function, circulating plasmablasts also exert activity to potentiate germinal centre-derived immune responses and thereby antibody production via a feed-forward mechanism involving Il-6-induced promotion of T follicular helper cell (Tfh) differentiation and expansion (Chavele et al., 2015).

Long-lived plasma cells, whose primary residency niche is in bone marrow (Benner et al., 1981), are thought to be the major source of stable autoantibody production in (both physiologic) and pathogenic states and are resistant to glucocorticoids, conventional immunosuppressive and B cell depleting therapies (Hiepe et al., 2011). Substantiating the critical importance of this B cell population to long-term antibody production, site-specific survival of bone marrow-derived plasma cells with durable (up to 10 years post-immunisation) antibody responses to prior antigens has been demonstrated in non-human primates despite sustained memory B cell depletion (Hammarlund et al., 2017). Given the key role played by autoreactive long-lived plasma cells in the maintenance of autoimmunity (Mumtaz et al., 2012)—and the substantial refractoriness of the autoreactive memory formed by these cells to conventional immunosuppressive agents such as anti-TNF or B cell depleting biologics (Hiepe et al., 2011)

CD19(+) B cells and CD19(−) B plasma cells are drivers of pathogenic immunoglobulin driven B cell diseases. Pathogenic immunoglobulin driven B cell diseases represent a substantial proportion of all autoimmune and inflammatory diseases. The most prominent, but not the sole mechanism through which pathogenic immunoglobulin driven B cells cause disease, is through auto-antibody production. Pathogenic immunoglobulin driven B cell diseases with a T cell component are poorly treated and as a result they have substantial mortality and morbidity rates, even for the “benign” diseases. Certain current advanced therapies are directed at mature B cells. For example, belimumab is a human monoclonal antibody that inhibits B cell activating factor. Atacicept is a recombinant fusion protein that also inhibits B cell activating factor. However, memory B cells may be resistant to therapies such as belimumab or atacicept which target survival signals such as B cell activation factor (Stohl et al., 2012). The importance of memory B cells in the pathogenesis of autoimmune disorders was also demonstrated by the lack of efficacy of atacicept in treating rheumatoid arthritis and multiple sclerosis (Kappos et al., 2014; Richez et al., 2014). Plasmapheresis and immunoabsorption involve the removal of disease-causing autoantibodies from the patient's bloodstream. However, these treatments have limited efficacy or are complex and costly to deliver. CAR-T methods directed at CD19(+) B cells leaves CD19(−) B plasma cells intact, which makes it ineffective. Rituximab is a drug that is currently used to treat some pathogenic IgG driven B cell diseases. It targets B cells that express CD20. However, CD20 is only expressed on a limited subset of B cells. It also does not target plasma cells. This limited expression of CD20 and lack of effect on plasma cells explains the limited efficacy of rituximab in a variety of diseases, both benign and malignant, despite being definitively of B cell origin. Rituximab does not appear to have any effect on IgA-secreting plasmablasts/plasma cells, and consequently the associated IgA driven B cell diseases (Yong et al., 2015).

Thus, there is a major unmet medical need for new treatments against pathogenic immunoglobulin driven B cell diseases with a T cell component.

### SUMMARY OF THE INVENTION

It has been found by the present inventors that clozapine treatment in humans is associated with a significant reduction in immunoglobulin levels and impaired responses to vaccination with T-independent unconjugated pneumococcal polysaccharide antigens and T-dependent protein antigens (e.g. Hib) confirming both a quantitative and qualitative impact on B cell antibody production. In addition, there is a significant reduction in levels of class switched memory B cells (CSMB) and an observed reduction in levels of plasmablasts, both types of mature B cell. CSMB are antigen activated mature B cells that no longer express IgM or IgD and instead express the immunoglobulins IgG, IgA or IgE. They are significant antibody producers. Plasmablasts are also mature B cells which are significant antibody producers, being at a later stage of maturity than CSMBs. A reduction in levels of CSMB indicates that clozapine has an effect on the pathways involved in B cell maturation on the way to the production of mature plasma cells. B cells are also professional antigen presenting cells and cytokine producers and have a role in CD4 T cell priming. The inventors' new data also demonstrates an effect of the drug in reducing total IgG, IgA and IgM levels after administration. With the lack of effect on other B cells, shown by the lack of depletion of other sub-types and total B cell numbers but with a particular reduction in CSMBs and plasmablasts, this observation strongly supports a functional effect on CSMBs and plasmablasts which are central to long lived production of pathogenic antibodies in pathogenic immunoglobulin driven B cell disease with a T cell component.

### Impact on Class-Switched Memory B Cells and Antibody Production

Reduction in CSMBs by clozapine will consequently reduce the numbers of ASCs, and hence the secretion of specific immunoglobulins including the pathogenic immunoglobulins. Clozapine was also observed to cause a reduction in levels of plasmablasts, another type of mature B cell. This functional effect on persistent and long lived adaptive B cell and plasma cell function may ameliorate the diseases driven by the persistent generation of pathogenic immunoglobulins that drives the pathology of pathogenic immunoglobulin driven B cell diseases. The inventors' new data demonstrates a very significant effect on the number of circulating class switched memory B cells, a substantial effect on the number of plasmablasts and importantly, through the lack of recall response to common vaccines, an effect on the function of the class switched memory B cells and plasmablasts resulting in specific reduction of antibodies targeting a previously exposed antigen. The inventors' new data also demonstrates an effect of the drug in reducing total IgG, IgA and IgM levels after administration. With the lack of effect on other B cells, shown by the lack of depletion of other sub-types and total B cell numbers but with a particular reduction in CSMBs and plasmablasts, this observation strongly supports a functional effect on CSMBs and plasmablasts which are central to long lived production of pathogenic antibodies in pathogenic immunoglobulin (particularly IgG and IgA) driven B cell diseases.

The inventors' finding of a marked reduction in class-switched memory B cells in patients treated with clozapine indicates a robust impact on the process of immunoglobulin class switching. This has particular therapeutic relevance in pathogenic immunoglobulin driven B cell diseases in which class switch recombination (CSR) following the germinal centre reaction in secondary lymphoid organs provides antigen-primed/experienced autoreactive memory B cells and a core pathway for development and/or maintenance of autoimmunity. Further, this also has particular therapeutic relevance since the B lymphoid kinase haplotypes associated with B cell-driven autoimmune disorders exhibit an expansion of class-switched memory B cells and disease models of intrinsic B cell hyperactivity are associated with spontaneous CSR as associated with high titres of IgG autoantibodies effect of clozapine to both impact on CSR and lower IgG is of especial therapeutic potential in the setting of pathogenic immunoglobulin-driven B cell diseases where an impact on both the autoimmune memory repertoire and pathogenic immunoglobulin is desirable.

### Impact on IgA

The inventors have identified a significantly reduced circulating total IgA in patients treated with clozapine (leftward shift in immunoglobulin distribution) which notably demonstrated disproportionate lowering of IgA compared to that found with IgG and IgM. Substantiating the functional impact of this, the inventors have also identified a highly significant reduction in pneumococcal-specific IgA in patients treated with clozapine compared to clozapine-naive patients taking other antipsychotics. Recapitulating this in a model mammalian system, the inventors demonstrate that dosing of wild type mice with clozapine results in a significant reduction in circulating IgA compared to control or haloperidol treatment. While present at a relatively lower concentration in plasma compared to other immunoglobulin isotypes, IgA forms the great majority of all mammalian immunoglobulin, with ^{˜}3 g/day produced in human.

The inventors' finding of a significant reduction in total IgA in response to clozapine treatment reflects an important effect of clozapine on the function of IgA⁺ plasma cells. The generation of such cells occurs in both bone marrow and intestinal mucosae.

The inventors' identification of a significant impact of clozapine on plasma cell populations indicates the clear potential to modulate the diverse antibody-independent effector functions of B cells relevant to (auto)immune-mediated disease also.

### Impact on Plasmablast Antibody-Secreting Cells

The inventors have found that clozapine exerts a profound effect on reducing levels of circulating plasmablasts in patients. Accordingly, the inventors' observation of a profound impact of clozapine use on circulating plasmablast number highlights the potential for clozapine to modulate pathogenic immunoglobulin-driven B cell disease through both effects on circulating plasmablast secretion of immunoglobulin as well as interference with the potent function of plasmablasts to promote Tfh function.

### Impact on Long-Lived Plasma Cells

Using a wild type murine model, the inventors have found that regular clozapine administration in mice significantly reduces the proportion of long-lived plasma cells in bone marrow, an effect not seen with use of a comparator antipsychotic agent (haloperidol). Notably, human bone marrow resident long-lived PCs are long-regarded as the primary source of circulating IgG in human, thus providing a clear substrate for the inventors' observation of reduction in IgG in patients treated with clozapine. The inventors' observation of a specific effect of clozapine to deplete bone marrow long-lived plasma cells has, via an impact on long-lived plasma cell (autoreactive) memory, substantial therapeutic potential in pathogenic immunoglobulin driven B cell disease to eliminate inflammation and achieve remission.

### Impact on B Cell Precursors in Bone Marrow and Splenic Immature/Transitional Cells

The inventors identify a clear impact of clozapine on bone marrow B cell precursors after dosing of wild type mice. Specifically, an increase in the proportion of pre-pro B cells, in conjunction with a reduction in pre-B cells, proliferating pre-B cells and immature B cells in bone marrow. Together, these findings suggest a specific impact of clozapine on early B cell development, with a partial arrest between the pre-pro-B cell and pre-B cell stages in the absence of specific immunological challenge. The inventors have discerned an impact of clozapine to reduce the proportion of splenic T1 cells in wild type mice. Mirroring the murine findings, the inventors' interim findings from an ongoing observational study of patients on clozapine reveal a significant reduction in circulating transitional B cells. The human circulating transitional B cell subpopulation exhibits a phenotype most similar to murine T1 B cells and is expanded in patients with SLE.

Accordingly, the inventors' observation of an impact of clozapine to reduce the proportions of bone marrow B cell progenitors and immature (T1) splenic B cells provides additional anatomic compartmental origins beyond germinal centres for their finding of a reduction in circulating class-switched memory B cells and immunoglobulin in patients treated with clozapine. The therapeutic potential of this is further underlined by the consideration that the majority of antibodies expressed by early immature B cells are self-reactive.

### Lack of Direct B Cell Toxicity In Vitro

The inventors' new data using an in vitro B cell differentiation system to assess the specific impact of clozapine, its metabolite (N-desmethylclozapine) and a comparator antipsychotic control drug (haloperidol) further demonstrate: no direct toxicity effect of clozapine or its metabolite on differentiating B cells, no consistent effect on the ability of differentiated ASCs to secrete antibody and no consistent inhibitory effect on functional or phenotypic maturation of activated B cells to an early PC state in the context of an established in vitro assay.

Limited to the context of these in vitro experiments, these data suggest that clozapine is unlikely to be acting in a direct toxic manner on plasma cells or their precursors (e.g. via a cell intrinsic effect) to induce the effects observed on immunoglobulin levels. The observations suggest that clozapine's effect on B cells is more nuanced than existing B cell targeting therapies used for autoimmune disease which result in substantial depletion of multiple B cell subpopulations (e.g. rituximab and other anti-CD20 biosimilars) whose efficacy is mediated via direct effects on B cells such as signalling induced apoptosis, complement-mediated cytotoxicity or antibody-dependent cellular cytotoxicity.

Such a lack of apparent substantial direct toxicity by clozapine has a number of potential therapeutic advantages for clozapine, including reduced risk of generalised immunosuppression associated with indiscriminate B cell depletion (including elimination of protective B cells), and the potential to avoid maladaptive alterations observed with use of conventional B cell depleting therapies.

Efficacy in Collagen-Induced Arthritis (CIA) Mouse Model, Relevance of CIA as a Model of Pathogenic Immunoglobulin-Driven B Cell Disease with a T Cell Component and Importance of B Cell-T Cell Interactions in Autoimmunity

CIA is a well-established experimental model of autoimmune disease that results from immunisation of genetically susceptible strains of rodents and non-human primates with type II collagen (CII) (Brand et al., 2004)—a major protein component of cartilage—emulsified in complete Freund's adjuvant. This results in an autoimmune response accompanied by a severe polyarticular arthritis, typically 18-28 days post-immunisation and monophasic, resolving after ^{˜}60 days in mice (Bessis et al., 2017; Brand et al., 2007). The pathology of the CIA model resembles that of rheumatoid arthritis, including synovitis, synovial hyperplasia/pannus formation, cartilage degradation, bony erosions and joint ankylosis (Williams, 2012).

The immunopathogenesis of CIA is dependent on B cell-specific responses with generation of pathogenic autoantibodies to CII, in addition to involving T cell-specific responses to CII, FcγR (i.e. Fc receptors for IgG) and complement. The critical role of B cells in the development of CIA is substantiated by the complete prevention of development of CIA in mice deficient for B cells (IgM deleted), notwithstanding an intact anti-CII T cell response (Svensson et al., 1998). Moreover, the development of CIA has been shown to be absolutely dependent on germinal centre formation by B cells, with anti-CII immunoglobulin responses themselves largely dependent on normal germinal centre formation (Dandah et al., 2018; Endo et al., 2015). B cells have also been implicated in other aspects of CIA pathology, including bone erosion through inhibition of osteoblasts (Sun et al., 2018b). As a corollary, B cell depletion using anti-CD20 monoclonal antibodies prior to CII immunisation delays onset and severity of CIA, in conjunction with delayed autoantibody production (Yanaba et al., 2007). In this model, B cell recovery was sufficient to result in pathogenic immunoglobulin production after collagen-immunisation and associated development of disease.

The fundamental role played by collagen-specific IgG autoantibodies in the pathogenesis of CIA are highlighted by the observations that passive transfer of anti-CII serum or polyclonal IgG immunoglobulin to unimmunised animals results in arthritis (Stuart and Dixon, 1983), whilst lack of the FcγR chain near completely abrogates development of CIA in mice (Kleinau et al., 2000). In addition, introduction of pathogenic antibodies (i.e. collagen antibody-induced arthritis, CAIA) into germinal centre-deficient mice results in arthritis, demonstrating the ability of pathogenic antibody to largely circumvent the requirement for the germinal centre reaction (Dandah et al., 2018). Moreover, even mice lacking adaptive immunity (i.e. B and T cells), are susceptible to induction of CIA (Nandakumar et al., 2004).

Dynamic interactions between B cells and T cells are critical to an adaptive immune response and contribute to pathogenic immunoglobulin production in disease. Exemplifying this is the germinal cell reaction through which high affinity long-lived memory B cells and plasma cells are generated. B cell differentiation to these distal mature cell types requires both B cell activation and multi-stage selection/survival signals provided by mature T follicular helper cells to germinal centre B cells delivered focally via immunological synapses enabling kinetic, temporal and spatial segregation of multiple (bidirectional) signalling/co-stimulatory molecules and cytokines (Allen et al., 2007), including CD40L-CD40 (Foy et al., 1994), IL-21 (the most potent cytokine promoting plasma cell differentiation) (Ettinger et al., 2005; Kuchen et al., 2007; Zotos et al., 2010), PD-1/PD-L1 (Dorfman et al., 2006; Good-Jacobson et al., 2010), ICOS-ICOSL (Choi et al., 2011; Liu et al., 2015; Xu et al., 2013), SLAM (signaling lymphocyte activation molecule) family receptors (Cannons et al., 2010) required for sustained B cell:T cell adhesion and others. This process of ‘entanglement’ is critical to selective delivery of helper signals to high affinity, non-autoreactive B cell clones to select for plasma cell differentiation. Underlining the importance of T follicular helper cells (T_{FH}) in the generation of B cell memory, T_{FH} cells and their PI3Kδ activity are the primary limiting factor in germinal centre development (Rolf et al., 2010). T_{FH} cells also secrete class switch factors required to instruct class switch recombination of B cells (Crotty, 2011), including IL-4 for IgG1 (Reinhardt et al., 2009) and IgE, IL-21 for IgG3, IgA and IgE (Avery et al., 2008; Pene et al., 2004). Notably, the process of B cell-T cell interaction in lymphoid tissue is not restricted to germinal centre T_{FH}-germinal centre B cell interactions, but also includes (Tangye et al., 2015): extrafollicular T cell help to plasmablasts via IL-21 and Bcl-6 (Lee et al., 2011) supported by stromal cell-derived APRIL (Zhang et al., 2018), T_{FH}-non-cognate B cell interactions in the follicular mantle and cognate interactions at the T-B border. Notably these interactions are not solely unidirectional; thus, circulating plasmablasts can reciprocally modulate T_{FH} cells and promote the T_{FH} differentiation programme via secretion of IL-6 (Chavele et al., 2015). This positive feedback loop and the earlier observations underline the interdependence of B cell and T cell responses to physiological and pathological immunoglobulin production and the genesis/perpetuation of autoimmunity.

Cognate interactions between B cells and T cells are recognised as critical to the induction of CIA. Accordingly, blocking the interaction of CD40 ligand (gp39) expressed on the surface of CD4⁺ T (helper) cells with CD40 on the surface B cells using monoclonal anti-CD40-L antibodies is sufficient to completely prevent the development of CIA in mice with associated reduction in pathogenic anti-CII antibodies (Durie et al., 1993). Similarly, T cell-B cell ICOS signalling has been shown to be necessary for the induction and maintenance of CIA in mice (Panneton et al., 2018); as a corollary, inhibition of the ICOS/ICOS-L interaction reduces disease severity and progression in mice (O'Dwyer et al., 2018). Further, IL-21 knockout mice are resistant to the development of CIA and exhibit lower IgG anti-CII antibodies, with 11-21 signalling in B cells shown to be responsible for CIA development (Sakuraba et al., 2016).

An additional T cell population shown to play a role in (suppression of) humoral immunity are Foxp3⁺ regulatory T cells (Tregs). Underlining the importance of Tregs, their depletion using anti-CD25 or diphtheria toxin results in potent induction of autoantibodies, enhanced T_{FH} cell and germinal centre responses and histological evidence of autoimmunity (Leonardo et al., 2012; Sakaguchi et al., 1995). Specifically, within secondary lymphoid tissue T follicular regulatory cells residing at the T cell zone-B cell follicle border and B cell follicle (Sayin et al., 2018) act to inhibit antibody production through multiple interactions with B cells and T_{FH} cells, with mechanisms proposed (Wing et al., 2018) including: direct suppression of follicular b cells, prevention of T_{FH} cell germinal centre entry and inhibition of B cell differentiation in the germinal centre itself. Regulatory T cells therefore modulate the differentiation of antibody secreting cells via germinal centres through their co-option of the T_{FH} differentiation pathway (Chung et al., 2011; Linterman et al., 2011). Underlining the importance of Treg cells in the pathogenesis of CIA, adoptive transfer of antigen-specific Treg cells inhibits the progression of CIA (Sun et al., 2018a).

The present inventors have found that clozapine leads to a significant reduction in the proportion of B cells in lymph nodes of mice immunised with heterologous type II collagen. Concordant findings of smaller magnitude were evident in spleen. A similar reduction was observed when dosing healthy wild type mice with clozapine without predilection for a particular major B cell subset, suggesting an influence of clozapine to reduce major secondary lymphoid tissue B cell subsets.

The inventors' data also shows a highly significant ability of clozapine to reduce the proportion of germinal centre B cells, together with a very significant dose-dependent reduction in their levels of activation, as judged by their expression of the GL7 activation antigen/epitope. Notably GL7^{hi} B cells show greater specific and total antibody production in addition to greater antigen presenting capacity. Accordingly, the inventors' finding suggests that clozapine has effects on both the abundance of germinal centre B cells as well as their functionality, with both effects converging to inhibit effective germinal centre function and/or formation.

In addition, the inventors have identified an additional effect of clozapine on the other major cell type critical for germinal centre formation and function, namely T follicular helper cells (T_{FH}). They find that clozapine substantially reduces expression of key T_{FH} markers, PD-1 (programmed cell death-1) and CXCR5 without an perturbation in the proportion of T_{FH} cells in secondary lymphoid tissue. T_{FH} cells express PD-1 at high levels (and upregulate expression soon after antigen stimulation) where it serves to critically regulate T_{FH} position and function in the germinal centre. Specifically, when engaged by surrounding follicular B cells which constitutively express the PD-1 ligand (PD-L1), PD-1 acts to inhibit T cell recruitment into the follicle thereby concentrating T_{FH} cells into the germinal centre itself. This is critical for T_{FH} cells to undertake their proper role to support germinal centre B cells. PD-1 is also required for optimal IL-21 production by T_{FH} cells. As a corollary PD-1 deficient mice have fewer long-lived plasma cells, in part due to greater germinal centre cell death. Within the germinal centre the PD-1/PD-L1 interaction also serves to optimise B cell competition and affinity maturation.

Concordantly, the inventors also observe a highly significant impact of clozapine to reduce expression of CXCR5 on T_{FH} cells. CXCR5 is regarded as a defining marker for T_{FH} cells and is required for T cell follicular homing. Notably T cells deficient in CXCR5, while able to access the follicular germinal centre, are inefficient at supporting GC responses.

Thus, the inventors' findings indicate that clozapine exerts an inhibitory influence on T_{FH} functionality and germinal centre formation, at least in part through altered expression of PD-1 and CXCR5. The findings indicate that clozapine reduces the ability of T_{FH} cells to concentrate within the germinal centre to provide B cell help to support differentiation of antigen specific B cells into plasma cells and memory cells and lowers the efficiency thereof, thereby exerting a potent inhibitory influence on antibody dependent immune responses.

In addition, the inventors show that clozapine increases the proportion of Foxp3⁺ regulatory T cells, an immune suppressive T cell population, (Tregs) in secondary lymphoid tissue (draining lymph node and spleen) in addition to upregulating expression of CD25 on Foxp3⁺ Tregs. In the context of lymphoid follicles, Foxp3⁺ T follicular regulatory cells (Tfr) regulate the germinal centre reaction, serving to limit germinal centre B cell and T_{FH} numbers, and inhibit antibody affinity maturation, plasma cell differentiation and antigen-specific immunoglobulin secretion. Accordingly, the inventors' findings suggest that clozapine is likely to act in part through Treg-B cell interaction (in addition to provision of T cell help to B cells) to dampen humoral immune responses.

Accordingly, the inventors have employed the CIA model as a highly clinically relevant experimental system in which B cell-derived pathogenic immunoglobulin made in response to a sample antigen following B cell-T cell interaction (including in draining lymph node germinal centres) (Dandah et al., 2018) drives autoimmune pathology to explore the potential efficacy of clozapine and its associated cellular mechanisms. The inventors demonstrate that clozapine delays the onset and reduces the incidence of CIA in mice, an effect most apparent when dosed just after CII immunisation. Furthermore, the inventors' data indicates that clozapine reduces the severity of CIA, judged by number of affected paws and clinical severity score. The inventors identify important effects of clozapine on key cell types implicated in the pathogenesis of CIA, including a reduction in the proportion of splenic plasma cells and highly significant reduction in germinal centre B cells in local draining lymph node. Moreover, the inventors' findings demonstrate reduced markers of functional activity for antibody production and antigen presentation on lymph node germinal centre B cells in response to clozapine in CII immunised mice. Measured at a single time point, they also observe a significant reduction in anti-collagen IgG1 antibody levels. Together, the inventors' findings in the CIA model point to a specific ability of clozapine to favourably impact upon pathogenic immunoglobulin B cell-driven pathology and thereby B cell mediated disorders in which autoantibody formation is a key component.

Thus, the present invention provides a compound selected from clozapine, norclozapine and prodrugs thereof and pharmaceutically acceptable salts and solvates thereof for use in the treatment or prevention of a pathogenic immunoglobulin driven B cell disease with a T cell component in a subject, in particular, wherein said compound causes mature B cells to be inhibited in said subject.

### BRIEF DESCRIPTION OF THE DRAWINGS

FIG. 1A-C. show the relative frequencies of numbers of patients at each serum concentration value for IgG, IgA and IgM respectively for clozapine-treated patients (black) and clozapine-naïve patients (grey) (see Example 1).

FIG. 1D. illustrates density plots showing the distribution of serum immunoglobulin levels in patients receiving clozapine referred for Immunology assessment (light grey left-most curve, n=13) following removal of 4 patients (n=2 with haematological malignancy and n=2 previously included within the inventor's recent case-control study (Ponsford et al., 2018a). Serum immunoglobulin distributions for clozapine-treated (mid-grey middle curve, n=94) and clozapine-naive (dark grey right-most curve, n=98) are also shown for comparison [adapted from (Ponsford et al., 2018a)]. Dotted lines represent the 5th and 95th percentiles for healthy adults (see Example 1).

FIG. 2. shows the effect of duration of clozapine use on serum IgG levels (see Example 1).

FIG. 3A. shows the number of class switched memory B cells (CSMB) (CD27+/IgM−/IgD−, expressed as a percentage of total CD19+ cells) in healthy controls, in patients taking clozapine referred to clinic and in patients with common variable immunodeficiency disorder (CVID) (see Example 1).

FIG. 3B. shows B cell subsets, expressed as a percentage of total CD19⁺ cells, in patients with schizophrenia with a history of clozapine therapy referred to clinic (numbers as shown), common variable immunodeficiency (CVID, n=26) and healthy controls (n=17). B-cell subsets gated on CD19⁺ cells and defined as follows: Naïve B-cells (CD27⁻IgD⁺IgM⁺), Marginal Zone-like B-cells (CD27⁺IgD⁺IgM⁺), Class-switched Memory B-cells (CD27+IgD⁻IgM⁻), and Plasmablasts (CD19⁺CD27^{Hi}IgD⁻). Non-parametric Mann-Whitney testing performed for non-normally distributed data, * p<0.05, ** p<0.01, *** p<0.001, ****p<0.0001 (see Example 1).

FIG. 4A. shows the number of plasmablasts (CD38+++/IgM-, expressed as a percentage of total CD19+ cells) in healthy controls, in patients taking clozapine referred to clinic and in patients with common variable immunodeficiency disorder (CVID) (see Example 1).

FIG. 4B. illustrates vaccine specific-IgG response assessment (see Example 1).

FIG. 5. shows gradual recovery of serum IgG post-discontinuation of clozapine from 3.5 to 5.95 g/L over three years. LLN=lower limit of normal (see Example 1).

FIG. 6A-C. shows interim data findings on the levels of circulating IgG, IgA and IgM in patients on non-clozapine antipsychotics (‘control’, left) versus clozapine (right). Mean±SEM (see Example 2).

FIG. 7. shows interim data findings on peripheral blood levels of pneumococcal-specific IgG in patients on non-clozapine antipsychotics (‘control’, left) versus clozapine (right). Mean±SEM (see Example 2).

FIG. 8A-B. shows interim data findings on peripheral blood levels of B cells (CD19⁺) in patients on non-clozapine antipsychotics (‘control’, left) versus clozapine (right), expressed as absolute levels and as a percentage of lymphocytes (%, i.e. of T+B+NK cells). Mean±SEM (see Example 2).

FIG. 9A-C. shows interim data findings on peripheral blood levels of naive B cells (CD19⁺/CD27⁻) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 10A-C. shows interim data findings on peripheral blood levels of memory B cells (CD19⁺/CD27⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 11A-C. shows interim data findings on peripheral blood levels of class switched (CS) memory B cells (CD27⁺/IgM⁻/IgD⁻) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 12A-C. shows interim data findings on peripheral blood levels of IgM high IgD low (CD27⁺/IgM⁻/IgD⁻) memory B cells, i.e. post-germinal centre IgM only B cells, in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 13A-C. shows interim data findings on peripheral blood levels of transitional B cells (IgM⁺⁺/CD38⁺⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 14A-C. shows interim data findings on peripheral blood levels of marginal zone (MZ) B cells (CD27⁺/IgD⁺/IgM⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 15A-C. shows interim data findings on peripheral blood levels of plasmablasts in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 16. shows the body weight growth curve of WT mice in response to clozapine at different doses versus haloperidol and vehicle controls. Mean±SEM (see Example 3).

FIG. 17. shows body weight comparisons of WT mice at days 3, 12 and 21 of treatment. Mean±SEM (see Example 3).

FIG. 18. shows the impact of clozapine versus haloperidol and vehicle control on overall B cell content and pre-pro B cell and pro B cell precursors in bone marrow of WT mice. Mean±SEM (see Example 3).

FIG. 19. shows the impact of clozapine versus haloperidol and vehicle control on pre-B cells, proliferating B cells and immature B cell precursors in bone marrow of WT mice. Mean±SEM (see Example 3).

FIG. 20. shows the impact of clozapine versus haloperidol and vehicle control on class-switched memory B cells, plasmablasts and long-lived plasma cells in bone marrow of WT mice. Mean±SEM (see Example 3).

FIG. 21. shows the impact of clozapine versus haloperidol and vehicle control on overall B cells, T cells, other cell populations (TCR-β⁻/B220⁻) and activated T cells in spleen of WT mice. Mean±SEM (see Example 3).

FIG. 22. shows the impact of clozapine versus haloperidol and vehicle control on transitional (T1 and T2), follicular, marginal zone (MZ) and germinal centre (GC) B cells in spleen of WT mice. Mean±SEM (see Example 3).

FIG. 23. shows the impact of clozapine versus haloperidol and vehicle control on B cell subpopulations and T cells in the mesenteric lymph nodes (MLN) of WT mice. Mean±SEM. T1 and T2, transitional type 1 and type 2 B cells, respectively. MZ, marginal zone. GC, germinal centre (see Example 3).

FIG. 24. shows the impact of clozapine versus haloperidol and vehicle control on circulating immunoglobulins in WT mice. Mean±SEM (see Example 3).

FIG. 25. shows impact of clozapine on day of clinical onset of CIA. Mean±SEM (see Example 4).

FIG. 26. shows impact of clozapine on incidence of CIA (see Example 4).

FIG. 27. shows the impact of clozapine on the severity of CIA, judged by clinical score and thickness of first affected paw, in mice dosed from day 1 post-immunisation. Mean±SEM (see Example 4).

FIG. 28. shows the impact of clozapine on the severity of CIA, judged by number of affected paws by day of treatment with clozapine (day 15, D15 or day 1, D1) post-immunisation. Mean±SEM (see Example 4).

FIG. 29. shows the impact of clozapine versus control on B220⁺ (i.e. CD45⁺) cells in spleen and local lymph node of CIA mice. Mean±SEM (see Example 4).

FIG. 30. shows the impact of clozapine versus control on plasma cells (PC) in spleen and local lymph node of CIA mice. Mean±SEM (see Example 4).

FIG. 31. shows the impact of clozapine versus control on germinal centre (GC) B cells (B220⁺/IgD⁻/Fas⁺/GL7⁺) in spleen and local lymph node of CIA mice. Mean±SEM (see Example 4).

FIG. 32. shows the impact of clozapine versus control on expression of GL7 on germinal centre (GC) B cells (B220⁺/IgD⁻/Fas⁺/GL7⁺) in spleen and local lymph node of CIA mice. MFI, mean fluorescent intensity. Mean±SEM (see Example 4).

FIG. 33. shows the impact of clozapine versus control on peripheral blood anti-collagen IgG1 and IgG2a antibody levels of CIA mice (see Example 4).

FIG. 34. shows the impact of clozapine versus control on germinal centre resident T follicular helper cells (CD4⁺PD1⁺) in spleen and local lymph node of CIA mice. Mean±SEM (see Example 4).

FIG. 35. shows the impact of clozapine versus control on expression of PD1 on germinal centre resident T follicular helper cells (CD4⁺PD1⁺) in spleen and local lymph node of CIA mice. MFI, mean fluorescent intensity. Mean±SEM (see Example 4).

FIG. 36. shows the impact of clozapine versus control on expression of CXCR5 on germinal centre resident T follicular helper cells (CD4⁺PD1⁺) in spleen and local lymph node of CIA mice. MFI, mean fluorescent intensity. Mean±SEM (see Example 4).

FIG. 37. shows the impact of clozapine versus control on expression of CCR7 on germinal centre resident T follicular helper cells (CD4⁺PD1⁺) in spleen and local lymph node of CIA mice. MFI, mean fluorescent intensity. Mean±SEM (see Example 4).

FIG. 38. shows the impact of clozapine versus control on Treg (CD4⁺/CD25⁺/FoxP3⁺) cells in spleen and local lymph node of CIA mice. Mean±SEM (see Example 4).

FIG. 39. shows the impact of clozapine versus control on expression of CD25 on Tregs in spleen and local lymph node of CIA mice. MFI, mean fluorescent intensity. Mean±SEM (see Example 4).

FIG. 40. shows the impact of clozapine versus control on expression of FoxP3 on Tregs in spleen and local lymph node of CIA mice. MFI, mean fluorescent intensity. Mean±SEM (see Example 4).

FIG. 41. shows protocol schematic for in vitro generation/differentiation of human plasma cells (see Example 5).

FIG. 42. shows a schematic of the trial illustrating clozapine uptitration period followed by administration of typhoid vaccine (Typhim Vi) by injection (arrow) and then ongoing dosing with clozapine. Control cohort (vaccine only, no clozapine) and optional cohort (dose to be selected guided by findings from dose 1 and dose 3) (see Example 6).

### DETAILED DESCRIPTION OF THE INVENTION

The present invention also provides a method of treatment or prevention of a pathogenic immunoglobulin driven B cell disease with a T cell component in a subject by administering to said subject an effective amount of a compound selected from clozapine, norclozapine and prodrugs thereof and pharmaceutically acceptable salts and solvates thereof, in particular, wherein said compound causes mature B cells to be inhibited in said subject.

The present invention also provides use of a compound selected from clozapine, norclozapine and prodrugs thereof and pharmaceutically acceptable salts and solvates thereof in the manufacture of a medicament for the treatment or prevention of a pathogenic immunoglobulin driven B cell disease with a T cell component in a subject, in particular, wherein said compound causes mature B cells to be inhibited in said subject.

Clozapine or norclozapine may optionally be utilised in the form of a pharmaceutically acceptable salt and/or solvate and/or prodrug. In one embodiment of the invention clozapine or norclozapine is utilised in the form of a pharmaceutically acceptable salt. In a further embodiment of the invention clozapine or norclozapine is utilised in the form of a pharmaceutically acceptable solvate. In a further embodiment of the invention clozapine or norclozapine is not in the form of a salt or solvate. In a further embodiment of the invention clozapine or norclozapine is utilised in the form of a prodrug. In a further embodiment of the invention clozapine or norclozapine is not utilised in the form of a prodrug.

The term “pathogenic immunoglobulin B cell disease with a T cell component” includes B cell mediated disease, especially autoimmune disease, which involves pathogenic immunoglobulin (e.g. IgG, IgA and/or IgM) targeting a self-antigen (e.g. auto-antibody IgG, IgA and/or IgM) and with T cell mediated inflammation as a principal mechanism. The term also includes immune rejection of an allograft as in graft versus host disease.

The range of self-antigens involved in autoimmune diseases include myelin (multiple sclerosis), pancreatic beta cell proteins (Type 1 diabetes mellitus), fibrillarin (scleroderma), cardiolipin (systemic lupus erythematosus) and 2-hydrolase (autoimmune Addison's disease).

Exemplary pathogenic immunoglobulin driven B cell diseases with a T cell component may be the skin related diseases vitiligo, psoriasis, coeliac disease, dermatitis herpetiformis or discoid lupus erythematosus. Alternatively, the disease may be the muscle related diseases dermatomyositis or polymyositis. Alternatively, the disease may be the pancreas related disease Type 1 diabetes mellitus. Alternatively, the disease may be the adrenal gland related disease autoimmune Addison's disease. Alternatively, the disease may be the neurological related disease multiple sclerosis. Alternatively, the disease may be the lung related disease interstitial lung disease. Alternatively, the disease may be the bowel related diseases Crohn's disease or ulcerative colitis. Alternatively, the disease may be the thyroid related disease thyroid autoimmune disease. Alternatively, the disease may be the eye related disease autoimmune uveitis. Alternatively, the disease may be the liver related diseases primary biliary cirrhosis or primary sclerosing cholangitis. Alternatively, the disease may be undifferentiated connective tissue disease. Alternatively, the disease may be an immune-mediated inflammatory disease (IMID) such as scleroderma, rheumatoid arthritis or Sjogren's disease. Alternatively, the disease may be autoimmune thrombocytopenic purpura. Alternatively, the disease may be a connective tissue disease such as systemic lupus erythematosus. Alternatively, the disease may be mixed connective tissue disease (MCTD).

Alternatively, the disease may be graft versus host disease.

References highlighting the role of pathogenic immunoglobulins, B and T cells in the aforementioned diseases include:

### Vitiligo

Vitiligo is an acquired chronic depigmenting disease resulting from selective melanocyte destruction (Ezzedine et al., 2015).

Patients with vitiligo frequently exhibit autoantibodies at levels higher than controls, including anti-thyroperoxidase, anti-thyroglobulin, antinuclear, anti-gastric parietal cell and anti-adrenal antibodies (Liu and Huang, 2018), some of which correlate with clinical vitiligo activity (Colucci et al., 2014). In comparison to controls, vitiligo is associated with elevated total IgG, IgG1 and IgG2 and melanocyte-reactive antibodies (Li et al., 2016b). The latter are most frequently directed against pigment cell antigens (Cui et al., 1992), including melanin-concentrating hormone receptor 1 (Kemp et al., 2002). Melanocyte death in vitiligo has been proposed to reflect apoptosis and is promoted in vitro by serum IgG from vitiligo patients (Ruiz-Arguelles et al., 2007). Notably IgG (and C3) deposits have been observed in the basement membrane zone of lesional skin. Furthermore, binding of IgG from vitiligo patients to cultured melanocytes increases with disease extent and activity, with further correlation of vitiligo activity to levels of anti-melanocyte IgA (Kemp et al., 2007).

While there is debate regarded whether the presence of autoantibodies in vitligo reflects a primary cause or consequence of the disease, it is clear that vitiligo autoantibodies possess the capacity to result in pigment cell injury via multiple effector mechanisms, including antibody-dependent cellular cytotoxicity and complement-mediated cell damage in vitro (Cui et al., 1993; Norris et al., 1988).

MCHR function-blocking autoantibodies have also been identified in vitiligo patients, which would be expected to interfere with normal melanocyte function (Gottumukkala et al., 2006). In addition to the role of MCHR1 as a B cell autoantigen, the importance of B cells is further suggested in vitiligo through identification of Bcl-2 positive infiltrates in close juxtaposition to areas of depigmentation (Ruiz-Arguelles et al., 2007). Vitiligo has also been reported to respond to B cell depletion with monoclonal antibody to CD20 (Ruiz-Arguelles et al., 2013).

Notably T regulatory cells (Tregs) are deficient in vitiligo together with an increase in PD-1 expressing Tregs suggesting Treg exhaustion and a possible role in the pathogenesis of vitiligo (Tembhre et al., 2015). This loss of suppression correlates with hyperactivation of CD8⁺ cytolytic T cells which are known to play a key role in vitiligo-induced depigmentation (Lili et al., 2012).

### Primary Biliary Cirrhosis (PBC)

Primary biliary cirrhosis (PBC), also known as primary biliary cholangitis, is a chronic cholestatic liver disorder characterised pathologically by progressive small intrahepatic bile duct destruction with associated portal inflammation, fibrosis and risk of progression to cirrhosis, and serologically (>95%) by anti-mitochondrial antibody (AMA) and often an elevated serum IgM (Carey et al., 2015). Notably, autoantibodies (e.g. anti-centromere) are strongly associated with risk of progression to cirrhosis and portal hypertension (Nakamura, 2014).

While T cells have been reported to constitute the majority of cellular infiltrate in early PBC, B cells/plasma cells are also identified (Tsuneyama et al., 2017). Specifically, formation of follicle-like aggregations of plasma cells expressing IgG and IgM around intrahepatic ducts have been noted in patients with PBC, further correlating with higher titres of AMA (Takahashi et al., 2012). The finding of oligoclonal B cell proliferation and accumulation of somatic mutations in liver portal areas from patients with PBC is consistent with antigen-driven B cell responses (Sugimura et al., 2003). A sustained rigorous B cell response in PBC has also been suggested through the finding of high levels of autoantigen-specific peripheral plasmablasts (to the pyruvate dehydrogenase complex autoantigen PDC-E2) consistent with ongoing activation of autoreactive B cells (Zhang et al., 2014).

Notably, newly diagnosed patients with PBC exhibit elevated numbers of circulating T follicular helper cells and plasma cells, with both correlating positively with each other, as well as with levels of serum AMA and IgM (Wang et al., 2015). Rituximab has been reported to reduce serum total IgG, IgA and IgM, in addition to AMA IgA and IgM in patients with PBC and an incomplete response to ursodeoxycholic acid (Tsuda et al., 2012), in addition to a limited but discernible favourable effect on alkaline phosphatase and pruritus (Myers et al., 2013).

### Primary Sclerosing Cholangitis (PSC)

PSC is a chronic liver disorder characterised by multifocal biliary strictures and high risk of cholangiocarcinoma, together with strong association with inflammatory bowel disease (Karlsen et al., 2017). A large number of autoantibodies have been detected in patients with PSC, but generally of low specificity, including pANCA, ANA, SMA and anti-biliary epithelial cell (Hov et al., 2008). Notably and consistent with the known physiologically dominant role for secreted IgA in bile, the presence of autoreactive IgA against biliary epithelial cells correlates with faster clinical progression of PSC (to death/liver transplantation) (Berglin et al., 2013).

Functional IgA, IgM and IgG antibody secreting cells have been identified in PSC liver explants (Chung et al., 2016). Notably, the majority of these cells are plasmablasts rather than plasma cells (Chung et al., 2017). Alterations in the peripheral circulating T follicular helper cell compartment, a key facilitator of antibody responses, have been identified in PSC (Adam et al., 2018). Supporting a role for shared liver and gut adaptive immune response in PSC associated with inflammatory bowel disease, B cells of common clonal origin have been identified in both tissues together with evidence of higher somatic hypermutation consistent with (same) antigen-driven activation (Chung et al., 2018).

As with PBC, a contribution from T follicular helper (T_{FH}) cells to disease pathogenesis is suggested by the presence of potentially pathogenic TFH cells (CCR7^{lo}CXCR5⁺PD-1⁺CD4⁺ T cells) (Adam et al., 2018). Notably genetic and functional data also support a role for impaired Foxp3⁺ regulatory T cell (Treg) function in contributing to the immune dysregulation of PSC (Sebode et al., 2014).

Notably PSC is also considered part of the spectrum of IgG4-related diseases (Gidwaney et al., 2017), a multiorgan fibroinflammatory disorder which is also associated with autoimmune pancreatitis and a robust elevation in circulating plasmablasts/plasma cells. Which reduce following treatment with glucocorticoids (Lin et al., 2017). This is associated with both an increase in class-switched memory B cells and T_{FH} cells, with IgG levels correlating to both circulating plasmablast and T_{FH} frequency and evidence of a marked tissue T_{FH} cell infiltration (Kubo et al., 2018). Substantiating the role of B cells in IgG4-related disease, B cell depletion with rituximab is effective in both induction and treatment of relapses (Ebbo et al., 2017).

### Autoimmune Thrombocytopenic Purpura (Immune Thrombocytopenia; Adult Immune Thrombocytopenia)

Immune thrombocytopenia (ITP) is a disorder characterised by acquired thrombocytopenia (low platelet count) driven by immune recognition of platelet autoantigens and ensuing destruction of platelets.

Highlighting the importance of humoral immune mechanisms were early studies revealing that infusion of serum from patients with ITP to healthy volunteers resulted in profound thrombocytopenia, that this was dose-dependent, that the humoral factor could be adsorbed by platelets and in the IgG fraction (Harrington et al., 1951; Karpatkin and Siskind, 1969; Shulman et al., 1965). In addition to IgG autoantibodies against platelet glycoprotein (GP) IIb/IIIa, IgA and IgM anti-platelet autoantibodies have been identified (He et al., 1994), as well as against other platelet surface proteins such as GPIb/IX, with a high degree of specificity for ITP (McMillan et al., 2003). These autoantibodies result in antibody-dependent platelet phagocytosis seen in vitro (Tsubakio et al., 1983) and in vivo by splenic macrophages and peripheral neutrophils (Firkin et al., 1969; Handin and Stossel, 1974). Notably the amount of platelet-associated IgG inversely correlates with the platelet count (Tsubakio et al., 1983).

In addition to promoting platelet destruction, autoantibodies have also been demonstrated to directly affect bone marrow megakaryocyte maturation (Nugent et al., 2009). Both GPIIb/IIIa and GPIb/IX are expressed on megakaryocytes, with autoantibodies found binding to these in ITP (McMillan et al., 1978). Furthermore, plasma from patients with ITP suppresses megakaryocyte production and maturation in vitro, an effect ameliorated through adsorption of autoantibody with immobilised antigen and also seen with patient IgG but not control IgG (McMillan et al., 2004).

Splenectomy samples from patients with ITP show marked follicular hyperplasia with germinal centre formation and increased plasma cells consistent with an ongoing active B cell response in ITP (Audia et al., 2011). Notably, frequency of splenic T follicular helper cells is higher in ITP compared to controls, with further expansions in splenic pre-germinal centre B cell, germinal centre B cell (in addition to plasma cells) also identified, and all correlating positively with percentage of T follicular helper cells (Audia et al., 2014). B cell depletion with rituximab is effective in improving platelet count in ^{˜}60% of patients with ITP, with patients in whom autoantibody is persistent more frequently failing to demonstrate a clinical response (Arnold et al., 2017; Khellaf et al., 2014).

Highlighting an important role for long-lived plasma cells as a substrate for ongoing generation of pathogenic autoantibodies mediating platelet destruction and reduced production, patients who are refractory to B cell depletion with rituximab display autoreactive anti-GpIIb/IIIa plasma cells in spleen expressing a long-lived genetic programme (Mahevas et al., 2013).

T cells make an important contribution to the pathogenesis of ITP, with evidence of prolonged survival of autoreactive T cells and deficient Treg function (Wei and Hou, 2016).

### Autoimmune Addison's Disease (AAD)

AAD is a rare autoimmune endocrinopathy characterised by an aberrant immune destructive response against adrenal cortical steroid producing cells (Mitchell and Pearce, 2012).

A major autoantigen in AAD is steroid 21-hydroxylase with the majority (>80%) of patients exhibiting autoantibodies against this (Dalin et al., 2017), with sera from patients with AAD reacting with the zona glomerulosa of the adrenal cortex (Winqvist et al., 1992). Anti-adrenal antibodies are predictive of progression to overt disease or subclinical adrenal insufficiency in patients with other autoimmune disorders (Betterle et al., 1997). Notably, levels of adrenal autoantibodies correlate with severity of adrenal dysfunction, suggesting association with the destructive phase of autoimmune adrenalitis. Conversely, patients exhibiting biochemical remission of adrenal dysfunction, including in response to corticosteroid therapy, also display loss of adrenal cortex autoantibody and 21-hydroxylase autoantibody (De Bellis et al., 2001; Laureti et al., 1998). While it is unclear whether these autoantibodies are directly pathogenic (particularly given their intracellular target), organ-specific reactive antibodies have been demonstrated from AAD sera (Khoury et al., 1981).

Histologically, AAD is characterised by a diffuse inflammatory infiltrate, including plasma cells (Bratland and Husebye, 2011).

Genetic support for an important role for B cells in the susceptibility to AAD has come from the identification of BACH2 as a major risk locus (Eriksson et al., 2016; Pazderska et al., 2016). BACH2 encodes a transcriptional repressor which is required for class switch recombination and somatic hypermutation in B cells through regulation of the B cell gene regulatory network (Muto et al., 2010; Muto et al., 2004). Administration of rituximab to induce B cell depletion in AAD has reported efficacy in a new-onset case, with evidence of sustained improvement in cortisol and aldosterone (Pearce et al., 2012).

Supporting a T cell component to the pathogenesis of AAD, a high frequency of 21-hydroxylase-specific T cells is identifiable in patients, with CD8⁺ T cells able to lyse 21-hydroxylase positive target cells (Dawoodji et al., 2014).

### Multiple Sclerosis (MS)

MS is an inflammatory demyelinating disorder of the central nervous system (CNS).

While MS is typically conceptualised as a CD4 Th1/Th17 T cell-mediated disorder, largely based on findings using the experimental autoimmune encephalomyelitis (EAE) model, T cell-specific therapies have not demonstrated clear efficacy in relapsing-remitting MS (Baker et al., 2017). In contrast, many active MS immunomodulatory and disease-modifying therapies are recognised to affect the B cell compartment and/or serve to deplete memory B cells, either physically or functionally (Baker et al., 2017; Longbrake and Cross, 2016).

The most well-recognised and persistent immunodiagnostic abnormality in MS—the presence of oligoclonal bands in cerebrospinal fluid (CSF) typically of IgG isotype (but also IgM)—is a product of B lineage cells (Krumbholz et al., 2012). Notably clonal IgG in CSF is stable over time, consistent with local production from resident long-lived plasma cells or antibody secreting cells maturing from memory B cells (Eggers et al., 2017). That anti-CD20 therapy reduces CSF B cells with no significant impact on oligoclonal bands suggests a substantial role for long-lived plasma cells in oligoclonal band production (Cross et al., 2006). Correlation of immunoglobulin proteomes in CSF samples has revealed strong overlap with transcriptome of CSF B cells highlighting the latter as the source (Obermeier et al., 2008). The majority of B cells in the CSF of patients with MS are memory B cells and short-lived plasmablasts, with the latter representing the main source for intrathecal IgG synthesis and correlating with parenchymal inflammation revealed by MRI (Cepok et al., 2005), with evidence of greater involvement in acute inflammation associated with relapsing-remitting MS (Kuenz et al., 2008).

Pathologically, organised ectopic tertiary lymph node-like structures with germinal centres are present in the cerebral meninges in MS (Serafini et al., 2004). As with parenchymal lesions, B cell clones in meningeal aggregates largely use IgG (^{˜}90%, remainder IgM) (Lovato et al., 2011). Moreover, antigen experienced B cell clones are shared between these meningeal aggregates and corresponding parenchymal lesions (Lovato et al., 2011). In addition, flow cytometry with deep immune repertoire sequencing of peripheral blood and CSF B cells indicate that peripheral class-switched B cells, including memory B cells, have a connection to the CNS compartment (Palanichamy et al., 2014). Notably memory B cells have recently been demonstrated to promote autoproliferation of Th1 brain-homing autoreactive CD4⁺ T cells in MS (Jelcic et al., 2018).

The best characterised autoantigen in MS is myelin oligodendrocyte glycoprotein (MOG), the target of autoantibodies in EAE and against which antibodies are identified in ^{˜}20% children but relatively few adults with demyelinating disorders (Krumbholz et al., 2012; Mayer and Meinl, 2012). Evidence supporting a role for pathogenic autoantibody in MS includes the efficacy of plasma exchange in some patients (Keegan et al., 2005) and the presence of complement-dependent demyelinating/axopathic autoantibodies in a subset of patients with MS (Elliott et al., 2012). Other autoantibodies have been identified against axoglial proteins around the node of Ranvier including autoantibodies against contactin-2 and neurofascin, with evidence of axonal injury evident using in vivo models when transferred with MOG-specific encephalitogenic T cells and inhibition of axonal conduction when used with hippocampal slices in vitro (Mathey et al., 2007).

Substantiating a key role for B cells in relapsing-remitting MS, B cell depletion using the chimeric anti-CD20 antibody rituximab reduces both inflammatory brain lesions and clinical relapses (Hauser et al., 2008). Similar unequivocally positive efficacy findings have been observed with use of other CD20 depleting agents such as ocrelizumab (humanised monoclonal anti-CD20 antibody) in relapsing MS (Hauser et al., 2017) and primary progressive MS (Montalban et al., 2017).

Illustrating cross-talk between B cells and T cells in MS, circulating TFH cells are expanded in MS, correlating with progression of disease, and also present in lesions where they can promote inflammatory B cell function including antibody secretion (Morita et al., 2011; Romme Christensen et al., 2013; Tzartos et al., 2011).

### Type 1 Diabetes Mellitus (T1DM)

T1DM is an autoimmune disorder characterised by immune-mediated destruction of the pancreatic islet β cells. While the major cellular effectors of islet β cell destruction are generally considered as islet antigen-reactive T cells, a large body of evidence implicates B cells in this process and the pathogenesis of the disease (Smith et al., 2017).

The non-obese diabetic (NOD) mouse model of autoimmune diabetes exhibits an autoimmune insulitis. B cell deficient NOD mice exhibit suppression of insulitis, preservation of islet β cell function and protection against diabetes compared to NOD mice, indicating that B cells are essential for the development of diabetes in this model (Akashi et al., 1997; Noorchashm et al., 1997). Similar findings have been observed through use of anti-CD20 mediated B cell depletion, including reversal of established hyperglycaemia in a significant proportion of mice (Hu et al., 2007). Substantiating an important role for B cells in the pathogenesis of human T1DM, B cell depletion using rituximab results in partial preservation of islet β cell function in patients with newly diagnosed T1DM at 1 year (Pescovitz et al., 2009).

Studies with NOD mice suggest that islet autoantigen presentation by B cells to T cells is an important component of their pathogenic effect (Marino et al., 2012; Serreze et al., 1998). Alterations in peripheral blood B cell subsets have been identified in T1DM patients, including reduction in transitional B cells and an increase in plasmablast numbers (Parackova et al., 2017). In addition, circulating activated T follicular helper cells are increased in children with newly diagnosed T1DM and autoantibody positive at risk children (Viisanen et al., 2017).

The preclinical phase of T1DM is characterised by the presence if circulating islet autoantibodies, such as glutamic acid decarboxylase 65 (GAD65) and insulinoma antigen 2 (IA2) autoantibodies. The majority of children genetically at risk for T1DM with multiple islet autoantibody serocoversion subsequently progress to clinical diabetes (Ziegler et al., 2013). While these autoantibodies are predictive of development of T1DM, their precise pathogenic role is debated. Supporting evidence for their pathogenicity comes from studies in NOD mice where elimination of maternal transmission of autoantibodies from prediabetic NOD mice protects progeny from development of diabetes (Greeley et al., 2002). Notably, NOD mice deficient in activating Fc receptors for IgG (FcγR) are protected from spontaneous onset of T1DM (Inoue et al., 2007).

### Coeliac Disease and Dermatitis Herpetiformis

Coeliac disease is a chronic immune-mediated enteropathy against dietary gluten in genetically predisposed individuals (Lindfors et al., 2019). Adaptive immune responses play a key role in the pathogenesis of coeliac disease characterised by both antibody production towards wheat gliadin (IgA and IgG) and tissue transglutaminsase 2 enzyme (TG2) (IgA isotype), together with gluten-specific CD4⁺ T cell responses in the small intestine (van de Wal et al., 1998). The finding of TG2 as the primary autoantigen present in endomysium and the target for endomysial antibodies secreted by specific B cells (Dieterich et al., 1997) forms the basis of the primary coeliac antibody test used to support a diagnosis of coeliac disease with ^{˜}90-100% sensitivity/specificity (Rostom et al., 2005).

Multiple potentially pathogenic effects have been ascribed to coeliac disease autoantibodies (Caja et al., 2011) including of the IgA subclass, such as: interference with intestinal epithelial cell differentiation (Halttunen and Maki, 1999); promotion of retrotranscytosis of gliadin peptides to enable their entry into the intestinal muscosa to trigger inflammation (Matysiak-Budnik et al., 2008); increased intestinal permeability and induction of monocyte activation (Zanoni et al., 2006); and inhibition of angiogenesis via targeting of blood vessel TG2 in the lamina propria (Myrsky et al., 2008).

B cells specific for gluten and TG2 have been proposed to act as antigen-presenting cells to gluten-specific CD4⁺ T cells, with HLA-deamidated gluten peptide-T cell receptor interaction resulting in activation of both T and B cell, the latter differentiating into plasma cells with ensuing production of antibodies targeting gliadin and endogenous TG2 (du Pre and Sollid, 2015; Sollid, 2017).

While genetic association studies highlight a key role for CD4⁺ T cells in the pathogenesis of coeliac disease, integrative systems biology approaches have highlighted a significant role for B cell responses in coeliac disease (with disease SNPs significantly enriched in B-cell-specific enhancers) (Kumar et al., 2015).

Patients with active coeliac disease exhibit a marked expansion of TG2-specific plasma cells within the duodenal mucosa. Further increases in extracellular IgM and IgA are evident in the lamina propria and epithelial cells in response to gluten, consistent with an active immunoglobulin response within the small intestinal mucosa (Lancaster-Smith et al., 1977). Notably TG2-specific IgM plasma cells have been described in coeliac disease, which could exert pathogenic effects via their ability to activate complement to promote inflammation. Indeed, subepithelial deposition of terminal complement complex has been observed in untreated and partially treated (but not successfully treated) patients with coeliac disease, correlating with serum levels of gluten-specific IgM and IgG (Halstensen et al., 1992).

Dermatitis herpetiformis is an itchy blistering skin disorder regarded as the cutaneous manifestation of coeliac disease (Collin et al., 2017). It is characterised by granular IgA deposits in the dermal papillae of uninvolved skin (Caja et al., 2011). Patients with dermatitis herpetiformis exhibit autoantibodies against epidermal TG3, which are gluten-dependent, and respond slowly to a gluten-free diet (Hull et al., 2008). Its pathogenesis is thought to involve active coeliac disease in the intestine resulting in the formation of IgA anti-TG3 antibody complexes in the skin.

Notably B cell depletion with rituximab has resulted in complete clinical and serological remission in a case of refractory dermatitis herpetiformis (Albers et al., 2017). Similarly, rituximab has resulted in dramatic clinical improvement in a mixed case of symptomatic coeliac disease and Sjogren's syndrome (Nikiphorou and Hall, 2014).

### Psoriasis

Psoriasis is a chronic, immune-driven disease primarily affecting the skin and joints (Greb et al., 2016). Pathophysiologically, psoriasis involves components of innate and adaptive immunity, particularly involving T cell (specifically T_{H}17 cell) signalling, dendritic cells and keratinocytes (Greb et al., 2016).

Analysis of psoriatic arthritis synovium has revealed frequent ectopic lymphoid neogenesis which can drive local antigen-driven B cell development, which notably regressed with treatment (Canete et al., 2007). Critically these tertiary lymphoid structures triggered by persistent inflammation contain highly organised follicles, segregated B cell and T cell zones and follicular dendritic cell networks providing the substrate for a germinal centre response to support local (aberrant) adaptive immune responses against locally displayed antigens, including autoreactive lymphocyte clone cell survival and pathogenic immunoglobulin production (Canete et al., 2007; Pipi et al., 2018).

Psoriasis has recently been identified to be associated with several serum autoantibodies, including IgG against LL37 (cathelicidin) and ADAMTSL5 (a disintegrin and metalloprotease domain containing thrombospondin type 1 motif-like 5), whose levels correlate with psoriasis clinical severity and reflect disease progression over time (Yuan et al., 2019). Notably expression of these autoantigens is reduced by effective therapy targeting IL-17 or TNF-α, suggesting positive regulation and feedforward induction by psoriasis disease-related pro-inflammatory cytokines (Fuentes-Duculan et al., 2017). Other autoantibodies identified such as those against anti-α6-integrin have been proposed to contribute to induction of a chronic wound healing phenotype (Gal et al., 2017). Analysis of total circulating immunoglobulins in psoriasis has revealed elevated total IgA, but not total IgG or IgM (Kahlert et al., 2018). Supporting this increase, an elevation in plasmablast levels in psoriasis has also been noted (Kahlert et al., 2018).

Analysis of peripheral blood lymphocyte subsets has revealed an expansion in circulating activated B cells and T_{FH} cells together with elevated serum IL-21 in psoriasis compared to healthy donors; notably the levels of each of these correlated positively with psoriasis severity (Niu et al., 2015). Substantiating the functional importance of this, circulating T_{FH} cells from psoriasis patients exhibit signs of activation and produce higher levels of cytokines, with significant reduction in these on treatment. Moreover, psoriasis lesions exhibit extensive T_{FH} infiltration (Wang et al., 2016b). IL-10 producing regulatory B cells (i.e. B10 cells) have been found to be reduced in psoriasis, exhibit impaired activity and inversely correlate with IL-17 and IFN-γ producing T cells (Mavropoulos et al., 2017).

There are reports of B cell depletion using rituximab inducing de novo psoriasis skin lesions (Dass et al., 2007), although this is debated (Thomas et al., 2012), but improved arthritis (Jimenez-Boj et al., 2012), highlighting the complex role of B cells in the pathogenesis of the disease and the importance of non-canonical B cell function (i.e. beyond autoantibody production) including but not limited to cytokine production and antigen presentation to influence autoreactive T cells (Hayashi et al., 2016; Yoshizaki et al., 2012).

### The Idiopathic Inflammatory Myopathies (IIM), Including Dermatomyositis (DM) and Polymyositis (PM)

DM and PM are inflammatory myopathies typically resulting in symmetrical proximal myopathy that differ in clinical features, pathology and clinical response/prognosis (Findlay et al., 2015). DM is characterised by skin lesions and (usually except in amyopathic cases) inflammation of skeletal muscle. PM is traditionally the term ascribed to idiopathic inflammatory myopathy which is neither DM nor sporadic inclusion body myositis (Findlay et al., 2015). Other subtypes of IIM recognised include necrotising autoimmune myositis and overlap syndrome (Dalakas, 2015).

Supporting a role for B cells, IIMs are associated with autoantibody production, both myositis-specific and myositis-associated, useful clinically in diagnosis, including for DM (Anti-MDA-5, anti-Mi-2, anti-TIF-1, anti-NXP-2), PM (anti-synthetase antibodies), necrotising autoimmune myositis (anti-HMGCR, anti-SRP) and inclusion body myositis (anti-cN1A) (Dalakas, 2015). Notably autoantibody levels in patients with myositis have been shown to reduce with B cell depletion and correlate with changes in disease activity (Aggarwal et al., 2016).

DM is thought to be substantially humorally mediated through pathogenic antibody-mediated complement activation on endothelial cells resulting in necrosis and ischaemia and muscle fibre destruction (Kissel et al., 1986), i.e. a complement-mediated microangiopathy. Indeed, ectopic lymphoid structures have been identified in skeletal muscle of patients with DM, including evidence of germinal centres with dark/light zone organisation and molecular evidence of in situ B cell differentiation (Radke et al., 2018). PM and inclusion body myositis have traditionally been regarded as primarily CD8⁺ cytotoxic T cell-mediated disorders, however abundant enrichment of plasma cells has been identified in muscle biopsies from patients with these disorders and associated high expression of immunoglobulin transcript (Greenberg et al., 2005). Further supporting a local B cell antigen-specific response in PM and inclusion body myositis is the finding of affinity maturation (encompassing somatic mutation, class switching and oligoclonal expansion) within IgH chain gene transcripts of local B cells and plasma cells in patients but not in control muscle tissue (Bradshaw et al., 2007). Similar B cell clonal diversification has been noted in DM consistent with an antigen-driven chronic B cell response in inflamed muscle (McIntyre et al., 2014).

Serum levels of BAFF (B cell-activating factor belonging to the tumour necrosis factor family), a critical factor in B cell survival and maturation, is significantly elevated in DM in association with increased expression of BAFF in the perifascicular area of skeletal muscle of patients versus normal controls (Baek et al., 2012). Notably expression of BAFF receptors have been co-localised to or in the vicinity of plasma cells and B cells in patients with myositis with a correlation between the number of cells expressing BAFF receptors and plasma cell frequency, particularly those expressing anti-Jo-1 or anti-Ro52/Ro60 autoantibodies, consistent with local BAFF-driven differentiation of plasma cells in myositis (Krystufkova et al., 2014). Supporting a functional role for these changes, BAFF pathway expression is positively correlated with measures of disease activity in idiopathic inflammatory myopathies (Lopez De Padilla et al., 2013).

Supporting a key pathogenic role for B cells in the idiopathic inflammatory myopathies, refractory skin rashes have shown improvement in response to B cell depletion using rituximab (Aggarwal et al., 2017), with evidence of some clinical response in patients with DM or PM (Mok et al., 2007; Oddis et al., 2013; Sultan et al., 2008).

Highlighting a specific role for T-B cell interaction and CD4⁺ T cell help for B cell responses in DM, alteration in circulating T_{FH} cell subsets have been observed skewed towards subtypes favouring B cell help to promote immunoglobulin production via IL-21 (Morita et al., 2011). Notably such circulating T_{FH} cells promote differentiation of naïve B cells to plasmablasts (Morita et al., 2011).

### Interstitial Lung Disease (ILD)

ILD encompass a complex and heterogeneous set of disorders, including idiopathic pulmonary fibrosis (IPF), hypersensitivity pneumonitis, drug-associated ILD, sarcoidosis and ILD associated with connective tissue disorders and familial/other syndromes (Wallis and Spinks, 2015).

Supporting a role for B cells in driving the progression of ILD, use of rituximab in patients with severe, progressive non-IPF ILD refractory to conventional immunosuppression shows evidence of improvement in lung capacity and stabilisation of diffusing capacity of carbon monoxide (Keir et al., 2012; Keir et al., 2014). Striking clinical improvement has also been reported in response to rituximab in a case of severe refractory hypersensitivity pneumonitis (Lota et al., 2013), a condition associated with germinal cell formation in bronchus-associated lymphoid tissue (Suda et al., 1999). Favourable responses to B cell depletion have also been reported in severe cases of ILD associated with anti-synthetase (Sem et al., 2009) and systemic sclerosis (Sari et al., 2017).

IPF is associated with circulating IgG autoantibodies (Feghali-Bostwick et al., 2007), with morphological evidence of microvascular injury in association with IgG, IgM and IgA deposition within septal microvasculature suggesting antibody-mediated microvascular injury (Magro et al., 2006). Autoantigens identified include annexin 1, with evidence of significant elevation in autoantibody targeting annexin 1 during acute exacerbations of IPF (Kurosu et al., 2008) suggesting a potential role in these episodes. Notably immune complex formation between antigens and immunoglobulin—a potent trigger of inflammation and secondary injury—are present in IPF in the circulation (Dobashi et al., 2000), lung parenchyma (with complement deposition) (Xue et al., 2013) and from bronchoalveolar lavage.

Histology of lungs of patients with IPF has also identified abnormal B cell aggregates including germinal centre formation, particularly close to fibroproliferative areas (Campbell et al., 1985; Marchal-Somme et al., 2006). Moreover, IPF is associated with elevated circulating and local CXCR13—a CD4⁺ T cell-derived chemokine promoting pathological B cell trafficking and formation of ectopic lymphoid-like structures and elevated in several autoantibody-mediated disorders—and this elevation correlates with exacerbations and poor outcomes suggesting a pathogenic role for CXCR13 and B cells in IPF (Vuga et al., 2014; Yoshitomi et al., 2018). Moreover, the circulating plasmablast pool is expanded in IPF, with evidence of greater antigen differentiation of circulating B cells and significantly increased plasma levels of BLyS (B lymphocyte stimulating factor) a key promoter of B cell survival and differentiation, with patients displaying the highest levels of BLyS also those with the lowest 1-year survival rates (Xue et al., 2013).

In the setting of IPF, evidence exists supporting a role for targeting pathogenic autoantibody using therapeutic plasma exchange and rituximab to alleviate acute respiratory exacerbations in critically ill patients with IPF which can otherwise be fatal within days (Donahoe et al., 2015). Notably plasma exchange was associated with a reduction in anti-Hep-2 autoantibodies in patients responding to treatment (Donahoe et al., 2015).

Inflammatory Bowel Disease (IBD)—Ulcerative Colitis (UC) and Crohn's Disease (CD) UC is an idiopathic IBD characterised by inflammation of the colon and rectum.

UC is associated with an expanded circulating plasmablast subset of B cells together with elevated serum IgG (Wang et al., 2016a). Notably, inflammatory markers (CRP and ESR) correlate positively with levels of plasmablasts and serum IgG levels. Conversely, treatment with mesalazine lowers plasmablast levels in UC (Wang et al., 2016a).

UC is associated with autoantibody formation mainly antineutrophil cytoplasmic antibodies (ANCA) and anti-goblet cell antibodies with the latter considered potentially specific and both aiding differentiation from CD in early cases (Conrad et al., 2014). Underlining a pathogenic role for autoantibodies in UC is the finding of complement activation in relation to epithelial-bound IgG (Brandtzaeg et al., 2006). The known substantial infiltration of the colon with B cells and plasma cells in UC, as in CD, provides a local source for these (Cupi et al., 2014).

Highlighting a role for altered T follicular regulatory and T_{FH} subsets, key T cell subsets whose balance regulates B cell responses, patients with UC exhibit an increase in circulating T_{FH} cells but lower T follicular regulatory cell levels, in conjunction with elevated IL-21 and reduced IL-10 (Wang et al., 2017). Notably, serum IL-21 level and circulating T_{FH} cell level positively correlate with clinical severity score and systemic inflammatory markers, with the converse holding for levels of circulating T follicular regulatory (T_{FR}) cells and IL-10 (Wang et al., 2017). This imbalance in the T_{FR}/T_{FH} ratio has been observed also in other canonical B cell driven pathogenic immunoglobulin-mediated disorders such as myasthenia gravis.

While B cell depletion with rituximab has not proven effective in steroid-unresponsive moderate UC in a clinical trial setting (Leiper et al., 2011), colon-resident plasma cells have been shown to be unaffected by this therapy, suggesting failure to target this B cell cellular/anatomic compartment may contribute to the observed lack of efficacy (Uzzan et al., 2018). Notably the pathogenic effects of plasma cells may not be limited to pathogenic autoantibody production—both UC and CD are characterised by mucosal accumulation of IgA⁺ plasma cells expressing granzyme B, a serine protease induced by IL-21 in B cells and linked to induction of apoptosis after cytotoxic cellular attack (Cupi et al., 2014; Hagn et al., 2010).

CD is characterised by transmural inflammation of the gastrointestinal tract and any affect any part of it and, like UC, exhibits a significant increase in plasma cells in the intestinal lamina propria as a source of both IgG and monomeric IgA (Uzzan et al., 2018). Notably, IgG plasma cells correlate with the severity of intestinal inflammation (Buckner et al., 2014). Furthermore, B cells are seen to localise around a key pathological hallmark of CD, intestinal granulomas (Timmermans et al., 2016). Analysis of circulating class switched memory B cells in CD reveals increased levels of somatic hypermutation consistent with chronic stimulation (Timmermans et al., 2016). Notably, alterations in the peripheral B cell compartment improve with effective treatment of inflammation through targeting of TNF-α (Timmermans et al., 2016).

As with UC, patients with CD show abnormal B cell responses in the form of detectable (IgG/IgA) auto- or anti-microbial antibodies, including against Saccharomyces cerevisiae antibodies (ASCA) and neutrophils (ANCA), with serological markers predictive of disease prior to diagnosis (Quinton et al., 1998; van Schaik et al., 2013), as well as of risk of recurrence post-surgical resection (Hamilton et al., 2017). Underlining the pathogenic potential of these, autoantibodies against the cytokine granulocyte-macrophage colony-stimulating factor (GM-CSF) are produced by lamina propria cells and have been associated with stricturing behaviour, which may reflect their ability to reduce neutrophil function, and increased intestinal permeability (Jurickova et al., 2013).

Highlighting a role for T cells contributing to the observed B cell phenotype of CD, circulating T_{FH} cells are increased in patients with CD versus controls (Wang et al., 2014b).

### Autoimmune Thyroid Disease (AITD), Including Graves' Disease and Hashimoto's Thyroiditis

AITD is an organ-specific autoimmune disorder characterised by breakdown of self-tolerance to thyroid antigens. Genome-wide association studies have revealed a role for genetic variants in B cell signalling molecules in the development of AITD (Burton et al., 2007), including FCRL3 (Chu et al., 2011b) and BACH2 involved in B cell tolerance, maturation and class switching (Muto et al., 2004).

Pathologically, AITD exhibits intense lymphocyte accumulation in the thyroid gland, including B cells at the time of diagnosis (notably in Hashimoto's thyroiditis) and production of anti-thyroid antibodies (Zha et al., 2014). Patients with recent-onset AITD display thyroid antigen-reactive B cells in the peripheral blood which are no longer anergic but express the activation marker, CD86, consistent with activation of these cells to drive autoantibody production (Smith et al., 2018).

Graves' disease is characterised by production of pathognomonic agonistic anti-thyrotropin receptor IgG autoantibodies (found in 80-100% of untreated patients) which mimic TSH and stimulate thyroid hormone overproduction and thyroid enlargement (Singh and Hershman, 2016). Patients with Graves' disease exhibit elevated transitional and pre-naïve mature B cells in peripheral blood, with levels positively correlating with those of free thyroxine (Van der Weerd et al., 2013). Consistent with a B cell-driven pathophysiological process and potentially contributing to the expansion of these B cell populations, the serum levels of BAFF (B lymphocyte activating factor)—a key factor promoting B cell autoantibody production by increasing B cell survival and proliferation—are raised in patients with Graves' disease and fall in response to methylprednisolone treatment (Vannucchi et al., 2012). Hyperthyroidism itself promotes plasma cytogenesis to increase plasma cells in the bone marrow (Bloise et al., 2014). B cell depletion using anti-mouse monoclonal CD20 antibody in a mouse immunisation model of model of Graves' disease is effective in suppressing anti-TSHR antibody generation and hyperthyroidism given before immunisation or 2 weeks later (Ueki et al., 2011). Mirroring this, rituximab has demonstrated efficacy clinically in Graves' orbitopathy (Salvi et al., 2013).

In Hashimoto's thyroiditis, B cells generate autoantibodies against thyroglobulin (>90% patients) and thyroid peroxidase which lead to apoptosis of thyroid follicular cells via antibody-dependent cell-mediated cytotoxicity. Plasma cell accumulation has been noted in thyroidectomy specimens from patients with Hashimoto's thyroiditis in association with foci of thyroid follicular destruction (Ben-Skowronek et al., 2013).

T_{FH} cells, which regulate (auto-)antibody production by B cells, are found to be expanded in the circulation of patients with AITD, with a positive correlation with autoantibody titres and also levels of free thyroid hormone in Grave's disease; moreover, these cells reduce with therapy and have been found to be enriched in thyroid tissue from patients with Hashimoto's thyroiditis (Zhu et al., 2012).

### Autoimmune Uveitis and Autoimmune Retinopathy

Uveitis refers to inflammation of the tissues of the eye, ranging from the anterior chamber which includes the iris and ciliary body, to the vitreous, to posterior structures (retina or choroid) (Smith et al., 2016). Notably uveitis is observed in association with systemic autoimmune and inflammatory diseases, such as seronegative spondyloarthritis, IBD, psoriatic arthropathy, Behcet's disease, rheumatoid arthritis, juvenile idiopathic arthritis, in addition to infectious and other aetiologies (Selmi, 2014). Autoimmune uveitis is therefore a collection of disorders in which there is loss of ocular immune privilege and which can be associated with disease affecting other tissues.

Autoimmune retinopathy is associated with progressive loss of visual acuity in association with anti-retinal antibodies (Grange et al., 2014). Autoantibodies against multiple retinal proteins have been identified, including retinal specific proteins such as recoverin localised in photoreceptors and α-enolase (Ren and Adamus, 2004), the former also described in cancer-associated retinopathy. Anti-recoverin antibodies are able to penetrate retinal layers to promote apoptotic photoreceptor cell death (Adamus, 2003). Notably patients with autoimmune retinopathy exhibit altered peripheral mature B cell memory subsets, including evidence of activation of naïve memory B cells and altered isotype profile (Stansky et al., 2017).

Murine models of autoimmune uveitis suggest T helper cells, specifically T_{H}1 and T_{H}17 cells as being important effectors. However, B cells are felt to play in important pathogenic role through uveal antigen presentation and subsequent activation of T cells (Prete et al., 2016), inflammatory cytokine production and support of T cell survival (Smith et al., 2016). Antigens involved are thought to include melanocyte components or tyrosinase or related proteins including recoverin, rhodopsin and retinal arrestin (Prete et al., 2016). In addition to direct cell toxicity described above for retinal autoantibodies, autoantibodies in autoimmune uveitis may exert pathogenic effects through formation of antigen-antibody immune complexes to trigger innate immune mechanisms or complement activation via the classical pathway (Smith et al., 2016). As a corollary, mice deficient in complement (C3) develop less severe experimental autoimmune uveitis than controls (Read et al., 2006).

Evidence for involvement of B cells in autoimmune uveitis include: the presence of B cells in the intra-ocular inflammatory infiltrate and vitreous immunoglobulin (Godfrey et al., 1981; Nguyen et al., 2001), remission of ocular disease in association with onset of combined variable immunodeficiency (CVID, a primary immunodeficiency syndrome associated with impaired B cell differentiation and hypogammaglobulinaemia) (Amer et al., 2007), elevation of serum BAFF in autoimmune disease with co-existing uveitis (Gheita et al., 2012) and the response to rituximab (described below).

Highlighting a role for B cell mediated homeostatic regulation of T cell function that is perturbed in an experimental model of uveitis, tonic inhibition of T cell trafficking by B cell derived peptide release (PEPITEM) is lost, facilitating T cell recruitment to promote chronic tissue injury (Chimen et al., 2015). Furthermore, IL-35 promoted induction of regulatory B cells is protective in experimental autoimmune uveitis, in part through inhibition of pathogenic T_{H}17 and T_{H}1 cells whilst enhancing expansion of Treg cells (Wang et al., 2014a).

Notably, B cell depletion with rituximab has shown efficacy in stabilising and/or improving visual acuity in patients with autoimmune retinopathy (Maleki et al., 2017) and autoimmune uveitis and scleritis (Hardy et al., 2017; Pelegrin et al., 2014).

### Mixed Connective Tissue Disease (MCTD) and Undifferentiated Connective Tissue Disease (UCTD)

MCTD is a systemic autoimmune disorder characterised by the presence of antibodies to U1-RNP (U1-ribonuclear protein).

In addition to acting as a serological hallmark for MCTD diagnosis, anti-U1 RNP autoantibodies are thought to play a central pathogenic role (Tani et al., 2014), including binding to pulmonary artery endothelial cells (that may promote pulmonary hypertension via triggering of endothelial cell inflammation) (Okawa-Takatsuji et al., 2001). Further evidence strongly suggesting a role for this antibody in the pathogenesis of MCTD comes from studies involving immunisation of mice with antigenic peptide of the U1-70-kd subunit of the U1 snRNP in which induction of anti-RNP antibodies and MCTD-like autoimmunity including interstitial lung disease resulted (Greidinger et al., 2006). Autoantibodies are also thought to promote tissue injury in MCTD via immune complex formation and complement activation (Szodoray et al., 2012).

Beyond U1-RNP, other findings highlighting altered humoral adaptive immunity in MCTD are the frequent presence of other autoantibodies (e.g. ANA), hypergammaglobulinaemia and polyclonal B cell hyperreactivity and activation (Hajas et al., 2013).

Consistent with altered B cell homeostasis in MCTD, analysis of peripheral B cell subsets reveals altered numbers of transitional cells, naïve B cells and memory B cells, together with increased plasma cell number correlating with levels of anti-U1-RNP (Hajas et al., 2013). Furthermore, in common with other connective tissue disorders, abnormalities of bone marrow are reported including increase in plasma cell number in association with lymphoid aggregates (Rosenthal and Farhi, 1989).

Supporting an important role for B cells in the pathology of MCTD, B cell depletion using rituximab has been shown to stabilise pulmonary function in patients with associated interstitial lung disease (Lepri et al., 2016). Further supporting a role for pathogenic immunoglobulin and/or immune complexes in MCTD, plasmapheresis (Seguchi et al., 2000), immunoadsorption (Rummler et al., 2008) including combined with anti-CD20 therapy (Rech et al., 2006) has reported efficacy.

Highlighting a T cell component likely to contribute to the pathogenesis of MCTD, levels of circulating Tregs are reduced and even lower in patients with active disease.

UCTD describes a group of unclassifiable systemic autoimmune diseases which overlap with serological and clinical features of definite connective tissue diseases (CTD), e.g. SLE, systemic sclerosis, DM, PM, MCTD, rheumatoid arthritis and Sjogren's syndrome, but which do not fulfil criteria for classification into a specific CTD (Mosca et al., 2014). Notably a significant proportion of these patients go on to evolve into a defined CTD (Mosca et al., 2014). Patients often exhibit positive anti-nuclear antibodies (ANA).

Patients with UCTD have been shown to exhibit significantly increased expression of the activation marker CD86 on circulating B cells with nominal but non-statistically significant increases in circulating plasma cells and T_{FH} cells (Baglaenko et al., 2018). Highlighting a T cell component to the disease, patients with UCTD show lower levels of circulating CD4⁺CD25⁺Foxp3⁺ regulatory T cells (Tregs) together with elevated INF-γ production (Szodoray et al., 2008).

### Autoimmune Connective Tissue Disease Such as Systemic Lupus Erythematosus (SLE); Discoid Lupus Erythematosus (DLE)

SLE is a multisystem archetypal autoimmune connective tissue disease (CTD) predominantly affecting women with a predilection for affecting the kidneys, joints, central nervous system and skin and the presence of autoantibodies against nucleic acids and nucleoproteins (Kaul et al., 2016). SLE is associated with a number of autoantibodies, some of which antedate the clinical onset by several years, such as IgG/IgM antiphospholipid antibodies, antinuclear antibodies (ANA) and others (McClain et al., 2004). Additional antibody targets and disease associations include: C1q, dsDNA and Smith (Sm) in lupus nephritis, Ro (SSA, Sjogren syndrome-related antigen) and La (SSB) in secondary Sjogren syndrome and cutaneous lupus, U1-RNP and Ro in interstitial lung disease, prothrombin and β2 glycoprotein 1 in antiphospholipid syndrome (Kaul et al., 2016). Many of these autoantibodies are regarded as pathogenic, largely through the formation of immune complexes and deposition, e.g. in renal glomeruli and skin, to induce immune activation via complement activation or via Fc receptors. Immune complexes can promote B cell and dendritic cell activation leading to cytokine production (e.g. IFN-α) (Means and Luster, 2005), in addition to activating neutrophils via FcγRIIA to promote reactive oxygen species (ROS) and chemokine release inducing tissue damage (Bonegio et al., 2019).

Beyond autoantibody production indicating a breakdown of self-tolerance in B cells, multiple lines of evidence implicate B cells as major contributors to the pathophysiology of SLE. Patients with active lupus exhibit defects in central and peripheral B cell tolerance which would facilitate the survival and activation of autoreactive B cells (Jacobi et al., 2009; Yurasov et al., 2005). B cell hyperactivity and plasmacytoid dendritic cell interaction together with RNA-containing immune complexes serves to promote further B cell expansion (Berggren et al., 2017).

A mouse model exhibiting SLE-like pathology spontaneously forms germinal centres with increased plasma cell number and lowered threshold for B cell activation and impaired elimination of autoreactive B cells (Kil et al., 2012). Lupus prone mice display expansion of antigen-activated marginal zone (MZ) B cells which migrate to lymphoid follicles to engage with CD4⁺ T cells to promote autoantibody production, consistent with a breach in follicular exclusion (Duan et al., 2008; Zhou et al., 2011).

B cell-T cell interaction is a critical contributor to the pathogenesis of SLE, including via activation of autoreactive B cells by T cell subsets and promotion of high-affinity autoantibodies from germinal centres supported by T_{FH} cells. Murine models of lupus demonstrate abnormal T_{FH} expansion and dysregulated germinal centre reactions correlating with autoantibody level (Kim et al., 2015), driven in part through elevated IL-21 (Bubier et al., 2009) and ICOS-dependent (Mittereder et al., 2016) signalling released/mediated by T_{FH} cells. Similarly, findings from patients with SLE indicate increased levels of active T_{FH} cells correlating with autoantibody titre, severity of organ involvement by disease and plasma cell number with evidence of downregulation in response to corticosteroids (Feng et al., 2012; Simpson et al., 2010), Notably these circulating T_{FH} cells are phenotypically similar to those present in germinal centres, correlate with circulating plasmablast levels and promote B cell differentiation to IgG-secreting plasma cells in vitro (Zhang et al., 2015).

Further supporting a role for B cells as key mediators of disease in SLE are observations of clinical efficacy with B cell depletion using rituximab in refractory patients (Iaccarino et al., 2015), including lupus nephritis except in rapidly progressive crescentic cases (Davies et al., 2013) and neuropsychiatric lupus (Tokunaga et al., 2007). Notably, more rapid memory B cell and plasmablast repopulation post-rituximab are associated with earlier disease relapse (Vital et al., 2011). Notably rituximab use in SLE is also associated with altered cytokine levels and T cell phenotypes beyond simple B cell depletion highlighting an effect on the latter as a likely contributor to its efficacy (Tamimoto et al., 2008). Supporting a pathogenic role for autoantibodies in lupus, autoantibody removal using immunoadsorption has provided clinical benefits in refractory disease (Kronbichler et al., 2016).

DLE, the most common form of chronic cutaneous SLE, has been associated with polyclonal B cell activation (Wangel et al., 1984), together with increased numbers of B cells in skin (Hussein et al., 2008) which can promote skin fibrosis via cytokine release, further enhanced by BAFF (Francois et al., 2013) and a predominance of T cells (Andrews et al., 1986). Notably abnormalities in circulating B cells in discoid lupus similar to that of SLE have been identified, including a correlation with clinical disease criteria (Kind et al., 1986; Wouters et al., 2004). Furthermore, B cell depletion using rituximab has proven effective for cutaneous manifestations of SLE (Hofmann et al., 2013) and DLE (Quelhas da Costa et al., 2018).

### Immune-Mediated Inflammatory Disease (IMID) Such as Scleroderma (SS, Systemic Sclerosis), Rheumatoid Arthritis and Sjogren's Disease

SS is an immune-mediated inflammatory disease typified by fibrosis of the skin and internal organs together with a vasculopathy (Denton and Khanna, 2017).

SS is associated with autoantibody formation including anti-centromere, anti-Scl-70, anti-RNA polymerase III (and other ANA), with strong relation to disease presentation/internal organ involvement and outcome (Nihtyanova and Denton, 2010). Evidence of autoantibodies as pathogenic drivers of the complications of SS include documentation of functional autoantibodies targeting platelet-derived growth factor receptor (PDGFR) which promote PDGFR stimulation and collagen and alpha-smooth muscle actin expression to support a pro-fibrotic phenotypic transition of fibroblasts (Gunther et al., 2015). Other functional autoantibodies detected in SS include against those targeting Angiotensin II type 1 receptor (AT1R) and endothelin type A receptor (ETAR), promoting agonistic activity at these receptors and strongly predictive of severe SS complications and mortality (Becker et al., 2014; Riemekasten et al., 2011).

SS is associated with polyclonal B cell activation and increased serum IgG (Famularo et al., 1989). Notably circulating B cells from patients with SS overexpress CD19 consistent with heightened intrinsic B cell activation which is expected to promote autoantibody production (Tedder et al., 2005). Increased activation markers are also seen specifically in the memory B cell pool in SS, with enhanced ability to produce IgG in vitro (Sato et al., 2004). Notably the diffuse cutaneous variant of SS has been associated with an expanded circulating class-switched memory B cell population (Simon et al., 2016). Further supporting an alteration in B cell homeostasis in SS is the finding of an elevation in serum levels of key cytokines and B cell factors involved in regulating B cell activation, survival or homing, including IL-6, BAFF and CXCL13 (Forestier et al., 2018). Notably BAFF is upregulated in affected skin of patients with SS, with increases in serum levels of BAFF correlating with new onset or exacerbation of organ involvement and conversely reduction in serum BAFF observed with skin lesion regression (Matsushita et al., 2006).

Pathologically, cutaneous lesions have been shown to include cellular infiltrates containing plasma cells (Fleischmajer et al., 1977). Furthermore, highlighting a role for T cell regulators of autoantibody production by B cells, T cells possessing a T_{FH} phenotype including expression of ICOS are seen to infiltrate cutaneous lesions of SS and correlate with both dermal fibrosis and disease status clinically (Taylor et al., 2018). As a corollary, anti-ICOS antibody or IL-21 neutralisation administered to a murine model of SS-GVHD (graft-versus-host-disease) reduces dermal inflammation and/or fibrosis (Taylor et al., 2018).

Clinically, B cell depletion using rituximab has exhibited a beneficial effect on pulmonary function (or stabilisation) and improvement of skin thickening in SS associated with interstitial lung disease (Daoussis et al., 2017; Jordan et al., 2015).

### Rheumatoid Arthritis (RA)

RA is associated with a large number of autoantibodies, most well described being rheumatoid factors and anticitrullinated protein antibodies (ACPA) but including others such as anti-carbamylated protein antibodies and anti-acetylated protein antibodies. As with SLE, the presence of these autoantibodies can antedate clinical expression by years and also associate with radiographic disease progression (Derksen et al., 2017).

ACPA antibodies include IgG, IgA and IgM and given the presence of citrullinated protein in synovial fluid from inflamed RA joints, suggests that ACPA could bind these (Derksen et al., 2017). The collagen-induced arthritis mouse model develops antibodies against both CII and cyclic citrullinated peptide early after immunisation, with administration of murine monoclonal antibodies against citrullinated fibrinogen enhancing arthritis and binding inflamed joint synovium (Kuhn et al., 2006). Notably, the Fab-domain of ACPAs display a high abundance of N-linked glycans which may alter its properties to promote specific effector functions to ACPA IgG, such as binding of immune cells (Hafkenscheid et al., 2017). Immune complexes containing ACPA and citrullinated fibrinogen can stimulate TNF production via binding of Fcγ receptors on macrophages (Clavel et al., 2008), including macrophages derived from synovial fluid of patients (Laurent et al., 2011). Complement activation through autoantibodies is also a likely mechanism of pathogenicity in RA, supported by evidence of enhanced complement activation from synovial fluid of RA patients and the ability of ACPA to activate complement via both the classical and alternative pathways (Trouw et al., 2009). Pathogenic autoantibodies have also been linked to RA-associated bone loss through IL-8 mediated enhancement of osteoclast differentiation (Krishnamurthy et al., 2016).

RA is associated with defective central and peripheral B cell tolerance, contributing to an excess of autoreactive B cells in the mature naïve B cell subpool, increased proportion of polyreactive antibodies recognising immunoglobulins and cyclic citrullinated peptides (Samuels et al., 2005b). Notably despite immunosuppressive therapy in RA, post-treatment frequency of autoreactive mature naïve B cell clones remains elevated consistent with primary defective early B cell tolerance and a limited ability of current therapeutics to target this (Menard et al., 2011).

Serum levels of BAFF are high in early RA and correlate with titres of IgM rheumatoid factor and anti-cyclic citrullinated peptide autoantibody, as well as with joint involvement; furthermore, levels of BAFF improve in parallel with clinical severity and autoantibody levels in response to methotrexate therapy (Bosello et al., 2008). Notably a cytokine environment conducive to B cell activation and survival has been discerned in very early RA, specifically elevation in BAFF and APRIL (a proliferation-inducing ligand, involved in class-switch recombination and plasma cell differentiation and survival) levels including enrichment in synovial fluid, suggesting a primary role in disease (Moura et al., 2011). Pathologically, RA articular synovium demonstrates infiltration of plasma cells, positively correlating with synovial fluid levels of APRIL (Dong et al., 2009).

Supporting a key role for T-B interactions in activating autoreactive B cells, T cell promotion of extra-follicular B cell responses as an alternative means of B cell activation via Toll-like receptors amplifies autoantibody production through CD40L and IL-21 signalling (Sweet et al., 2011). Moreover, mice deficient in CXCR5 on T cells are resistant to development of CIA, exhibiting impaired germinal centre formation and failing to mount an IgG1 antibody response to CII (Moschovakis et al., 2017).

Patients with RA show an expansion in peripheral circulating T_{FH} cells, correlating with autoantibody titres; notably circulating plasmablast levels in RA correlate with clinical disease activity and markers of inflammation (CRP, ESR) (Nakayamada et al., 2018). In this context plasmablasts may function to present antigen to T cells and promote T cell differentiation, in addition to antibody secretion, thus perpetuating joint inflammation (Nakayamada et al., 2018). Notably, T_{FH} cells have also been identified within RA synovium as part of the immune infiltrate (Chu et al., 2014), together with regulatory T cells (Tregs) (Penatti et al., 2017). Highlighting a potential pathogenic consequence of the latter, Tregs appear functionally compromised in RA, an effect improved following anti-TNF-α therapy (Ehrenstein et al., 2004). Importantly, while CD4⁺CD25⁺Foxp3⁺ Tregs are enriched in inflamed RA synovium, they appear less functional indicating a poorer ability to mediate immune tolerance (Sun et al., 2017). A potential mechanism underlying this observation is that of B cell-derived IFN-γ mediated suppression of Treg differentiation, shown to promote autoimmune experimental arthritis in mice (Olalekan et al., 2015).

B cell depletion in RA using rituximab significantly improves symptoms in RA (Edwards et al., 2004), including in patients refractory to anti-TNF-α therapy (Cohen et al., 2006). Rituximab in RA is more effective in seropositive cases (i.e. patients exhibiting ACPA and RF); moreover, positive clinical responses correlate with significant reductions in autoantibodies in parallel with inflammatory markers (Cambridge et al., 2003), as well as the extent of B cell depletion (Vancsa et al., 2013). Autoantibody depletion using immunoadsorption has also proven efficacious in refractory RA (Furst et al., 2000), likely in part to relate to removal of immune complexes and potentially due to removal of complement components (Kienbaum et al., 2009).

### Sjogren's Syndrome (SjS; Sjorgen's Disease)

SjS is a systemic autoimmune disorder which primarily results in inflammation and destruction of exocrine glands by inflammatory infiltrates and IgG plasma cells (especially salivary and lacrimal) with ensuring tissue destruction, but can lead to systemic disease characterised by peri-epithelial infiltration by lymphocytes and immune complex deposition (Brito-Zeron et al., 2016). The latter contain T cells, B cells and plasma cells (Hansen et al., 2007). Systemic involvement, e.g. renal disease, is also characterised by marked enrichment of these cells, especially plasma cells (Jasiek et al., 2017).

SjS syndrome is associated with a number of autoantibodies against autoantigens including Ra, La, Fc fragment of IgG and muscarinic M3 receptors. IgG autoantibodies targeting M3 from patients with SjS have been shown to exert an anti-secretory effect in both mouse and human acinar cells, an impact expected to damage salivary production and contribute to the xerostomia (dry mouth) observed in patients (Dawson et al., 2006).

Ectopic formation of germinal centres is recognised in salivary glands in SjS, with B cell-T cell interactions within the germinal centre important to disease pathogenesis and B cell dysregulation (Pontarini et al., 2018). Other evidence for B cell hyperactivity in SjS includes autoantibody production, hypergammaglobulinaemia and increased risk for developing B cell non-Hodgkin's lymphoma (Hansen et al., 2007).

Inflammed salivary glands from patients with SjS show a very significant upregulation in BAFF expression, produced in part from T cells (Lavie et al., 2004), which is also found to be elevated in serum, and expected to promote an environment conducive to autoreactive B cell survival. Supporting the importance of this regulator of B cell survival and differentiation in SjS, transgenic mice overexpressing BAFF develop sever sialadenitis and submaxallary gland destruction in a phenotype similar to that of human SjS (Groom et al., 2002).

Peripheral circulating T_{FH} cells are expanded in patients with SjS and also appear in the saliva, the latter correlating with memory B cells and plasma cells suggesting that T_{FH} cells contribute to the pathophysiology of SjS by promoting B cell maturation (Jin et al., 2014). Notably an increase in salivary plasma cell content is positively correlated with serum ANA levels in SjS (Jin et al., 2014). Illustrating the importance of B cell-T cell crosstalk mechanistically in SjS, B cell depletion using rituximab lowers circulating T_{FH} cell levels, IL-17 producing CD4⁺ T cells and serum IL-21 and IL-17, with reductions in circulating T_{FH} cells associating with lower clinical measures of disease activity (Verstappen et al., 2017).

B cell depletion using rituximab has some evidence of effect clinically in SjS, including improvement in salivary gland ultrasound score (Fisher et al., 2018). Supporting a role for enhanced B cell activation in SjS, targeting BAFF using belimumab has efficacy in reducing an index of clinical activity (Mariette et al., 2015).

### Graft-Versus-Host Disease (GVHD)

GVHD is the most frequent life-threatening complication of allogeneic haematopoietic stem cell transplantation. While the immunopathogenesis and initiation of acute GVHD is thought to be driven by immunocompetent T cells in the donated graft tissue recognising the new host as foreign leading to immune activation and attack (Zeiser and Blazar, 2017), there is a significant role for B cells particularly in chronic GVHD.

Underlining defects in B cell homeostasis in GVHD, B cell derived antibodies against histocompatibility antigens (also targets of donor T cells) are evident in GVHD and correlated with disease (Miklos et al., 2005). In both acute and chronic forms of GVHD, dermo-epidermal immunoglobulin deposits in association with C3 complement deposition are observed (Tsoi et al., 1978). Murine models of GVHD have also demonstrated an ability of antibodies from donor B cells to damage the thymus and peripheral lymphoid organs in association with cutaneous pathogenic T_{H}17 infiltration to augment GVHD (Jin et al., 2016).

Patients with chronic GVHD display significantly increased BAFF/B cell ratios compared to patients without GVHD and healthy donors (Sarantopoulos et al., 2009). Notably increased BAFF levels in serum correlate with increases in both circulating pre-germinal centre B cells and plasmablasts (Sarantopoulos et al., 2009). Notably, B cells from patients with chronic GVHD exhibit a heightened metabolic state together with reduced pro-apoptotic signalling priming them for survival (Allen et al., 2012).

Studies in a murine model of chronic GVHD and bronchiolitis obliterans reveal robust germinal centre reactions at the time of disease initiation, organ fibrosis associated with infiltration of B220+ B cells and CD4+ T cells together with alloantibody deposition (Srinivasan et al., 2012). Substantiating the key role of germinal centre formation, the associated follicular T-B cell interaction and pathogenic alloantibody formation, blockade of germinal centre formation suppresses the development of GVHD (Srinivasan et al., 2012). Similarly, depletion of donor splenocyte CD4⁺ T cells in a mouse model of GVHD prevents aberrant germinal centre formation and T_{FH} and germinal centre B cells, while allogeneic splenocytes depleted of B220⁺ B cells also reduced excessive development of both germinal centre B cells and T_{FH} cells, underlining their interdependence (Shao et al., 2015).

B cell depletion using rituximab has proven effective as first line treatment of chronic GVHD, in association with a reduction in circulating ICOS^{hi} PD-1^{hi} T_{FH} cells (Malard et al., 2017).

Thus, in an embodiment, the invention provides (i) a compound selected from clozapine, norclozapine and prodrugs thereof and pharmaceutically acceptable salts and solvates thereof for use in the treatment or prevention of a pathogenic immunoglobulin driven B cell disease with a T cell component in a subject and (ii) a method of treatment or prevention of a pathogenic immunoglobulin driven B cell disease with a T cell component in a subject by administering to said subject an effective amount of a compound selected from clozapine, norclozapine and prodrugs thereof and pharmaceutically acceptable salts and solvates thereof wherein in the case of (i) and (ii) the pathogenic immunoglobulin driven B cell disease with a T cell component is a disease selected from the group consisting of vitiligo, psoriasis, coeliac disease, dermatitis herpetiformis, discoid lupus erythematosus, dermatomyositis, polymyositis, Type 1 diabetes mellitus, autoimmune Addison's disease, multiple sclerosis, interstitial lung disease, Crohn's disease, ulcerative colitis, thyroid autoimmune disease, autoimmune uveitis, primary biliary cirrhosis, primary sclerosing cholangitis, undifferentiated connective tissue disease, autoimmune thrombocytopenic purpura, mixed connective tissue disease, an immune-mediated inflammatory disease (IMID) such as scleroderma, rheumatoid arthritis, Sjogren's disease, an autoimmune connective tissue disease such as systemic lupus erythematosus and graft versus host disease.

In certain diseases, specific Ig types (such as IgG, IgA) are believed to play a role in the pathology of the disease. For example, in dermatitis herpetiformis and coeliac disease, production of pathogenic IgG and IgA are thought to contribute towards the pathology. For example, in multiple sclerosis, vitiligo, autoimmune Addison's disease, type I diabetes mellitus, primary biliary cirrhosis, primary sclerosing cholangitis pathogenic and autoimmune thrombocytopenic purpura, IgG is thought to contribute towards the pathology. The finding by the inventors that clozapine significantly reduces class switched memory B cells and will consequently reduce the numbers of ASCs and the secretion of specific immunoglobulins means that pathogenic IgG levels and pathogenic IgA levels should be reduced. The present inventors have also discovered that clozapine reduces total IgG levels and total IgA levels.

In one embodiment the pathogenic immunoglobulin is pathogenic IgG. In one embodiment the pathogenic immunoglobulin is pathogenic IgA. In one embodiment the pathogenic immunoglobulin is pathogenic IgM.

Preferably, the pathogenic immunoglobulin driven B cell disease with a T cell component is psoriasis, an autoimmune connective tissue disease such as systemic lupus erythematosus, an immune-mediated inflammatory disease (IMID) such as scleroderma, rheumatoid arthritis or Sjogren's disease.

Clozapine is associated with high levels of CNS penetration which could prove to be a valuable property in treating some of these diseases (Michel. L. et al., 2015).

Suitably the compound selected from clozapine, norclozapine and prodrugs thereof inhibits mature B cells, especially CSMBs and plasmablasts, particularly CSMBs. “Inhibit” means reduce the number and/or activity of said cells. Thus, suitably clozapine or norclozapine reduces the number of CSMBs and plasmablasts, particularly CSMBs.

In an embodiment, the compound selected from clozapine, norclozapine and prodrugs thereof has the effect of decreasing CD19 (+) B cells and/or CD19 (−) B-plasma cells.

The term “treatment” means the alleviation of disease or symptoms of disease. The term “prevention” means the prevention of disease or symptoms of disease. Treatment includes treatment alone or in conjunction with other therapies. Treatment embraces treatment leading to improvement of the disease or its symptoms or slowing of the rate of progression of the disease or its symptoms. Treatment includes prevention of relapse.

The term “effective amount” refers to an amount effective, at dosages and for periods of time necessary, to achieve the desired therapeutic result, in which any toxic or detrimental effects of the pharmacological agent are outweighed by the therapeutically beneficial effects. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation. Example dosages are discussed below.

As used herein, a “subject” is any mammal, including but not limited to humans, non-human primates, farm animals such as cattle, sheep, pigs, goats and horses; domestic animals such as cats, dogs, rabbits; laboratory animals such as mice, rats and guinea pigs that exhibit at least one symptom associated with a disease, have been diagnosed with a disease, or are at risk for developing a disease. The term does not denote a particular age or sex. Suitably the subject is a human subject.

It will be appreciated that for use in medicine the salts of clozapine and norclozapine should be pharmaceutically acceptable. Suitable pharmaceutically acceptable salts will be apparent to those skilled in the art. Pharmaceutically acceptable salts include those described by Berge, Bighley and Monkhouse J. Pharm. Sci. (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of clozapine and are included within the scope of this invention.

A compound selected from clozapine, norclozapine and prodrugs thereof and pharmaceutically acceptable salts and solvates thereof may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

A “prodrug”, such as an N-acylated derivative (amide) (e.g. an N-acylated derivative of norclozapine) is a compound which upon administration to the recipient is capable of providing (directly or indirectly) clozapine or an active metabolite or residue thereof. Other such examples of suitable prodrugs include alkylated derivatives of norclozapine other than clozapine itself.

Isotopically-labelled compounds which are identical to clozapine or norclozapine but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”) are also contemplated for the uses and method of the invention. Examples of isotopes that can be incorporated into clozapine or norclozapine include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I, which may be naturally occurring or non-naturally occurring isotopes.

Clozapine or norclozapine and pharmaceutically acceptable salts of clozapine or norclozapine that contain the aforementioned isotopes and/or other isotopes of other atoms are contemplated for use for the uses and method of the present invention. Isotopically labelled clozapine or norclozapine, for example clozapine or norclozapine into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Since clozapine or norclozapine are intended for use in pharmaceutical compositions it will readily be understood that it is preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

In general, clozapine or norclozapine may be made according to the organic synthesis techniques known to those skilled in this field (as described in, for example, U.S. Pat. No. 3,539,573.

A compound selected from clozapine, norclozapine and prodrugs thereof and pharmaceutically acceptable salts and solvates thereof for use in therapy is usually administered as a pharmaceutical composition. Also provided is a pharmaceutical composition comprising clozapine or norclozapine, or a pharmaceutically acceptable salt and/or solvate and/or prodrug thereof and a pharmaceutically acceptable diluent or carrier. Said composition is provided for use in the treatment or prevention of a pathogenic immunoglobulin driven B cell disease with a T cell component in a subject wherein said compound causes mature B cells to be inhibited in said subject.

A compound selected from clozapine, norclozapine and prodrugs thereof and pharmaceutically acceptable salts and solvates thereof may be administered by any convenient method, e.g. by oral, parenteral, buccal, sublingual, nasal, rectal or transdermal administration, and the pharmaceutical compositions adapted accordingly. Other possible routes of administration include intratympanic and intracochlear. Suitably, a compound selected from clozapine, norclozapine and prodrugs thereof and pharmaceutically acceptable salts and solvates thereof are administered orally.

A compound selected from clozapine, norclozapine and prodrugs thereof and pharmaceutically acceptable salts and solvates thereof which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal or pulmonary administration may conveniently be formulated as aerosols, sprays, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal or pulmonary inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatine and glycerine.

Compositions for rectal administration are conveniently in the form of suppositories containing a conventional suppository base such as cocoa butter.

Compositions suitable for topical administration to the skin include ointments, gels and patches.

In one embodiment the composition is in unit dose form such as a tablet, capsule or ampoule.

Compositions may be prepared with an immediate release profile upon administration (i.e. upon ingestion in the case of an oral composition) or with a sustained or delayed release profile upon administration.

For example, a composition intended to provide constant release of clozapine over 24 hours is described in WO2006/059194 the contents of which are herein incorporated in their entirety.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material (i.e. clozapine or norclozapine), depending on the method of administration. The composition may contain from 50 mg to 1000 mg, for example from 100 mg to 400 mg of the carrier, depending on the method of administration. The dose of clozapine or norclozapine used in the treatment or prevention of the aforementioned diseases will vary in the usual way with the seriousness of the diseases, the weight of the sufferer, and other similar factors. However, as a general guide suitable unit doses of clozapine as free base may be 0.05 to 1000 mg, more suitably 1.0 to 500 mg, and such unit doses may be administered more than once a day, for example two or three a day. Such therapy may extend for a number of weeks or months.

A compound selected from clozapine, norclozapine and prodrugs thereof and pharmaceutically acceptable salts and solvates thereof may be administered in combination with another therapeutic agent for the treatment of pathogenic immunoglobulin driven B cell diseases, such as those that inhibit B cells and/or T cells and/or inhibit B cell-T cell interactions. Other therapeutic agents include for example: anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or azathioprine), general anti-inflammatories (such as hydroxychloroquine or NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab). Rituximab may be mentioned in particular.

Other therapies that may be used in combination with the invention include non-pharmacological therapies such as intravenous immunoglobulin therapy (IVIg), subcutaneous immunoglobulin therapy (SCIg) eg facilitated subcutaneous immunoglobulin therapy, plasmapheresis and immunoabsorption.

Thus the invention provides a compound selected from clozapine, norclozapine and prodrugs thereof and pharmaceutically acceptable salts and solvates thereof for use in the treatment or prevention of a pathogenic immunoglobulin driven B cell disease with a T cell component in combination with a second or further therapeutic agent for the treatment or prevention of a pathogenic immunoglobulin driven B cell disease with a T cell component e.g. a substance selected from the group consisting of anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or and azathioprine), general anti-inflammatories (such as hydroxychloroquine and NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab). Rituximab may be mentioned in particular.

When a compound selected from clozapine, norclozapine and prodrugs thereof and pharmaceutically acceptable salts and solvates thereof is used in combination with other therapeutic agents, the compounds may be administered separately, sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations. The individual components of combinations may also be administered separately, through the same or different routes. For example, a compound selected from clozapine, norclozapine and prodrugs thereof and pharmaceutically acceptable salts and solvates thereof and the other therapeutic agent may both be administered orally. Alternatively, a compound selected from clozapine, norclozapine and prodrugs thereof and pharmaceutically acceptable salts and solvates thereof may be administered orally and the other therapeutic agent via may be administered intravenously or subcutaneously.

Typically, a compound selected from clozapine, norclozapine and prodrugs thereof and pharmaceutically acceptable salts and solvates thereof is administered to a human.

### EXAMPLES

### Example 1

### First Observational Study on Human Patients on Anti-Psychotic Therapy

To assess a possible association between antibody deficiency and clozapine use the inventors undertook a cross-sectional case control study to compare the immunoglobulin levels and specific antibody levels (against Haemophilus B (Hib), Tetanus and Pneumococcus) in patients taking either clozapine or alternative antipsychotics.

### Method

Adults (>18 yrs) receiving either clozapine or non-clozapine antipsychotics were recruited during routine clinic visits to ten Community Mental Health Trust (CMHT) outpatient clinics in Cardiff & Vale and Cwm Taf Health Boards by specialist research officers between November 2013 and December 2016 (Table 1). Following consent, participants completed a short lifestyle, drug history and infection questionnaire followed by blood sampling. Where required, drug histories were confirmed with the patient's General Practice records. Formal psychiatric diagnoses and antipsychotic medication use were confirmed using the medical notes, in line with other studies. Patients' admission rates were confirmed by electronic review for the 12-month period prior to recruitment. Patients with known possible causes of hypogammaglobulinemia including prior chemotherapy, carbamazepine, phenytoin, antimalarial agents, captopril, high-dose glucocorticoids, hematological malignancy and 22q11 deletion syndrome were excluded.

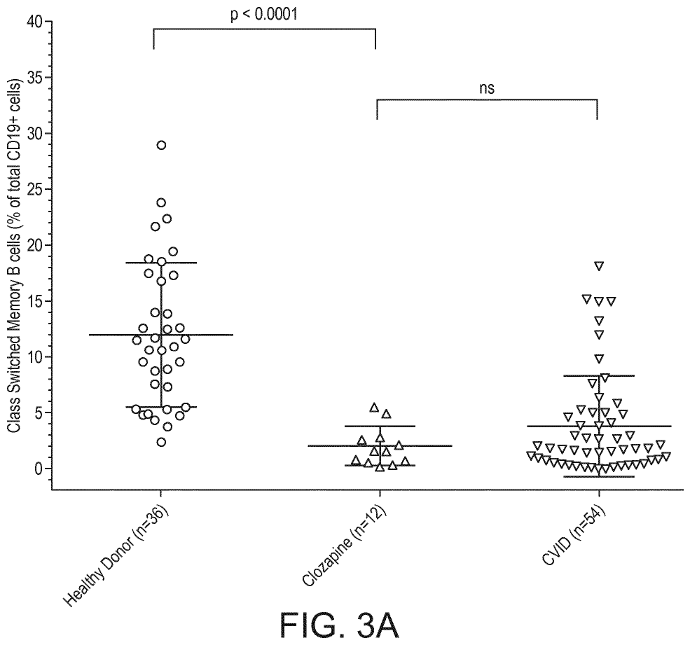

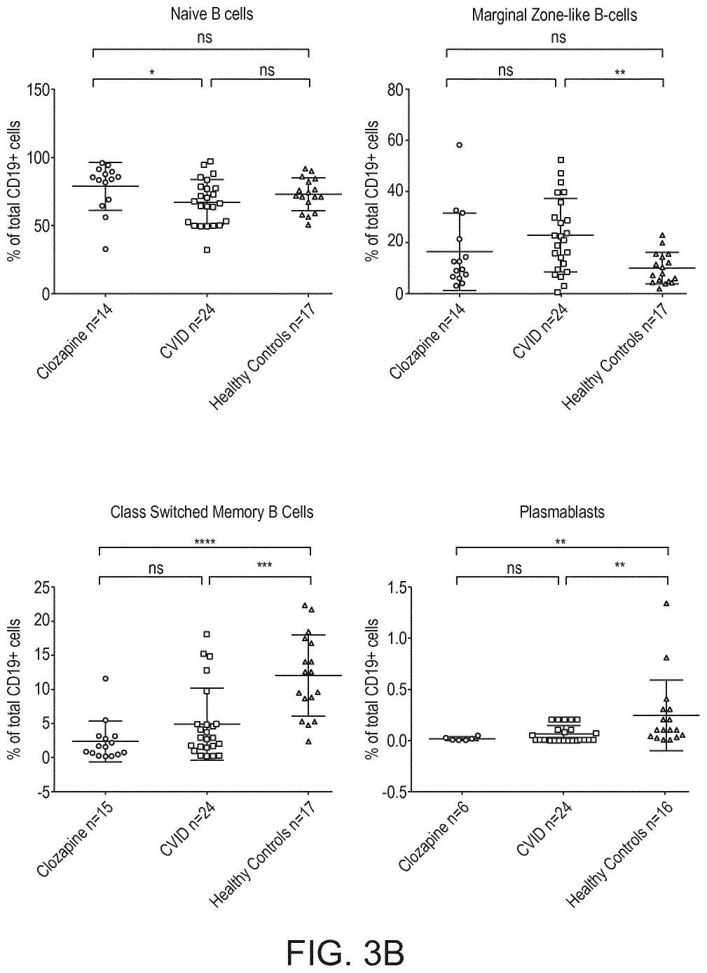

Clinical and immunological data from 13 patients taking clozapine, 11 of whom had been referred independently of the study for assessment in Immunology clinic, are presented in Table 3. Laboratory data on these, healthy controls and patients with common variable immunodeficiency (CVID) are shown in FIG. 3. The 11 independently referred patients were excluded from the overall study analysis.

Immunoglobulin levels (IgG, IgA and IgM) were assayed by nephelometry (Siemens BN2 Nephelometer; Siemens), serum electrophoresis (Sebia Capillarys 2; Sebia, Norcross, Ga., USA) and, where appropriate, serum immunofixation (Sebia Hydrasys; Sebia, Norcross, Ga., USA). Specific antibody titres against Haemophilus influenzae, Tetanus and Pneumococcal capsular polysaccharide were determined by ELISA (The Binding Site, Birmingham, UK). Lymphocyte subsets, naïve T cells and EUROclass B cell phenotyping were enumerated using a Beckman Coulter FC500 (Beckman Coulter, California, USA) flow cytometer. All testing was performed in the United Kingdom Accreditation Service (UKAS) accredited Immunology Laboratory at the University Hospital of Wales. Laboratory adult reference ranges for immunoglobulin levels used were, IgG 6-16 g/L, IgA 0.8-4 g/L, IgM 0.5-2 g/L.

Statistical analysis of the laboratory and clinical data was performed using Microsoft Excel and Graphpad Prism version 6.07 (Graphpad, San Diego, Calif., USA). Independent samples t-test were performed unless D'Agoustino & Pearson testing showed significant deviation from the Gaussian distribution, in which case the non-parametric Mann-Whitney test was used. All tests were two-tailed, using a significance level of p<0.05.

### Results

### Study Participants

A total of 291 patients taking clozapine and 280 clozapine-naïve patients were approached and 123 clozapine and 111 clozapine-naïve patients consented to the study (Table 1). Recruitment was stopped as per protocol when the target of 100 patients in each group had been achieved. There were small differences in gender with more males in the clozapine-treated group (53% versus 50%) and a lower mean age in the clozapine group (45 versus 50 years). These differences are unlikely to be relevant as there are no gender differences in the adult reference range for serum immunoglobulins and there is a male predominance in schizophrenia. Levels of smoking, diabetes, COPD/asthma, and alcohol intake were similar between the groups. More patients were admitted to hospital with infection in the clozapine group (0.12 vs 0.06 per patient year) and more took >5 courses of antibiotics per year compared with controls (5.3% vs 2%). The possible impact of a diagnosis of schizophrenia, medications and smoking as risk factors for antibody deficiency were assessed in a subgroup analysis (Table 2).

TABLE 1
Clozapine-treated and clozapine-naïve patient characteristics

| | Clozapine- | Clozapine- |
|---|---|---|
| | Treated | Naive |
| Total screened | 291 | 280 |
| Declined, lacked capacity, or unable to | 168 | 169 |
| obtain blood sample | | |
| Initial Screening | 123 | 111 |
| Sex (M:F) | (81:42) | (56:55) |
| Mean age, years | 45.3 | 50.3 |
| (Range) | (22.0-78.0) | (21.6-78.0) |
| Post-exclusion | 94 | 98 |
| (% total screened) | (32%) | (35%) |
| Sex (M:F) | 64:30 | 54:44 |
| Mean age, years | 44.4 | 50.4 |
| (Range) | (22.0-78.0) | (21.6-78.0) |
| Primary Psychiatric Diagnosis | | |
| Schizophrenia | 87 | 58 |
| Schizoaffective | 1 | 5 |
| Bipolar | 0 | 11 |
| Psychosis | 0 | 15 |
| Depression | 0 | 3 |
| Personality Disorder | 2 | 2 |
| Anxiety disorder | 0 | 2 |
| Electronic record incomplete | 4 | 2 |
| Dual antipsychotic treatment | 30.9% | 11.2% |
| Duration antipsychotic use | 8.0 | 7.0 |
| (median, range), years | (0.1-20) | (0.1-44) |
| Current smoking (%) | 60.6% | 56.1% |
| Diabetes (%) | 20.2% | 17.3% |
| COPD/Asthma (%) | 13.8% | 16.3% |
| Alcohol intake mean (units/week), | 5.3 (0- | 6.0 (0- |
| range | 60) | 68) |
| Antibiotic courses per year | | |
| Nil courses | 61.7% | 63.3% |
| 1-5 courses | 33.0% | 34.7% |
| >5 courses | 5.3% | 2.0% |
| Admission frequency in | | |
| 12-month period | | |
| All cause | 21 (14 | 14 (13 |
| | patients) | patients) |
| Infection-related | 15 (10 | 7 (6 |
| | patients) | patients) |

### Effects of Clozapine on Antibody Levels

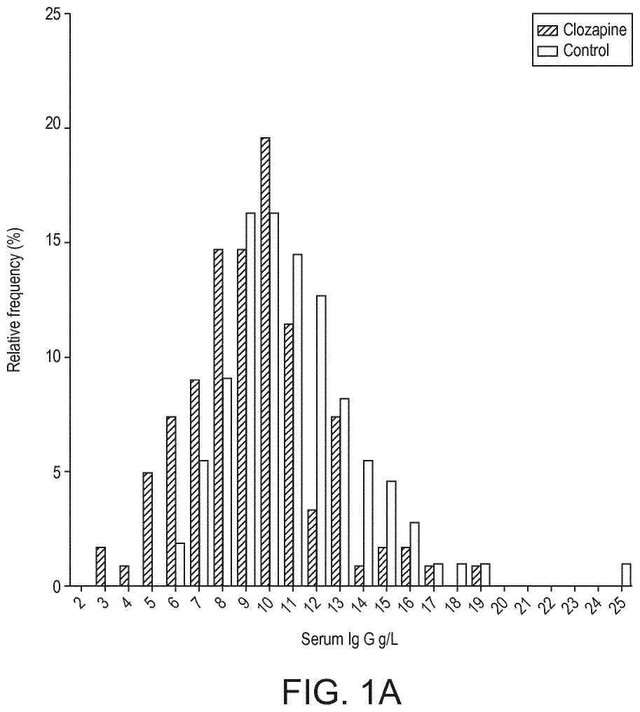

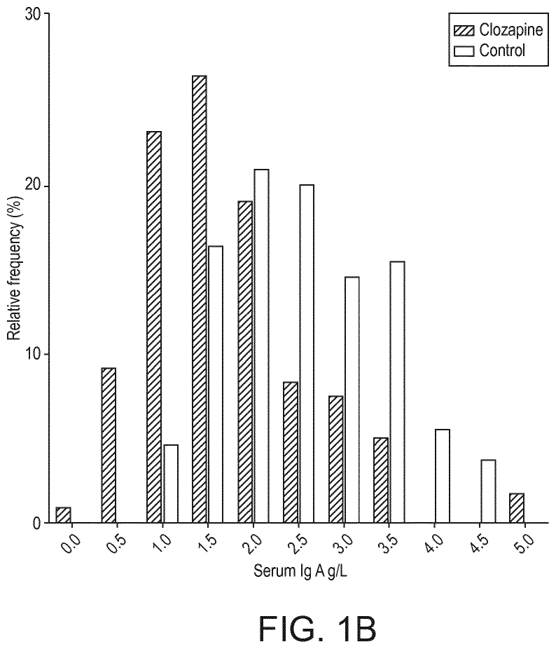

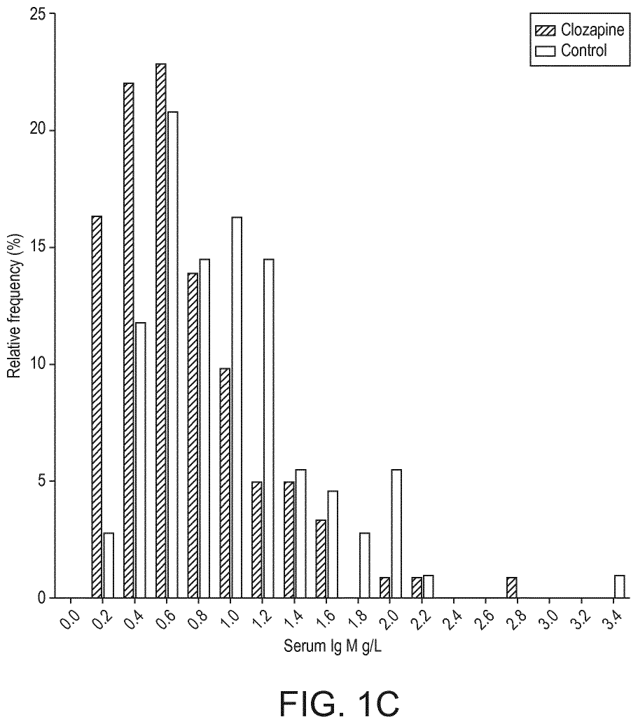

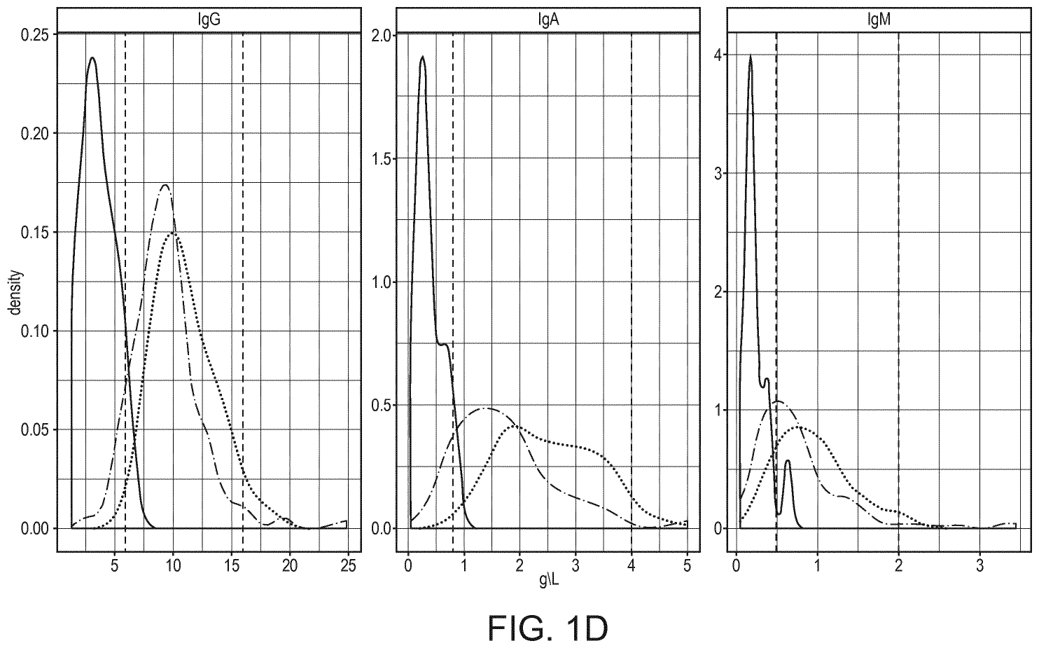

FIG. 1A-C shows significantly reduced concentrations of all three immunoglobulin classes (IgG, IgA and IgM) in patients receiving clozapine, with a shift towards lower immunoglobulin levels in the distribution as a whole for each of IgG, IgA and IgM compared to the clozapine-naïve control group. The percentages of the 123 patients having immunoglobulin levels below the reference range were IgG 9.8% (p<0.0001), IgA 13.0% (p<0.0001) and IgM 38.2% (p<0.0001) compared with the 111 clozapine-naïve IgG 1.8%, IgA 0.0% and IgM 14.4%. Large percentages of both clozapine-treated and clozapine-naïve patients had specific antibody levels below the protective levels for HiB (51% and 56% less than 1 mcg/ml, (Orange et al., 2012)), Pneumococcus (54% and 56% less than 50 mg/L, (Chua et al., 2011)) and Tetanus (12% and 14% less than 0.1 IU/ml). The Pneumococcal IgA (31 U/ml vs 58.4 U/ml p<0.001) and IgM (58.5 U/ml vs 85.0 U/ml p<0.001) levels are significantly lower in clozapine-treated versus clozapine-naïve patients.

Subgroup analysis (Table 2) was undertaken to determine if the reductions in immunoglobulins were potentially explained by confounding factors including any other drugs, a diagnosis of schizophrenia and smoking. The assessment of the effect of excluding other secondary causes of antibody deficiency (plus small numbers where additional diagnoses were uncovered—Table 1) is shown in Column B. The number of patients excluded on the basis of taking anti-epileptic medications was higher in the clozapine-treated group and is likely to reflect the use of these agents for their mood stabilizing properties rather than as treatment for epilepsy.

TABLE 2
Immunoglobulin levels and specific antibody levels in sub-groups A-D

| | A | | B | | C | | D | |
|---|---|---|---|---|---|---|---|---|
| Medication: | Clozapine | Control | Clozapine | Control | Clozapine | Control | Clozapine | Control |
| Diagnosis: | All | All | All | All | Schizophrenia | | All | All |
| | | | | | diagnoses only | | | |
| Smoking: | All | All | All | All | All | All | Smokers only | |
| Possible | No | No | Yes | | Yes | | Yes | |
| secondary | | | | | | | | |
| causes | | | | | | | | |
| excluded | | | | | | | | |
| Sample size: | 123 | 111 | 94 | 98 | 87 | 58 | 57 | 55 |
| Serum IgG | 95% CI: | | 95% CI: 0.98 | | 95% CI: 0.92 | | Non-Gaussian | |
| (Reference | 0.89-2.32 **** | | to 2.59 **** | | to 2.77 *** | | distribution ^{†} | |
| range 6-16 g/L) | | | | | | | | |
| <3 | 0.8% | 0.0% | 1.1% | 0.0% | 1.2% | 0.0% | 1.8% | 0.0% |
| <4 | 1.6% | 0.0% | 1.1% | 0.0% | 1.2% | 0.0% | 1.8% | 0.0% |
| <5 | 3.3% | 0.0% | 2.1% | 0.0% | 2.3% | 0.0% | 1.8% | 0.0% |
| <6 | 9.8% | 1.8% | 8.4% | 1.0% | 9.2% | 1.7% | 8.8% | 1.8% |
| Serum IgA | 95% CI: 0.55 | | 95% CI: 0.55 | | 95% CI: 0.59 | | 95% CI: 0.41 | |
| (Reference | to 1.01 **** | | to 1.05 **** | | to 1.19 **** | | to 1.04 **** | |
| range 0.8-4.0 g/L) | | | | | | | | |
| <0.5 | 1.6% | 0.0% | 2.1% | 0.0% | 2.3% | 0.0% | 3.5% | 0.0% |
| <0.6 | 2.4% | 0.0% | 2.1% | 0.0% | 3.5% | 00% | 3.5% | 0.0% |
| <0.7 | 6.5% | 0.0% | 6.4% | 0.0% | 6.9% | 0.0% | 3.5% | 0.0% |
| <0.8 | 13.0% | 0.0% | 13.8% | 0.0% | 14.9% | 0.0% | 10.5% | 0.0% |
| Serum IgM | Non-Gaussian | | 95% CI: 0.10 | | 95% CI: 0.06 | | 95% CI: 0.02 | |
| (Reference | distribution ^{††††} | | to 0.38 *** | | to 0.38 ** | | to 0.39 * | |
| range 0.5-1.9 g/L) | | | | | | | | |
| <0.2 | 8.1% | 0.0% | 5.3% | 0.0% | 5.8% | 0.0% | 1.78% | 0.0% |
| <0.3 | 16.3% | 2.7% | 12.8% | 3.1% | 12.6% | 5.2% | 12.3% | 1.8% |
| <0.4 | 29.3% | 8.1% | 26.6% | 8.2% | 27.6% | 6.9% | 26.3% | 9.1% |
| <0.5 | 38.2% | 14.4% | 34.0% | 15.3% | 35.6% | 13.8% | 33.3% | 18.2% |
| IgG- | 95% CI: −8.25 | | 95% CI: −11.21 | | 95% CI: −20.50 | | 95% CI: −23.64 | |
| Pneumococcus | to 21.92 (ns) | | to 22.63 (ns) | | to 17.54 (ns) | | to 21.70 (ns) | |
| (mg/L) | | | | | | | | |
| <35 | 39.0% | 43.2% | 38.3% | 40.8% | 37.9% | 43.1% | 45.6% | 43.6% |
| <50 | 53.7% | 55.9% | 52.1% | 54.1% | 50.6% | 60.3% | 54.4% | 63.6% |
| IgG- Tetanus | Non-Gaussian | | Non-Gaussian | | Non-Gaussian | | Non-Gaussian | |
| (IU/ml) | distribution (ns) | | distribution (ns) | | distribution (ns) | | distribution (ns) | |
| <0.1 | 12.2% | 13.5% | 10.6% | 13.3% | 11.5% | 13.8% | 12.3% | 14.6% |
| IgG- | Non-Gaussian | | Non-Gaussian | | Non-Gaussian | | Non-Gaussian | |
| Haemophilus | distribution (ns) | | distribution (ns) | | distribution (ns) | | distribution (ns) | |
| B (mcg/ml) | | | | | | | | |
| <1.0 | 51.2% | 55.9% | 51.1% | 54.1% | 49.4% | 53.5% | 50.9% | 60.0% |
| Sample size: | 118 | 85 | 89 | 77 | 84 | 45 | 54 | 45 |
| IgA- | 31 ± | 58.4 ± | 30.8 ± | 58.8 ± | 31.6 ± | 49.9 ± | 30.7 ± | 61.3 ± |
| Pneumococcus (U/) | 3.97 *** | 6.7 | 4.7 ^{††††} | 7.0 | 4.9 ^{†††} | 7.6 | 52 ^{††††} | 25 |
| IgM- | 58.5 ± | 85 ± | 59.8 ± | 85.8 ± | 60.4 ± | 78.6 ± | 61.6 ± | 91.7 ± |
| Pneumococcus (U/L) | 42 *** | 62 | 4.9 ** | 7.4 | 5.1 * | 7.1 | 7.0 ^{††} | 10.3 |

Data shown as mean ± 1 SEM unless otherwise stated.
* Independent T test (normally distributed) or
† Mann-Whitney (non-normally distributed)
Levels of significance:
*/† p < 0.05,
**/†† p < 0.005,
***/††† p < 0.0005,
****/†††† p < 0.0001

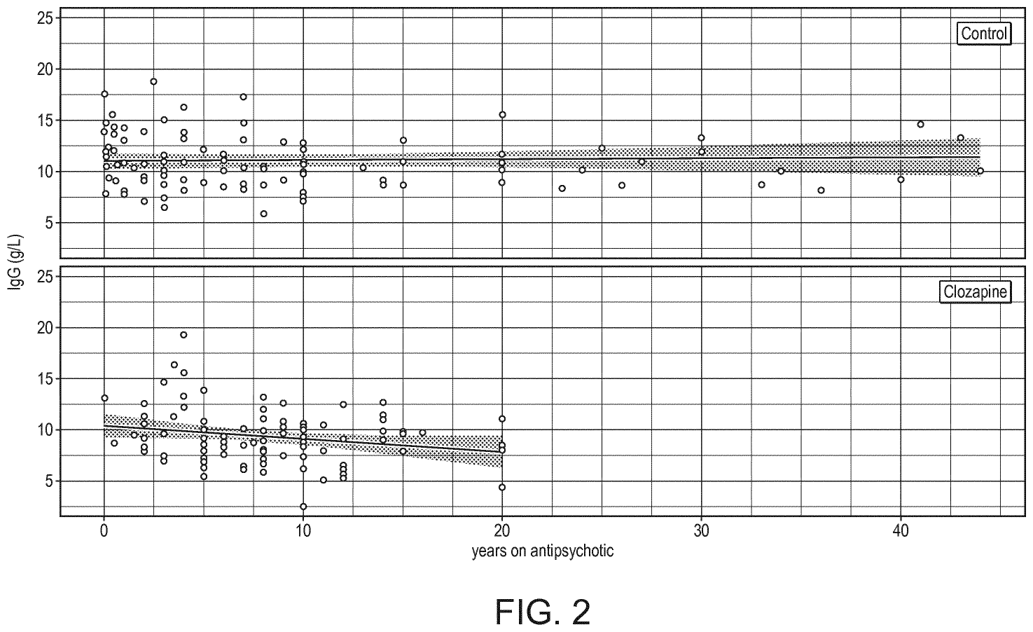

The association of clozapine with reduced IgG, IgA, IgM and Pneumococcal IgA and IgM remained statistically significant in all subgroups with 95% confidence intervals including when psychiatric diagnoses were restricted to schizophrenia only (Column C), and when non-smokers were excluded (Column D). When secondary causes of antibody deficiency were excluded (Column B) the odds ratios (with 95% confidence interval) for reduced immunoglobulins were IgG 9.02 (1.11-73.7), IgA: 32.6 (1.91-558) and IgM: 2.86 (1.42-5.73). In addition, a longer duration of clozapine therapy is associated with lower serum IgG levels (p 0.014) shown in FIG. 2. This is not observed in clozapine-naïve patients treated with alternative antipsychotic drugs, despite a longer treatment duration than the clozapine therapy group.

### Immunological Assessment of Referred Patients Taking Clozapine

Thirteen patients on clozapine were independently referred for assessment of antibody deficiency to Immunology clinic. Two had previously been recruited to the study and the eleven others are not included in the study to avoid bias. Five of the thirteen patients had been identified through the all Wales calculated globulin screening program. It was thus possible to undertake a more detailed immunological assessment in this group of thirteen ‘real life’ patients to provide additional background information (Table 3).

TABLE 3
Immunological characteristics of the 13 referred clozapine patients

| Referral | | | Relevant | Clozapine | CSMB | | | Follow-up/ |
|---|---|---|---|---|---|---|---|---|
| Reason | Age | Smoking | Medication | duration | (6.5-29.1%) | | Intervention | months |
| Recurrent | 47 | 20 | Clozapine | >4 | IgG < 1.34 | 0.3% | Prophylactic antibiotics | 120 |
| respiratory | | pack | 250 mg | | | | Failure to respond to | |
| tract | | years | Sodium | | | | haemophilus and | |
| infection | | | Valproate | | IgA < 0.22 | | pneumococcal | |
| (12 per | | | 1 g | | IgM < 0.17 | | vaccination. | |
| year). | | | Risperidone | | | | Commenced SCIg 9.6 g | |
| | | | | | | | weekly in nursing home. | |
| | | | | | | | Recently discontinued | |
| | | | | | | | clozapine due to | |
| | | | | | | | neutropenia. | |
| Low | 46 | 42 | Clozapine | 15 | IgG 5.24 | 2.77% | Prompt antibiotic | 69 |
| calculated | | pack | 575 mg | | IgA 0.49 | | therapy | |
| globulin | | years | Senna, | | IgM 0.41 | | Durable pneumococcal | |
| Included in | | | fibrogel, | | | | vaccine response | |
| study | | | cyclizine | | | | Continues clozapine | |
| Low | 51 | 34 | Clozapine | 5 | IgG 2.68 | 5.50% | Prophylactic antibiotics | 48 |
| calculated | | pack | 200 mg | | IgA 0.38 | | Failure to responds to | |
| globulin. | | years | Amisulpride | | IgM < 0.17 | | haemophilus and | |
| | | | | | | | pneumococcal | |
| | | | | | | | vaccination. | |
| | | | | | | | Continues clozapine, | |
| | | | | | | | Considering | |
| | | | | | | | immunoglobulin | |
| | | | | | | | replacement | |
| Persistent | 63 | 60 | Clozapine | 7.5 | IgG 2.98 | 0.5% | Prophylactic antibiotics | 42 |
| cough for | | pack | 400 mg | | IgA < 0.22 | | Non-durable | |
| over a year | | years | Olanzapine | | IgM 0.23 | | pneumococcal vaccine | |
| and | | | Trihexyphenidyl | | | | response | |
| remains | | | | | | | Commenced IVIg 40 g 3 | |
| productive | | | | | | | weekly | |
| of green | | | | | | | Clozapine stopped with | |
| sputum | | | | | | | resultant psychotic | |
| despite | | | | | | | episode. | |
| several | | | | | | | Clozapine restarted | |
| courses of | | | | | | | with GCSF cover | |
| antibiotics. | | | | | | | Continues on SCIg and | |
| | | | | | | | clozapine. | |
| Recurrent | 49 | 55 | Clozapine | 7 | IgG 1.2 | 0.14% | Prophylactic antibiotics | 32 |
| respiratory | | pack | 300 mg | | IgA | | Failure to respond to | |
| infections | | years | Sodium | | undetectable | | pneumococcal | |
| Low | | | Valproate, | | IgM 0.07 | | vaccination. | |
| calculated | | | Pirenzapine, | | | | IVIg 40 g 3 weekly | |
| globulins | | | aripiprazole | | | | Continues clozapine | |
| Recurrent | 63 | 20 | Clozapine | 10 years | IgG 3.3 | 1.58% | Prophylactic | 24 |
| chest | | pack | 250 mg - | Stopped | IgA 0.26 | | azithromycin: 4 chest | |
| infections | | years, | stopped | 24 | IgM 0.41 | | infections in 3 months | |
| Low | | stopped | Lithium | months | | | Failure to respond to | |
| calculated | | 30 | 400 mg | ago | | | pneumococcal | |
| globulin | | years | Levothyroxine | | | | vaccination | |
| | | ago | Calchichew | | | | Clozapine stopped- red | |
| | | | Citalopram | | | | flags with neutropenia | |
| | | | | | | | IgG rose to 5.95 from | |
| | | | | | | | 3.3 g/L, IgA 0.29, IgM | |
| | | | | | | | 0.49 after 24 months | |
| | | | | | | | CSM Brose to 2.77% | |
| 7 courses of | 59 | 47 | Clozapine | 10 | IgG 2.38 | 2.54% | Prophylactic antibiotics | 15 |
| antibiotics | | pack | 450 mg | | IgA < 0.22 | | Failure to respond to | |
| for chest | | years | Omeprazole, | | IgM < 0.17 | | pneumococcal | |
| infections | | | pirenzapine, | | | | vaccination. | |
| past 12 | | | venlafaxine, | | | | Commenced IVIg 30 g 3- | |
| months, 9 | | | metformin, | | | | weekly | |
| GP visits | | | saxagliptin, | | | | Continues clozapine | |
| No | | | atorvastatin | | | | | |
| clozapine | | | | | | | | |
| red-flags | | | | | | | | |
| Included in | | | | | | | | |
| study | | | | | | | | |
| Recurrent | 46 | 74 | Clozapine | 21 | IgG 4.24 | 0.84% | Prophylactic antibiotics | 12 |
| respiratory | | pack | 450 mg | | IgA < 0.22 | | Failure to respond to | |
| infections | | years | Sertaline, | | IgM < 0.17 | | pneumococcal | |
| | | | montelukast, | | | | vaccination. | |
| | | | simvastatin, | | | | Commenced SCIg | |
| | | | seretide, | | | | Continues clozapine | |
| | | | salbulatamol, | | | | | |
| | | | temazepam | | | | | |
| Recurrent | 50 | 60 | Clozapine | >7 | IgG 6.65 | 4.95% | Prophylactic antibiotics | 12 |
| respiratory | | pack | 700 mg | | IgA < 0.22 | | Failure to responds to | |
| tract | | years | Amisulpride, | | IgM < 0.17 | | haemophilus and | |
| infections | | | cholecalciferol, | | | | pneumococcal | |
| | | | cod | | | | vaccination. | |
| | | | liver oil | | | | Continues clozapine | |
| Low | 51 | 12 | Clozapine | 11 | IgG 5.61 | 2.10% | Prompt antibiotic | 6 |
| calculated | | pack | 575 mg | | IgA 0.81 | | therapy | |
| globulin | | years | Fibrogel, | | IgM 0.18 | | Failure to respond to | |
| | | | lactulose, | | | | pneumococcal | |
| | | | cod liver | | | | vaccination. | |
| | | | oil, | | | | Continues clozapine | |
| | | | citalopram | | | | | |
| Recurrent | 61 | 15/day | Clozapine | >4 | IgG 4.79 | 1.49% | Prompt antibiotics | 6 |
| skin | | | 325 mg | | IgA 0.63 | | Assessment of vaccine | |
| infections | | | Sodium | | IgM < 0.17 | | responses ongoing | |
| | | | valproate, | | | | Continues clozapine | |
| | | | metformin, | | | | | |
| | | | exenatide, | | | | | |
| | | | ciitalopram, | | | | | |
| | | | Fultium | | | | | |
| | | | D3, | | | | | |
| | | | Omeprazole, | | | | | |
| Calculated | 36 | 35 | Clozapine - | Stopped | IgG 4.8 | N/A | Declined further blood | 5 |
| globulin | | pack | stopped 2 | | IgA 0.54 | | tests | |
| | | years | years prior | | IgM 0.3 | | | |
| | | | to referral | | | | | |
| | | | Procyclidine, | | | | | |
| | | | folic acid, | | | | | |
| | | | diazepam, | | | | | |
| | | | paracetamol | | | | | |
| Recurrent | 57 | 20-40 | Clozapine | >4 | IgG < 1.34 | 0.3-0.7% | Prophylactic antibiotics | 42 |
| respiratory | | pack | 750 mg | | IgA < 0.22 | | Failure to respond to | |
| tract | | years | Amisulpride | | IgM < 0.17 | | pneumococcal | |
| infections. | | | | | | | vaccination | |
| Clozapine- | | | | | | | IVIg 40 g every 3 weekly | |
| induced | | | | | | | Stopped clozapine | |
| sialorrhoea. | | | | | | | during chemotherapy | |

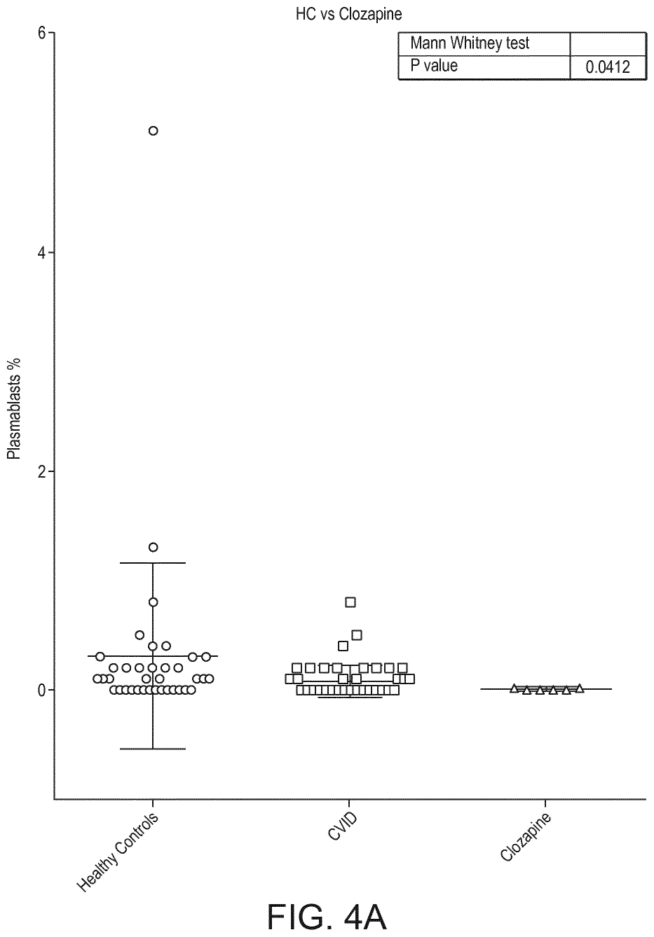

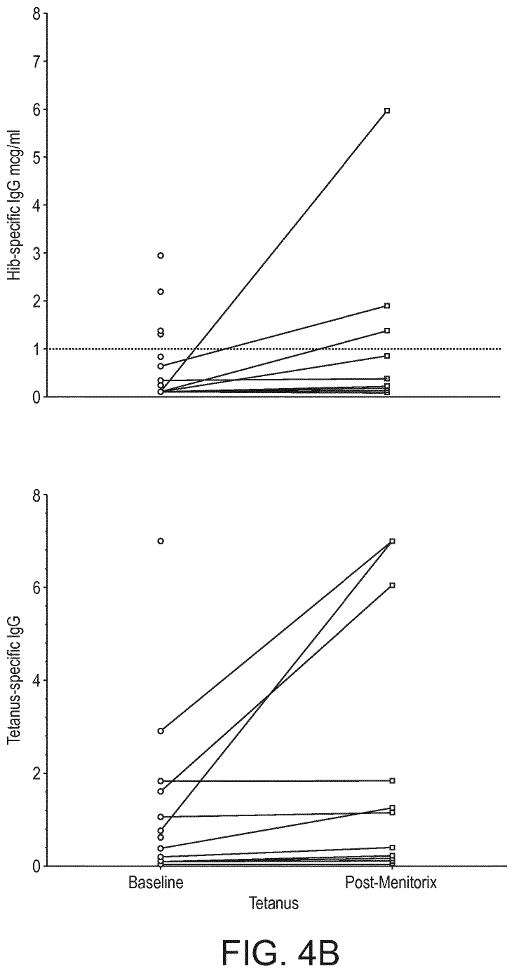

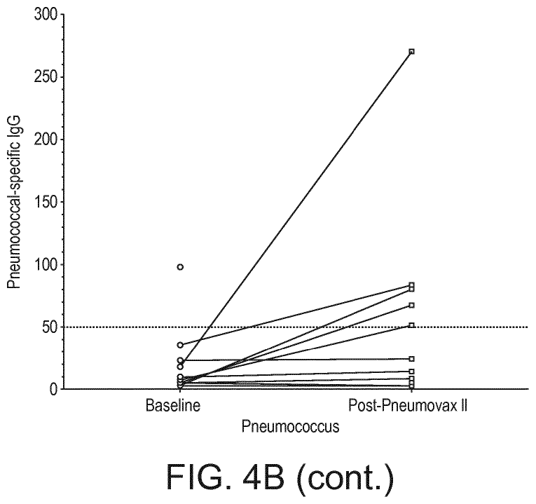

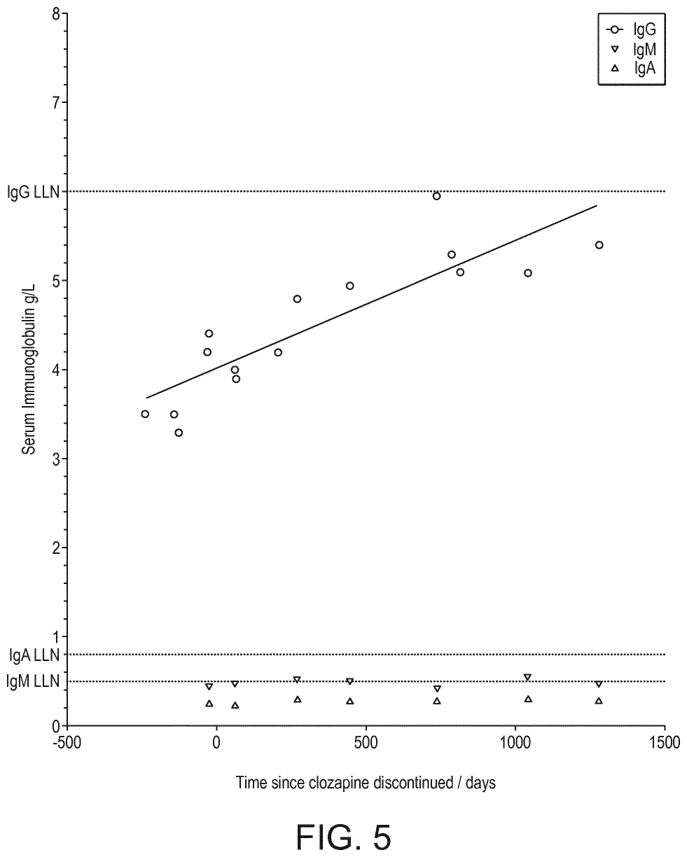

Certain additional analysis shown in FIGS. 1D, 3B, 4B and 5 was done on a slightly different set of referred clozapine patients comprising the 13 referred to in Table 3, plus 4 additionally recruited patients. In respect of FIG. 1D, 4 of the 17 patients were removed for various reasons therefore the number of patients for which data is presented is 13. In respect of FIG. 3B, the number of patients for which data is presented is shown in the Figure. In respect of FIG. 4B, the number of patients for which data is presented is stated below. In respect of FIG. 5, the number of patients for which data is presented is 15.

Immunoglobulins were reduced in all patients (mean IgG 3.6 g/L, IgA 0.34 g/L and IgM 0.21 g/L). There was no severe overall lymphopenia or B cell lymphopenia, however, all patients had a major reduction in the percentage of CSMB (mean 1.87%, reference range 6.5-29.1%). A substantial reduction of CSMB is characteristic of patients with common variable immunodeficiency (CVID), the commonest severe primary immunodeficiency in adults. The percentages of CSMB in these clozapine-treated and CVID patients compared to healthy controls are shown in FIG. 3A (p<0.0001), The plasmablast levels for 6 of the clozapine patients compared to CVID patients and healthy controls are shown in FIG. 4A (p=0.04) and in FIG. 3B with age matched CVID and healthy controls. A reduction of plasmablasts is also characteristic of patients with common variable immunodeficiency (CVID) and this was also observed in clozapine treated patients. Responses to vaccination were impaired in 10/11 patients assessed and management included emergency backup antibiotics for 2/13 patients, prophylactic antibiotics in 9/13 and 6/13 patients were treated with immunoglobulin replacement therapy (IGRT). No patients discontinued clozapine because of antibody deficiency. The inflammatory or granulomatous complications which occur in a subset of CVID patients were not observed.

Vaccine specific-IgG responses are routinely evaluated as part of clinical assessment and summarised in FIG. 4B. At initial assessment, levels below putative protective threshold were common with IgG to Haemophilus influenza B (HiB) <1 mcg/ml in 12/16 patients (75%); Pneumococcus-IgG <50 mg/L in 15/16 patients (94%); and Tetanus-IgG <0.1 IU/mL in 6/16 patients (38%) individuals tested. Post-Menitorix (HiB/MenC) vaccination serology was assessed after 4 weeks, with 5/12 (42%) individuals failing to mount a Haemophilus-IgG response ≥1 mcg/ml, and 1/12 failing to exceed the ≥0.1 IU/mL post-vaccination Tetanus-IgG level defined by the World Health Organisation. Following Pneumovax II, 8/11 (73%) individuals failed to develop an IgG response above a threshold of ≥50 mg/L.

FIG. 5 shows a gradual recovery in terms of the serum IgG level from 3.5 g/L to 5.95 g/L over 3 years but without clear improvement in IgA or IgM following cessation of clozapine.

One patient subsequently discontinued clozapine because of neutropenia which normalized on clozapine cessation. Over the following 24 months the serum IgG level gradually increased from 3.3 g/L to 4.8 g/L and then 5.95 g/L while IgA and IgM remained low. The increase in IgG was accompanied by a concomitant increase in class switched memory B cells from 1.58-2.77%, suggesting a gradual recovery on withdrawal of clozapine.

FIG. 1D shows a density plot showing distribution of serum immunoglobulin levels in patients receiving clozapine referred for Immunology assessment. Serum immunoglobulin distributions for clozapine-treated (n=94) and clozapine-naive (n=98) are also shown for comparison—adapted from (Ponsford et al., 2018b). Dotted lines represent the 5^{th} and 95^{th} percentiles for healthy adults. A leftward shift (reduction) in the distribution curves of total immunoglobulin is observed in patients on clozapine for each of IgG, IgA and IgM compared to clozapine naive patients; this finding was particularly marked for the additionally recruited clozapine referred patients.

### Summary of Results

Clozapine treatment in patients led to a significant reduction of all immunoglobulin types. Percentages of patients below the immunoglobulin reference ranges were higher in clozapine treated (n=123) as compared with clozapine naive patients (n=111) (IgG <6 g/L: 9.8% vs 1.8%; IgA <0.8 g/L: 13.1% vs 0..0%; IgM <0.5 g/L: 38.2% vs 14.2%) (p<0.0001) (see FIG. 1A-C)

Extending the duration of clozapine treatment was associated with progressively reduced IgG levels in patients treated with clozapine but not in clozapine naive patients who were on other antipsychotic medication (see FIG. 2).

Notably the effect of clozapine on IgG levels was seen to be reversible, albeit slowly (years), consistent with an impact of clozapine on long-lived IgG+ plasma cells in particular.

Specific IgG antibodies were below protective levels in both clozapine-treated and clozapine-naïve groups (HiB 51.2% vs 55.9%; Pneumococcal 53.7% vs 55.9%; Tetanus 12.2% vs 13.5%)). However, pneumococcal IgA and IgM levels were significantly lower in clozapine-treated patients as compared with clozapine-naïve patients (IgA 31.0 U/L vs 58.4 U/L; IgM 58.5 U/L vs 85 U/L) (p<0.001) (see Table 2).

Mean levels of CSMBs were significantly reduced at 1.87% in clozapine-treated patients referred independently to clinic and not included in the overall study (n=12) and in CVID patients (n=54) as compared with healthy controls (n=36) and the reference range of 6.5-29.1% (p<0.0001) (see FIG. 3A). Mean levels of plasmablasts were also reduced in clozapine-treated patients (p=0.04).

FIG. 3B shows an extension of the data in FIG. 3A in which referred clozapine patients are compared to age matched CVID and health control subjects. The first graph shows that total B cell numbers are similar between clozapine, CVID and healthy controls and the second graph demonstrates no significant difference between clozapine treated and healthy control marginal zone B cell numbers while there is an increased number observed in CVID patients. The lower two graphs show a significant reduction in both CSMB and plasmablasts in both clozapine treated and CVID patients over healthy controls.

### Example 2

### Second Observational Study on Human Patients on Anti-Psychotic Therapy

Using a cross-sectional observational design in patients on anti-psychotic therapy, this study seeks to test the association between clozapine use, immunophenotype—specifically circulating B cell subsets and immunoglobulin levels—and documented infections, in comparison to other anti-psychotic medication. The study is recruiting patients established on clozapine and those on other antipsychotic drugs from Ashworth Hospital and outpatients from community mental health services in Mersey Care NHS Foundation Trust. The findings will partly provide validation of those from the initial observational study in an orthogonal population, in addition to extending insights into the impact of clozapine on B cell populations through more detailed immunophenotypic analysis.

The study entails a single blood test for detailed immunological analysis and completion of a clinical research form-based questionnaire detailing important clinical parameters including documented infection history, past medical history and concurrent medication use. The findings will be analysed to identify any association between clozapine, circulating B cell levels/function and immunoglobulin levels, its frequency and severity, as well as specificity in relation to other antipsychotic medications.

### Study Aims and Objectives

The specific research questions this study seeks to answer are:

### Primary Outcomes:

i) Is chronic treatment with clozapine associated with (a) a higher proportion of those with specific B cell subsets (namely class-switched memory B cells and plasma cells) below reference ranges and (b) a higher proportion of those with circulating immunoglobulin levels (IgG, IgA and IgM) below references compared to proportions below reference range observed in controls?

### Secondary Outcomes:

ii) Is clozapine associated with reductions in specific antibodies (e.g. pneumococcus, tetanus and Hib) compared to controls?
iii) Is clozapine use associated with an effect on circulating T cells (number/function) compared to controls?
iv) Is clozapine associated with a higher frequency of infections and antibiotic use than controls?
v) Are the primary outcomes related to duration of clozapine therapy?

### Immune Biomarkers

The following immune biomarkers are tested:
1. Total IgG IgM, IgA, and serum electrophoresis with immunofixation if appropriate;
2. Specific IgG levels—tetanus toxoid, pneumococcus, Hib (±IgA and IgM for pneumococcus);
3. Detailed immune cell phenotyping through FACS analysis, including:
   a. Lymphocyte phenotypes—(including CD3, CD4, CD8, CD19, CD56)
   b. B cell panel (based on the EUROClass classification of B cell phenotype (Wehr et al., 2008)) which includes CSMB cells and plasmablasts
   c. Naïve T cell panel
4. RNA extraction from PBMCs (whole blood stored in a RNA preservation solution, e.g. Universal container with ^{˜}4-5 mL RNALater or in PAXgene tube to preserve RNA integrity) for subsequent RNA transcription analysis

All immune biomarker samples are processed and analysed in a UKAS Accredited validated NHS laboratory.

### Results

At the time of writing this study is still recruiting but an interim analysis of the available collected immunophenotypic data (approximately 2/3rds of the way through recruitment) has been undertaken with the caveat that this represent a proportion of the final projected sample size (n 100).

The major findings so far are detailed below:

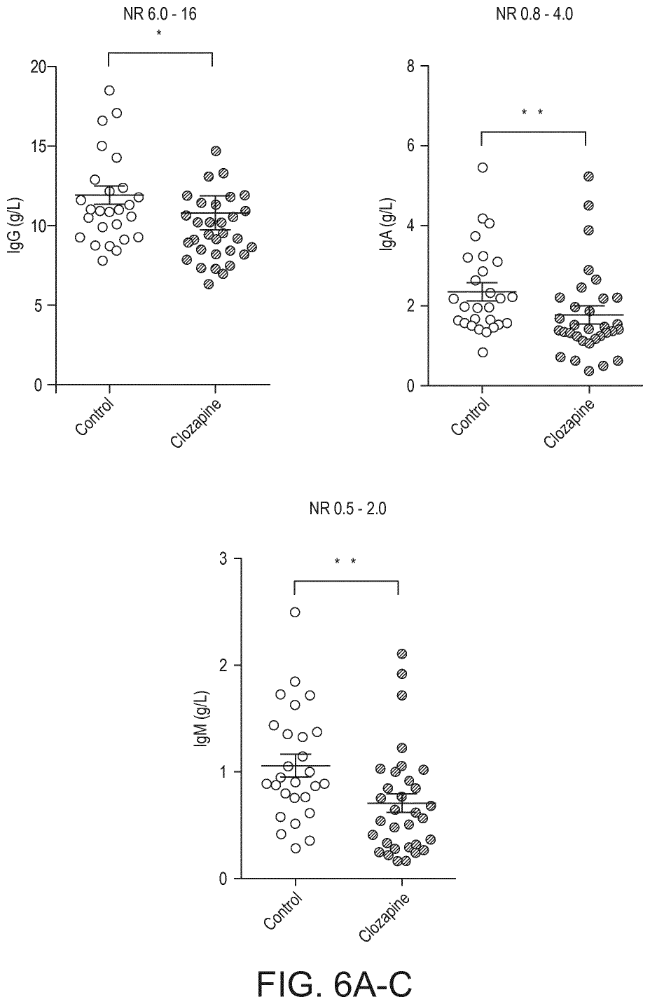

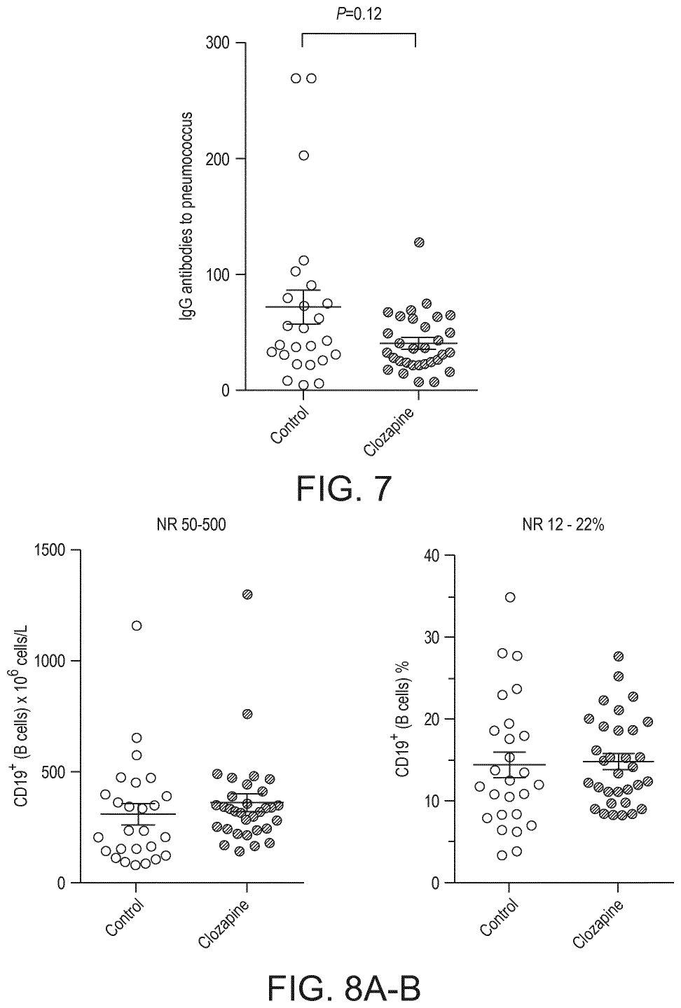

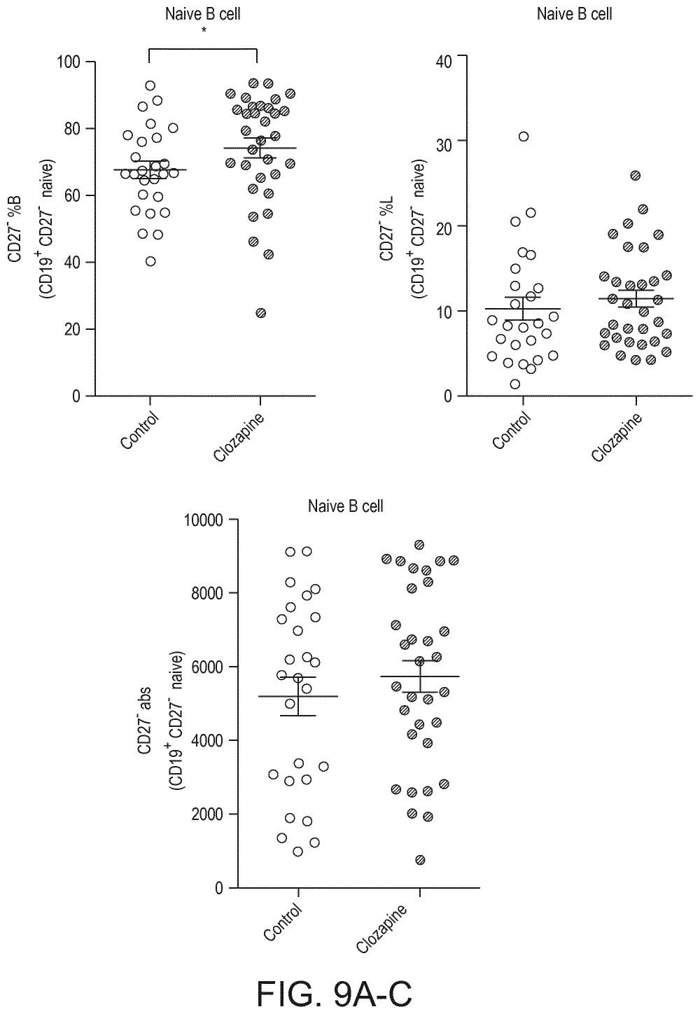

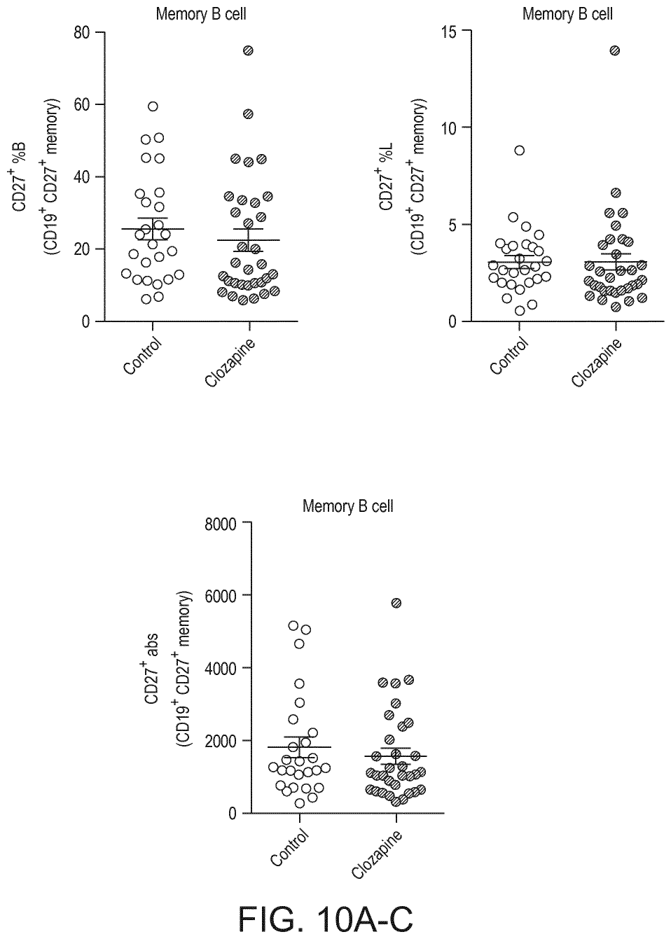

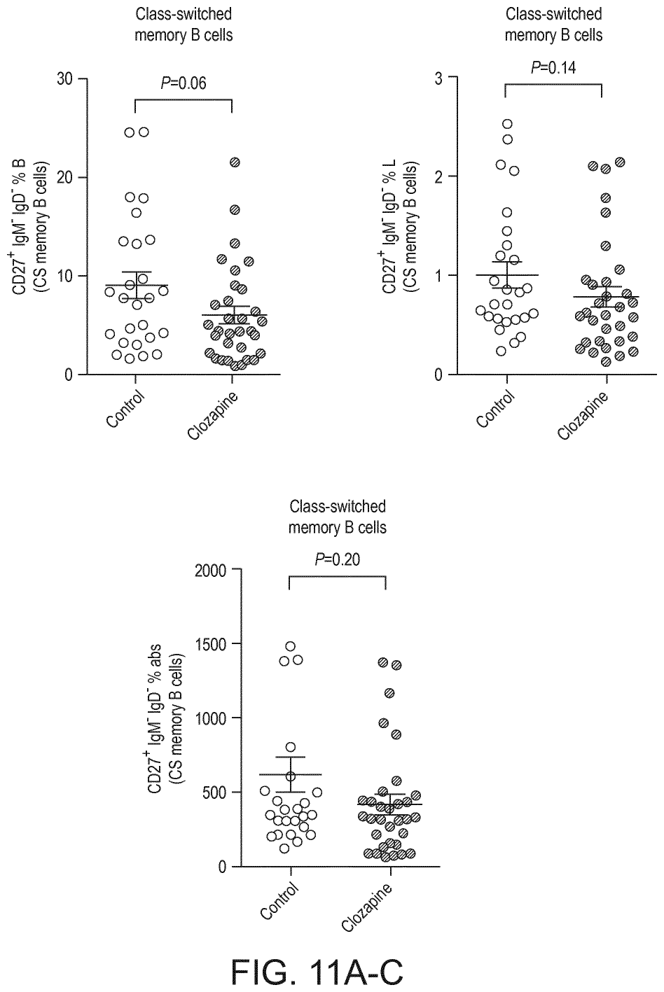

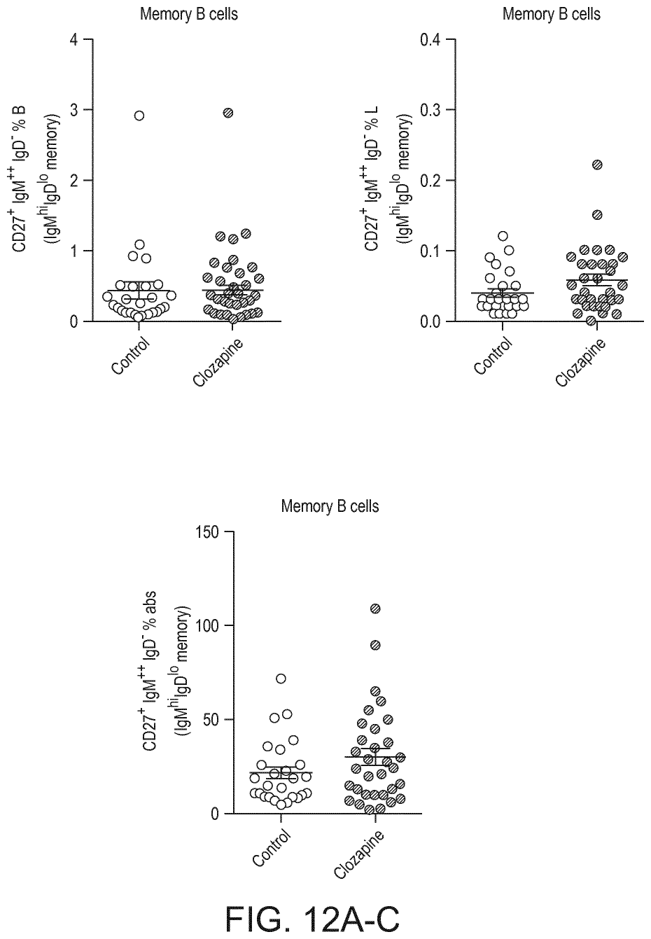

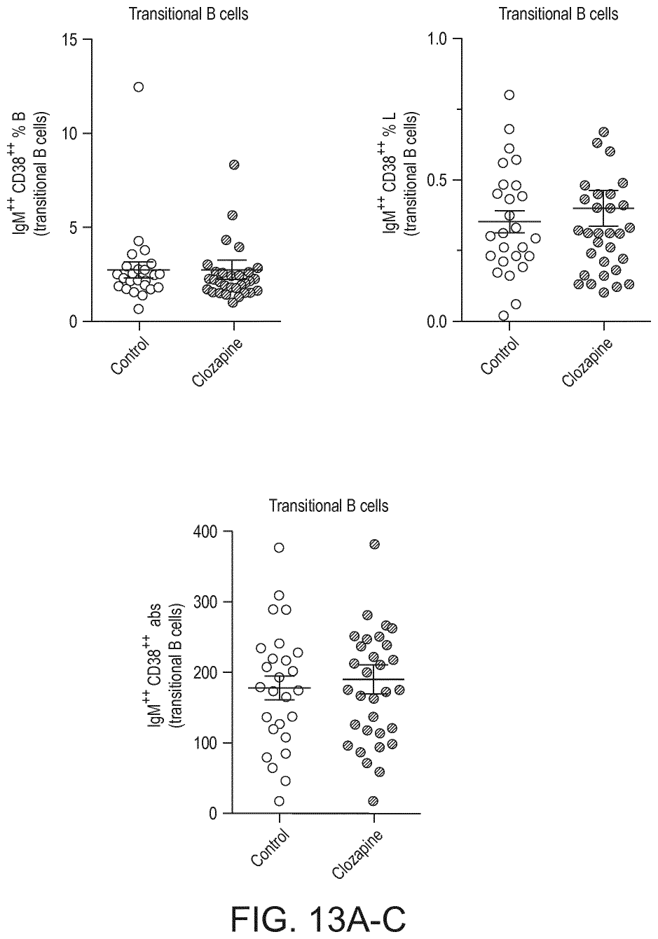

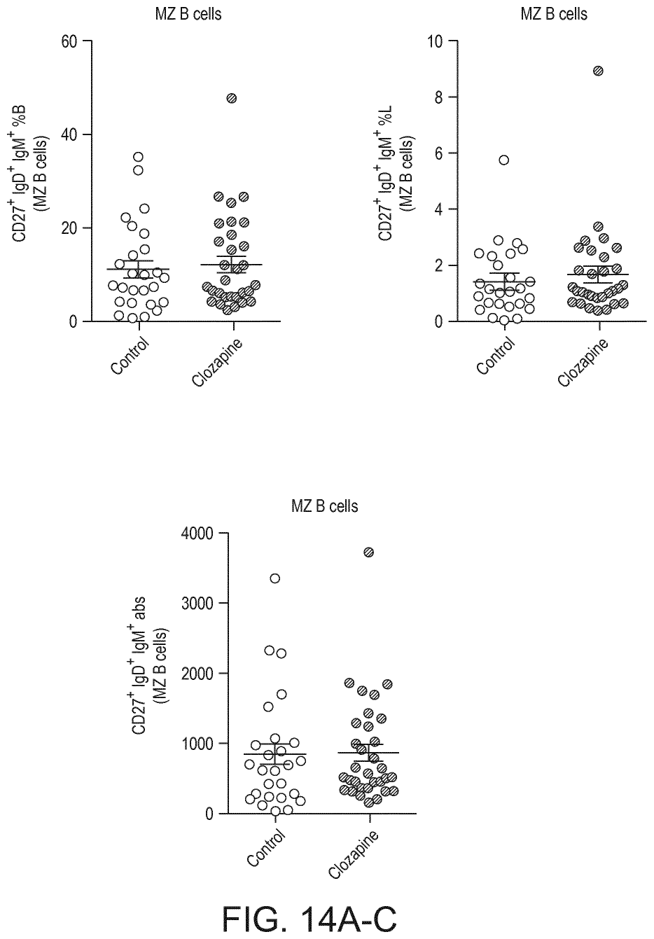

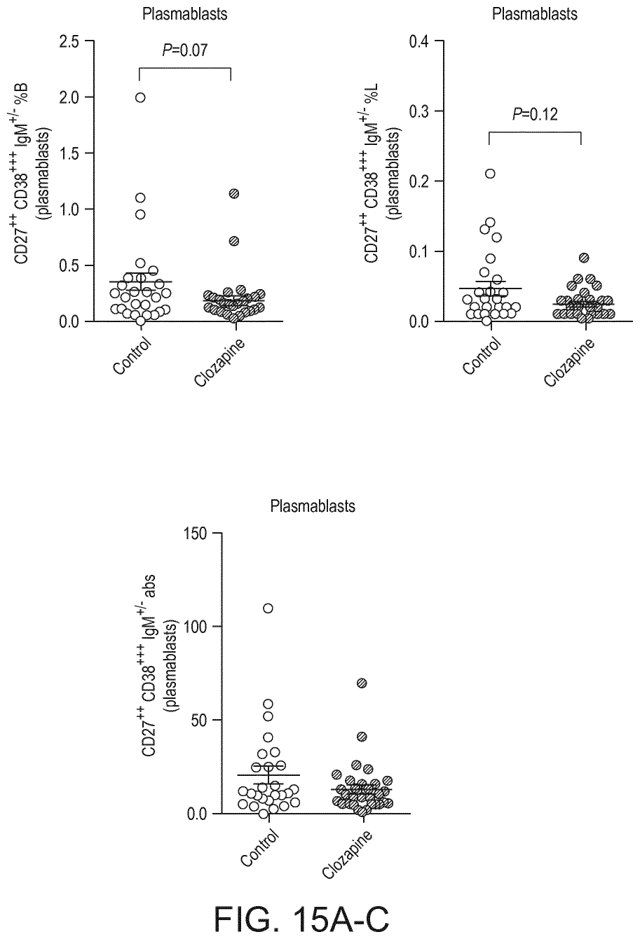

a. Significantly reduced levels of circulating total IgG, IgA and IgM in patients on clozapine versus patients who have never taken clozapine (i.e. control, clozapine naive) (see FIG. 6A-C). These reductions are relatively greater for Ig of the A and M subclass. In addition, a trend to lower IgG antibodies against pneumococcus is present in those treated with clozapine (see FIG. 7).
b. Overall CD19⁺ B cell numbers are not significantly different between groups (see FIG. 8A-B).
c. Small increase in the number of naive (CD19⁺ CD27⁻) B cells expressed as a proportion of total CD19⁺ B cells (see FIG. 9A-C).
d. Strong trends to a specific reduction in class-switched memory B cells (P=0.06 vs control, CD27⁺ IgM⁻ IgD⁻ as % B) in those treated with clozapine (see FIG. 11A-C) without perturbation of the overall memory B cell pool (see FIG. 10A-C) or IgM^{hi} IgD^{lo} memory B cell subpopulation (see FIG. 12A-C).
e. No significant difference between groups in circulating levels of transitional B cells or marginal zone B cells (See FIGS. 13A-C and 14A-C).
f. Strong trends to reduction in levels of plasmablasts in patients treated with clozapine (P=0.07 vs control clozapine naive) (see FIG. 15A-C).

### Example 3

### In Vivo Wild Type Mouse Study—Effect of Clozapine Versus Haloperidol

The impact of clozapine on B cell development, differentiation and function (inferred from circulating immunoglobulin levels) in primary (bone marrow) and secondary (spleen and also mesenteric lymph node) lymphoid tissue in wild type mice in the steady state (i.e. in the absence of specific immunological challenge) was assessed.

The specific objectives were to:

a) Determine the impact of clozapine on major B cell subsets in bone marrow and key secondary lymphoid organs (spleen and mesenteric lymph node) of healthy mice.
b) Define whether a dose-response relationship exists for clozapine on aspects of the B cell immunophenotype.
c) Assess the effect of clozapine administration on the circulating immunoglobulin profile of healthy mice.
d) Determine the specificity of clozapine's effect on the above readouts by comparison to another antipsychotic agent.

### Method

### Animals:

Young adult (age 7-8 weeks) C57BL/6 mature female mice were used for the study. Mice were housed at 22° C. in individually ventilated cages with free access to food and water and a 12-h light/dark cycle (8 a.m./8 p.m.). Mice acclimatised for 1 week on arrival prior to initiating experiments.

### Experimental Groups and Dose Selection:

Mice were allocated into one of five experimental groups as follows:

| 1. Control | saline | |
|---|---|---|
| 2. Clozapine low dose | 2.5 | mg/kg |
| 3. Clozapine intermediate dose | 5 | mg/kg |
| 4. Clozapine high dose | 10 | mg/kg |

| | |
|---|---|
| 5. Haloperidol | 1 mg/kg (intermediate dose) |

Dosing was given in staggered batches with each batch containing mice assigned to each experimental arm to reduce bias.

| | | Clozapine | Clozapine | Clozapine | Haloperidol | Mice per |
|---|---|---|---|---|---|---|
| | Control | 2.5 mg/kg | 5 mg/kg | 10 mg/kg | 1 mg/kg | batch |
| Batch 1 | 2 | 2 | 2 | 2 | 2 | 10 |
| Batch 2 | 2 | 2 | 2 | 2 | 2 | 10 |
| Batch 3 | 2 | 2 | 2 | 2 | 2 | 10 |
| Batch 4 | 2 | 2 | 2 | 2 | 2 | 10 |
| Batch 5 | 2 | 2 | 2 | 2 | 2 | 10 |
| Batch 6 | 2 | 2 | 2 | 2 | 2 | 10 |
| Mice per | 12 | 12 | 12 | 12 | 12 | 60 |
| group | | | | | | |

Dose selection was initially based on a literature review of studies administering these drugs chronically to mice (Ishisaka et al., 2015; Li et al., 2016a; Mutlu et al., 2012; Sacchi et al., 2017; Simon et al., 2000; Tanyeri et al., 2017), the great majority of which had employed the intraperitoneal (IP) route of administration: clozapine (1.5, 5, 10, 25 mg/kg/day) (Gray et al., 2009; Moreno et al., 2013); haloperidol (0.25 mg/kg, 1 mg/kg/day) (Gray et al., 2009) and taking into account the LD50 for both drugs (clozapine 200 mg/kg, haloperidol 30 mg/kg).

Subsequently, pilot studies were undertaken to assess the impact of these, particularly of the higher doses of clozapine, to refine dose selection and maximise the welfare of treated mice. Clear dose-related sedative effects were evident from dosages of clozapine starting at 5 mg/kg, with marked psychomotor suppression (with respect to depth and duration) observed at the highest doses assessed (20 mg/kg and 25 mg/kg). In addition, effects on thermoregulation were also evident, necessitating use of a warming chamber and general supportive measures to defend thermal homeostasis. These adverse effects were consistent with the known (on-target) profile of clozapine in preclinical (Joshi et al., 2017; McOmish et al., 2012; Millan et al., 1995; Williams et al., 2012) and clinical settings (Marinkovic et al., 1994), with tolerance developing after the initial few days of dosing, as has been described in humans (Marinkovic et al., 1994).

Mice (n=12/group) were treated by once daily IP injection of the respective control solution/clozapine/haloperidol for 21 consecutive days.

### Biological Samples for Immunophenotyping:

At the end of the experimental period, mice were humanely euthanised and blood samples obtained for serum separation, storage at −80° C. and subsequent measurement of immunoglobulin profiles (including the major immunoglobulin subsets IgG1, IgG2a, IgG2b, IgG3, IgA, IgM, and both light chains kappa and lambda) by ELISA.

In parallel, tissue samples were rapidly collected from bone marrow (from femur), spleen and mesenteric lymph nodes for evaluation of cellular composition across these compartments using multi-laser flow cytometric detection and analysis.

### B Cell Immunophenotyping by Flow Cytometry:

Focused B cell FACS (fluorescence-activated cell sorter) panels were prepared separately for both primary (bone marrow) and secondary (spleen/lymph node) lymphoid tissue to allow an evaluation of drug impact on the relative composition of B cell subsets spanning the spectrum of antigen-independent and -dependent phases of B cell development.

Individual antibodies employed for flow cytometry panels were pilot tested in the relevant tissues (i.e. bone marrow, spleen and mesenteric lymph node) and the optimal dilution of each antibody determined to enable clear identification of subpopulations. FACS data were extracted by BD FACSymphony and analysed by FlowJo software.

### Results

### Body Weight:

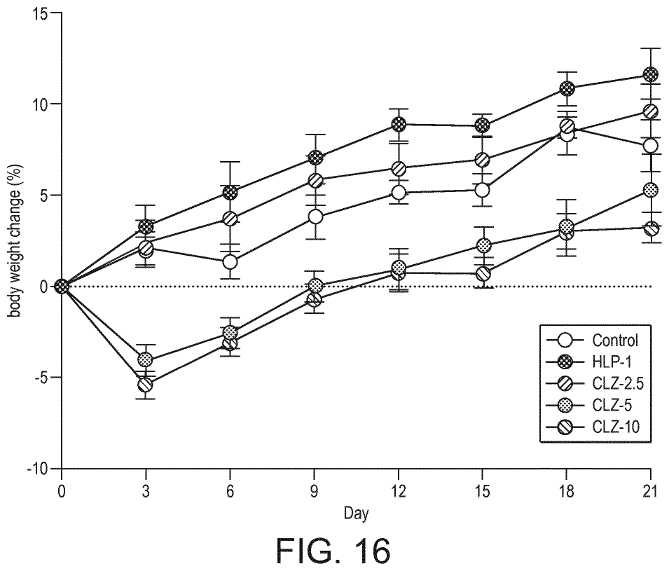

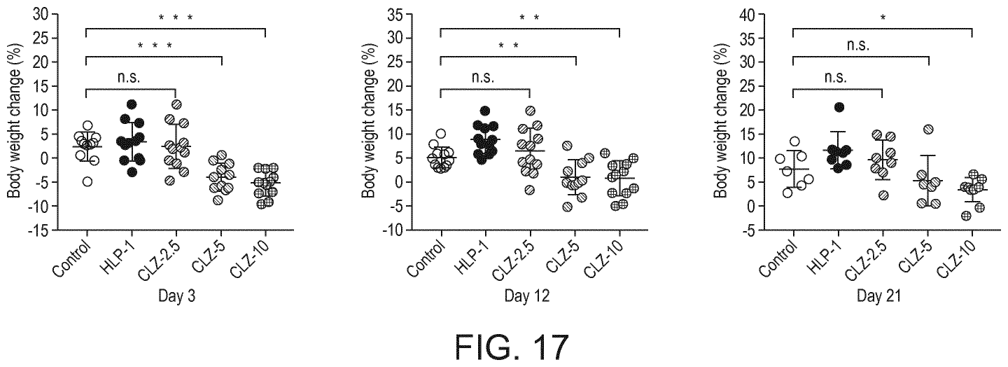

Clozapine (CLZ) induced a transient fall in body weight at both 5 mg/kg and 10 mg/kg doses, maximal by 3 days but recovering fully to baseline by day 9 with progressive weight gain beyond this (see FIGS. 16 and 17). This finding is likely to reflect the sedative effect of clozapine on fluid/food intake during the initial few days of dosing, with evidence of tolerance to this emerging over the course of the experiment.

### Early B Cell Development in Bone Marrow:

B cells originate from hematopoietic stem cells (HSCs), multipotent cells with self-renewal ability, located in the bone marrow. This early B cell development occurs from committed common lymphoid progenitor cells and progresses through a set of stages, dependent on physical and soluble chemokine/cytokine interactions with bone marrow stromal cells, defined using cell surface markers.

The earliest B cell progenitor is the pre-pro-B cell, which expresses B220 and has germline Ig genes. Next, pro-B cells rearrange their H (heavy) chain Igμ genes, and express CD19 under the control of transcription factor Pax5. At the pre-B cell stage, cells downregulate CD43, express intracellular and then rearrange the L (light) chain and upregulate CD25 in an Irf4-dependent manner. Successfully selected cells become immature (surface IgM⁺IgD⁻) B cells. Immature B cells are tested for autoreactivity through a process of central tolerance and those without strong reactivity to self-antigens exit the bone marrow via sinusoids to continue their maturation in the spleen.

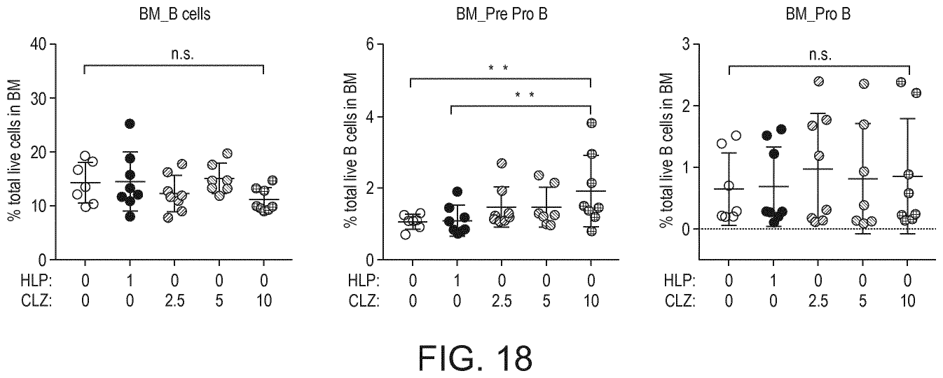

No overall reduction in B cells in the bone marrow (BM) was observed at any dose of clozapine (see FIG. 18). However, a significant increase in the proportion of very early B cell progenitors, the pre-pro B cells (i.e. B220⁺CD19⁻CD43⁺CD24^{lo}BP-1⁻IgM⁻IgD⁻) was observed with 10 mg/kg clozapine, without any change evident in the subsequent pro-B cell fraction (see FIG. 18). In contrast, no significant effect of haloperidol was evident on any of these early developing B cell subsets.

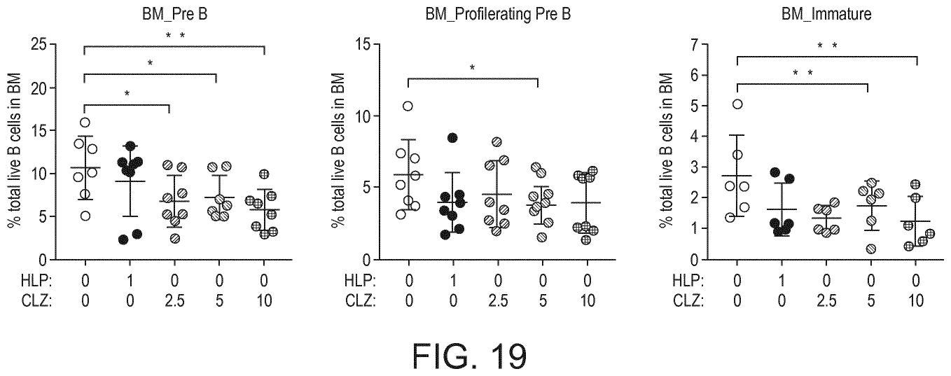

Examination of subsequent stages of B cell development in bone marrow revealed a reduction in pre-B cells (i.e. B220⁺CD19⁺CD43⁻CD24⁺BP-1⁻IgM⁻IgD⁻) in mice treated with clozapine (see FIG. 19). Notably this effect exhibited dose-dependency, with a significant difference observed verses control mice with even the lowest dose of clozapine employed (2.5 mg/kg). Furthermore, the percentage of pre-B cells that were proliferating (i.e. B220⁺CD19⁺CD43⁻CD24^{hi}BP-1⁺I⁺IgM⁻IgD⁻) was diminished with clozapine, reaching significance for the 5 mg/kg dose (see FIG. 19). Correspondingly, a reduction in the percentage of immature B cells in bone marrow was identified (i.e. B220⁺CD19⁺CD43⁻CD24⁺IgM⁺IgD⁻) (see FIG. 19).

Together, these findings suggest a specific impact of clozapine on early B cell development, with a modest arrest between the pre-pro-B cell and pre-B cell stages in the absence of specific immunological challenge.

### Peripheral B Cell Development—Total Splenic B Cells:

After emigrating from the bone marrow, functionally immature B cells undergo further development in secondary lymphoid organs, enabling further exposure to (peripheral) self-antigen and peripheral tolerance (resulting in cell deletion through apoptosis, anergy or survival). The majority of immature B cells exiting bone marrow do not survive to become fully mature B cells, a process regulated by maturation and survival signals received in lymphoid follicles, including BAFF (B cell activating factor) secreted by follicular dendritic cells.

Mice treated with clozapine at 5 mg/kg and 10 mg/kg were seen to have a significantly lower percentage of splenic B cells (i.e. B220⁺TCR-β⁻) expressed as a proportion of total live splenocytes (see FIG. 21). No effect was identified on other cell populations (i.e. B220⁻TCR-β⁻, which may include γδ T cells (which do not express the αβ T cell receptor, TCR), natural killer (NK) cells, or other rare lymphoid cell populations (see FIG. 21). This was accompanied by a reciprocal increase in the percentage of splenic T cells (i.e. B220-TCR-β+) (see FIG. 21). In contrast, activated T cells (i.e. B220⁺TCR-β⁺), reflecting a small proportion of total live splenocytes were reduced in dose-dependent fashion by clozapine compared to control, an effect also modestly apparent for haloperidol (see FIG. 21).

These findings suggest that clozapine, but not haloperidol, is able to affect peripheral (splenic) B cells in addition to the observed changes in bone marrow B cell precursors.

### Splenic B Cell Subpopulations:

Immature B cells exiting the bone marrow and entering the circulation are known as transitional B cells. These immature cells enter the spleen and competitively access splenic follicles to differentiate via transitional stages to immunocompetent naive mature B cells. This occurs sequentially in the follicle from transitional type 1 (T1) cells, similar to immature B cells in bone marrow, to type 2 (T2) precursors. The latter are thought to be the immediate precursor of mature naive B cells. T2 B cells have been demonstrated to show greater potency in response to B cell receptor stimulation than T1 B cells, suggesting that the T2 subset may preferentially undergo positive selection and progression into the long-lived mature B cell pool (Petro et al., 2002).

Transitional cells can differentiate into follicular B cells, representing the majority of peripheral B cells residing in secondary lymphoid organs, or a less numerous population, marginal zone (MZ) B cells residing at the white/red pulp interface which are able to respond rapidly to blood-borne antigens/pathogens.

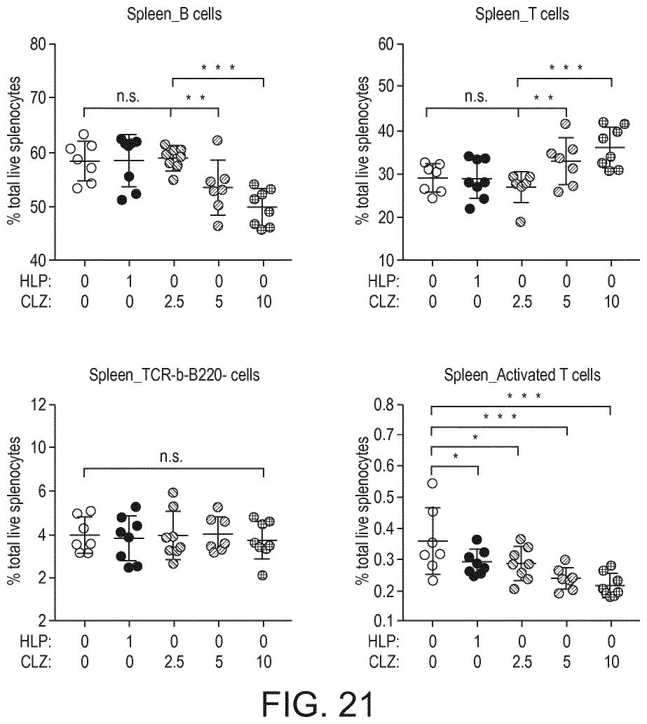

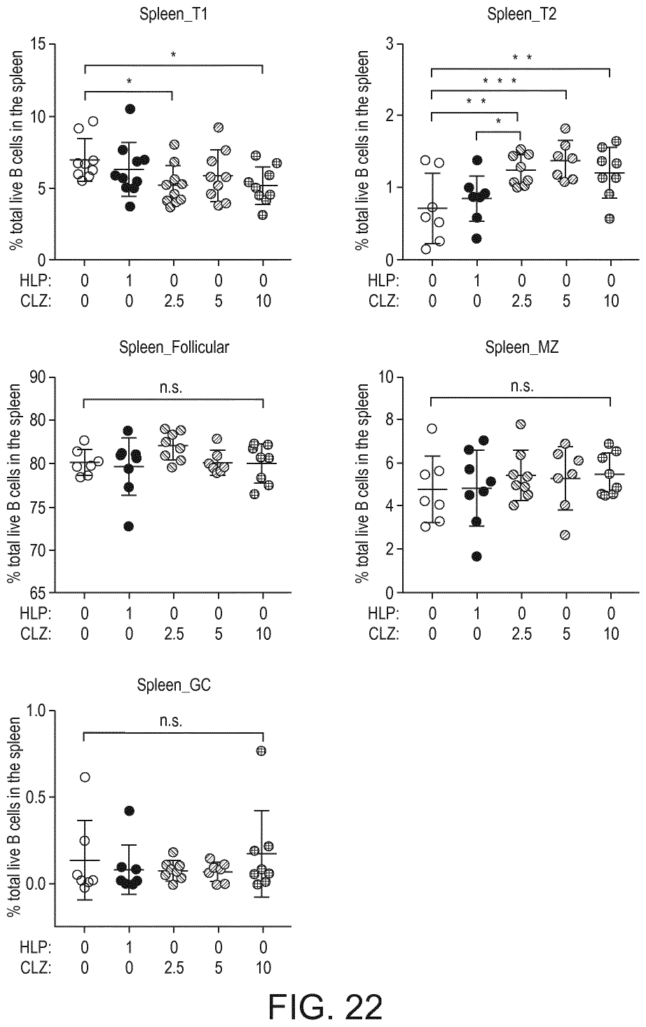

Mice treated with clozapine were found to have a mildly reduced proportion of newly emigrated transitional stage 1 (T1) B cells in the spleen, including at the 2.5 mg/kg dose, which may in part reflect the reduction in percentage of bone marrow immature B cells (see FIG. 22). In contrast, a small increase in the proportion of T2 B cells was identified across all doses of clozapine (see FIG. 22), consistent with enhanced positive selection of T1 B cell subsets for potential progression into the long-lived mature B cell pool.

While clozapine administration reduced the splenic B cell contribution to live splenocytes (see FIG. 21), no specific reductions were identified in either splenic follicular (i.e. B220⁺CD19⁺CD21^{mid}CD23⁺) or marginal zone (i.e. B220⁺CD19⁺CD21⁺CD23^{Lo/-}) B cell subsets (see FIG. 22), suggesting that in the immunologically unchallenged state, clozapine administration in mice results in a global reduction in splenic B cell populations.

Germinal centres (GCs) are micro-anatomical structures which form over several days in B cell follicles of secondary lymphoid tissues in response to T cell-dependent antigenic (e.g. due to infection or immunisation) challenge (Meyer-Hermann et al., 2012). Within GCs, B cells undergo somatic hypermutation of their antibody variable regions, with subsequent testing of the mutated B cell receptors against antigens displayed by GC resident follicular dendritic cells. Through a process of antibody affinity maturation, mutated B cells which higher affinity to antigen are identified and expanded. In addition, class switch recombination of the immunoglobulin heavy chain locus of mature naive (IgM⁺IgD⁺) B cells occurs before and during GC reactions, modifying antibody effector function but not its specificity or affinity for antigen. This results in isotype switching from IgM to other immunoglobulin classes (IgG, IgA or IgE) in response to antigen stimulation.

GCs are therefore sites of intense B cell proliferation and cell death, with outcomes including apoptosis, positive selection for a further round of somatic hypermutation (i.e. cyclic re-entry), or B cell differentiation into antibody secreting plasma cells and memory B cells (Suan et al., 2017). In the steady state, GC cells (i.e. B220+CD19+IgD−CD95+GL−7+) formed a very small proportion of total live B cells in the spleen, with no differences observed versus control or haloperidol in response to clozapine administration (see FIG. 22).

### Bone Marrow Antibody Secreting Cell Populations:

Antibody secreting cells represent the end-stage differentiation of the B cell lineage and are widely distributed in health across primary and secondary lymphoid organs, the gastrointestinal tract and mucosa (Tellier and Nutt, 2018). These cells all derive from activated B cells (follicular, MZ or B1). Plasmablasts, representing short-lived cycling cells, can be derived from extra-follicular differentiation pathway in a primary response (producing relatively lower affinity antibody), as well as from memory B cells that have undergone affinity maturation in the GC (Tellier and Nutt, 2018).

Plasmablasts developing in GCs can leave the secondary lymphoid organ and home to the bone marrow. Here, only a small proportion are thought to be retained and establish themselves in dedicated micro-environmental survival niches to mature into long-lived plasma cells (Chu and Berek, 2013), a process thought to be regulated by docking onto mesenchymal reticular stromal cells (Zehentmeier et al., 2014) and requiring haematopoietic cells (e.g. eosinophils) (Chu et al., 2011a), the presence of B cell survival factors (e.g. APRIL and IL-6) (Belnoue et al., 2008) and hypoxic conditions (Nguyen et al., 2018).

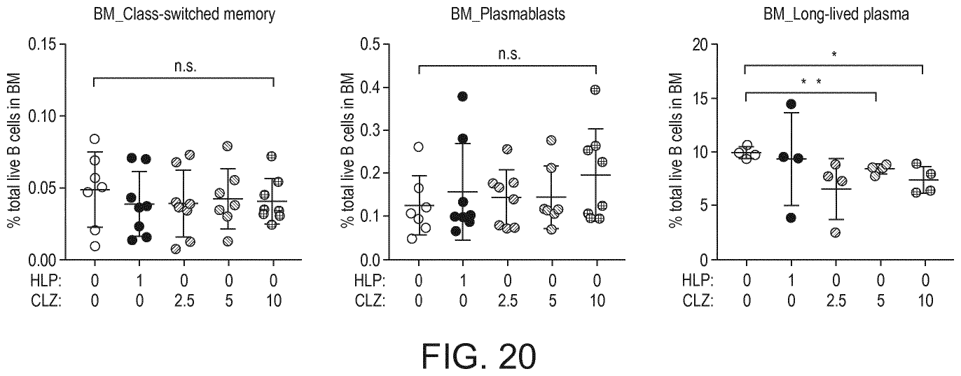

In the healthy state, the bone marrow houses the majority of long-lived plasma cells. Clozapine at 5 and 10 mg/kg induced a significant reduction in the percentage of long-lived plasma cells in the bone marrow (i.e. B220^{lo}CD19⁻IgD⁻IgM⁻CD20⁻CD38⁺⁺CD138⁺) by ^{˜}30% compared to control (see FIG. 20). In contrast, no effect of haloperidol was seen on this specific B cell population (see FIG. 20). No significant changes were detected in either class-switched memory B cells (i.e. B220⁺CD19⁺CD27⁺IgD⁻IgM⁻CD20⁺CD38^{+/-}) or plasmablasts (i.e. B220^{lo}CD19⁺CD27⁺IgD⁻IgM⁻CD20⁻CD38⁺⁺) in the bone marrow with any treatment, however both these represent a very small proportion of total B cells in the bone marrow in the immunologically unchallenged steady state (see FIG. 20).

These findings indicate that clozapine can exert a specific effect to reduce the proportion of long-lived plasma cells in the bone marrow, a population thought to be the major source of stable antigen-specific antibody titres in plasma involved in humoral immune protection and, in pathogenic states, stable autoantibody production.

### Circulating Immunoglobulin Levels:

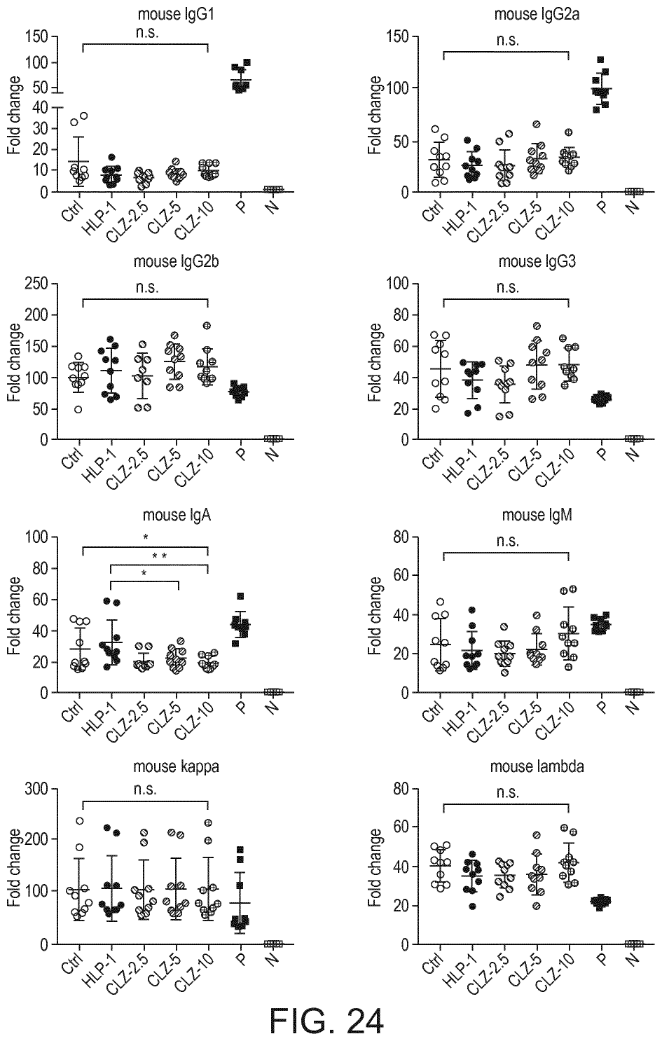

Clozapine administration at both 5 and 10 mg/kg resulted in a reduction in circulating IgA levels compared to control, an effect not observed with haloperidol (see FIG. 24; P, positive control; N, negative control). No other isotype classes were affected under the experimental conditions used (see FIG. 24).

### Mesenteric Lymph Nodes:

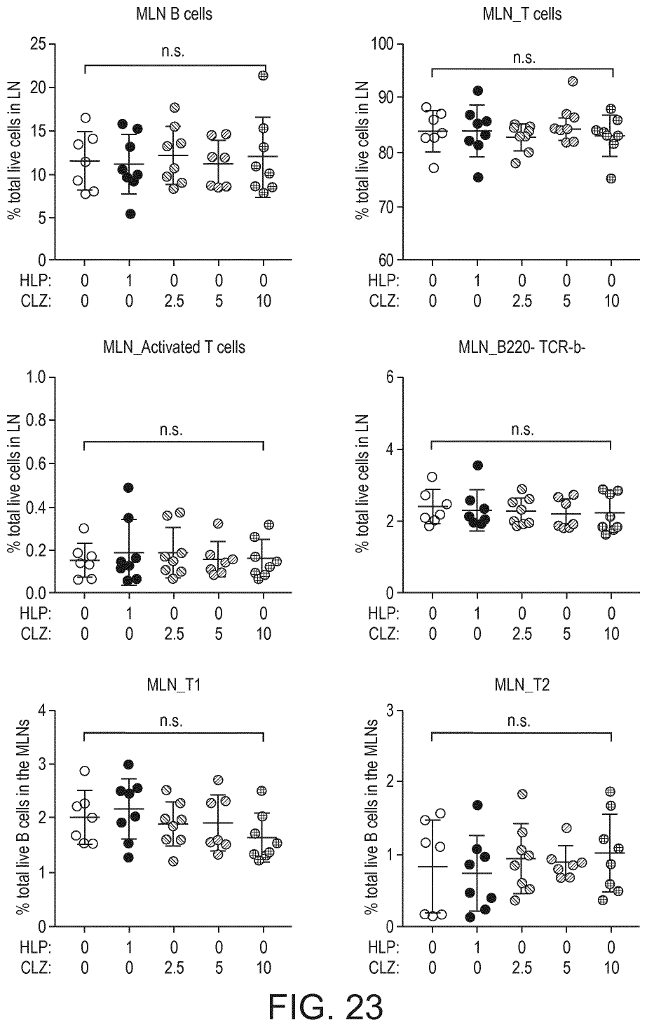

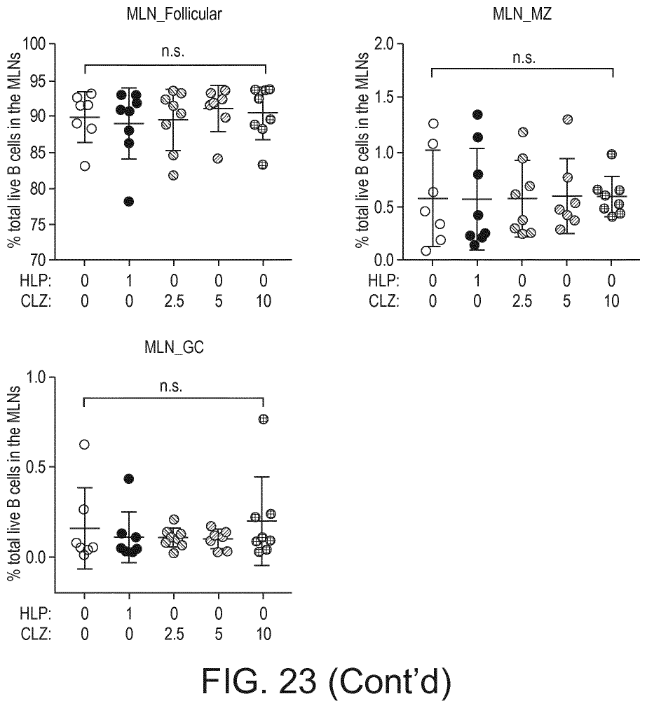

Under the current experimental conditions, no significant differences were identified between any of the groups in lymphocyte subpopulations assessed in mesenteric lymph nodes (MLN) (see FIG. 23).

### Conclusion

This study investigated the potential for clozapine to influence the immunophenotype of wild type mice in the steady state, specifically B cell subpopulations, with functional impact inferred through circulating levels of immunoglobulins. The major findings of this study are that 3 weeks parenteral (I.P.) administration of clozapine:

a) Increases the proportion of pre-pro-B cells while reducing the proportion of later-stage pre-B cells and immature B cells in the bone marrow.
b) Reduces the proportion of live splenocytes that are B cells.
c) Exerts subtle effects on developing B cells in the spleen, specifically transitional B cell populations in favouring a greater proportion of T2 type cells.
d) Significantly reduces the proportion of long-lived plasma cells in the bone marrow.
e) Impacts on circulating immunoglobulin levels, specifically lowering IgA.
f) Results in a dose-dependent decrease in the proportion of activated T cells in spleen which, in contrast to all the above findings, was also observed with the dose of haloperidol used.

Taken together, these observations indicate that clozapine exerts complex effects on B cell maturation in vivo, not limited to the late stages of B cell differentiation or activation. Specifically, the findings suggest that clozapine can influence the maturation of early B cell precursors, with a partial arrest of antigen-independent B cell development in the bone marrow.

In parallel, clear effects of clozapine are identified on peripheral B cell subpopulations, with a notable impact on reducing the overall B cell proportion of live splenocytes, and on long-lived antibody secreting plasma cells in the bone marrow. An impact on antibody secreting cells is likely to underlie the observed significant reduction in circulating IgA, particularly striking given the otherwise immunologically unchallenged state of the mice.

Notably, the impact on B cell subpopulations was not observed with a comparator antipsychotic agent, haloperidol, consistent with specificity of action of clozapine on B cell maturation. While the current experiments do not enable a distinction between a direct or indirect effect of clozapine on bone marrow, peripheral and late B cell populations, taken together with findings from separate in vitro B cell proliferation assays, an indirect effect is deemed more likely. This may involve a variety of other myeloid, lymphoid (e.g. T follicular helper cells) and/or (mesenchymal) stromal supportive cells.

### Example 4

### Mouse Collagen-Induced Arthritis (CIA) Model Study—Effect of Clozapine

The CIA model is a well-established experimental model of autoimmune disease. The inventors have employed the CIA model as a highly clinically relevant experimental system in which B cell-derived pathogenic immunoglobulin made in response to a sample antigen drives autoimmune pathology to explore the potential efficacy of clozapine and its associated cellular mechanisms.

### Method

### Animals:

Adult (age 13-15 weeks) DBA/1 male mice were purchased from Envigo (Horst, Netherlands). Mice were housed at a 21° C.±2° C. in individually ventilated cages with free access to food and water and a 12-h light/dark cycle (7 am/7 pm). Mice were acclimatised for 1 week on arrival prior to initiating experiments.

### Experimental Groups and Dose Selection:

Mice were allocated into one of five experimental groups as follows:

1. Control saline
2. Clozapine 5 mg/kg treatment from day 15 after immunization
3. Clozapine 10 mg/kg treatment from day 15 after immunization
4. Clozapine 5 mg/kg treatment from day 1 after immunization
5. Clozapine 10 mg/kg treatment from day 1 after immunization

Mice (n=10/group) were treated by once daily IP injection of the respective control solution/clozapine until day 10 after onset of clinical features of arthritis. All experiments were approved by the Clinical Medicine Animal Welfare and Ethical Review Body (AWERB) and by the UK Home Office.

### Anti-Arthritic Effect of Clozapine In Vivo:

DBA/1 mice were immunised with bovine type II collagen in CFA and monitored daily for onset of arthritis. Clozapine was administered daily by intraperitoneal injection at doses of 5 mg/kg or 10 mg/kg. Controls received vehicle (saline) alone. Treatment of mice commenced in one experiment on day 1 after immunisation and in a second experiment on day 15 after immunisation. Clinical scores and paw-swelling were monitored for 10 days following onset of arthritis. A clinical scoring system was used as follows. Arthritis severity was scored by an experienced, non-blinded investigator as follows: 0=normal, 1=slight swelling and/or erythema, 2=pronounced swelling, 3=ankylosis. All four limbs were scored, giving a maximum possible score of 12 per animal.

At the end of the experimental period, mice were humanely euthanised and bled by cardiac puncture to obtain blood samples for serum separation, storage at −80° C. and subsequent measurement of specific anti-collagen immunoglobulin (IgG1 and IgG2a isotypes) by ELISA. In parallel, spleen and inguinal lymph nodes were harvested for evaluation of cellular composition across these compartments using multi-laser flow cytometric detection and analysis. Numbers of B cell subsets in spleen and lymph nodes were determined by FACS.

### Statistical Analysis:

Data were analyzed by one-way ANOVA with Tukey's or Dunnett's multiple comparison test or two-way ANOVA with Tukey's multiple comparison test as appropriate. All calculations were made using GraphPad Prism software. A P value less than 0.05 was considered significant.

### Results

### Effect of Clozapine on Onset, Clinical Score and Paw-Swelling:

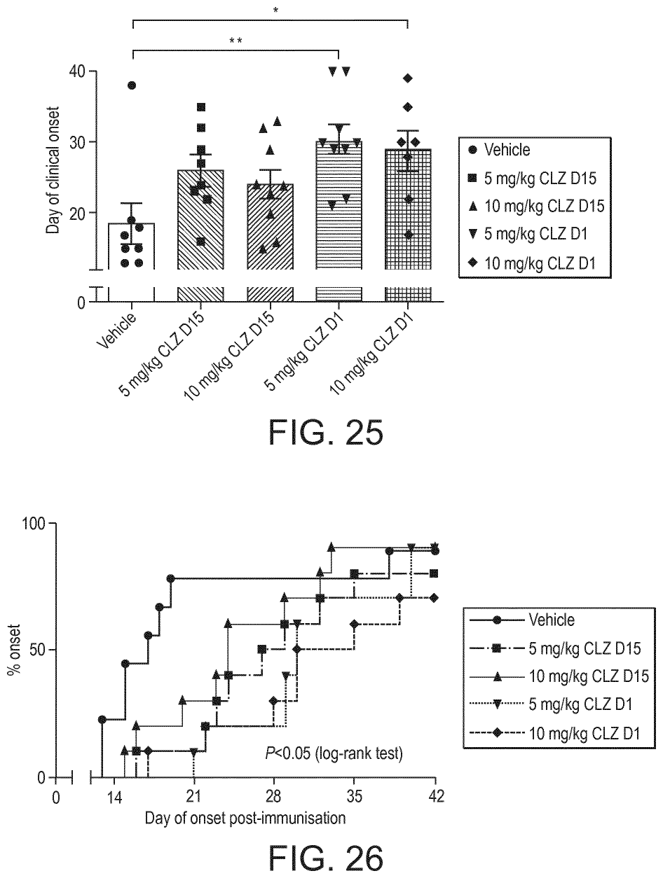

Treatment of mice with clozapine was significantly effective in delaying the onset of arthritis post-immunisation (see FIGS. 25 and 26). In particular, treatment with both doses of clozapine from day 1 was extremely effective in delaying arthritis onset (see FIGS. 25 and 26).

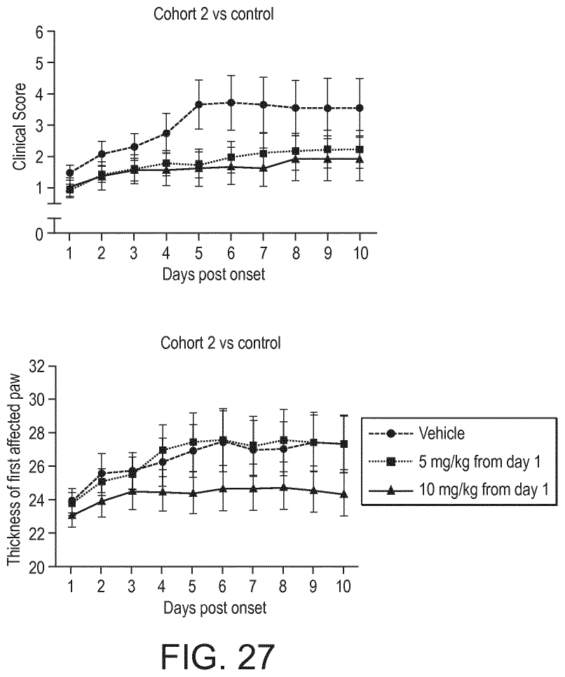

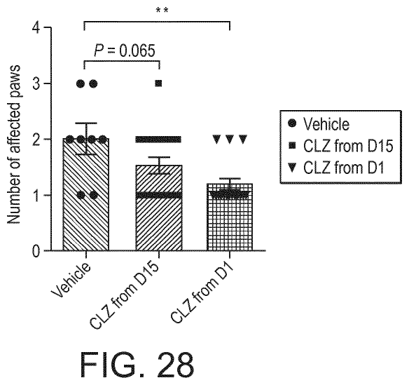

Furthermore, treatment with both doses of clozapine reduced overall clinical score when administered on day 1 and, in the case of 10 mg/kg clozapine, also reduced swelling of the first affected paw (see FIG. 27). Clozapine administration also reduced the total number of affected paws compared to vehicle control, an effect significant with dosing at D1 (see FIG. 28).

### Effect of Clozapine on Peripheral B Cell Subsets:

Mice treated with clozapine at all doses and time points (i.e. 5 mg/kg or 10 mg/kg from day 1 or day 15) were seen to have a significantly lower percentage of B220⁺ B cells in lymph nodes (see FIG. 29). In addition, clozapine administered at 10 mg/kg from day 1 also significantly reduced the proportion of B220⁺ B cells in spleen.

Under the experimental conditions employed, no significant effect of clozapine was observed on plasma cell numbers in lymph node, however a significant reduction in the proportion of plasma cells was identified in spleen at a dose of 10 mg/kg clozapine given on day 1, with nominally lower values for plasma cells as a proportion of live cells at every other dose/time evaluated compared to control (see FIG. 30).

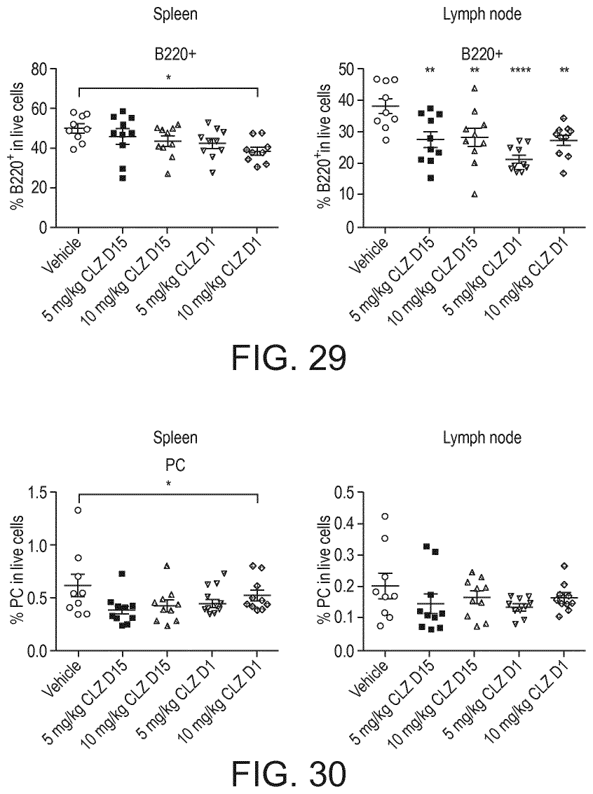

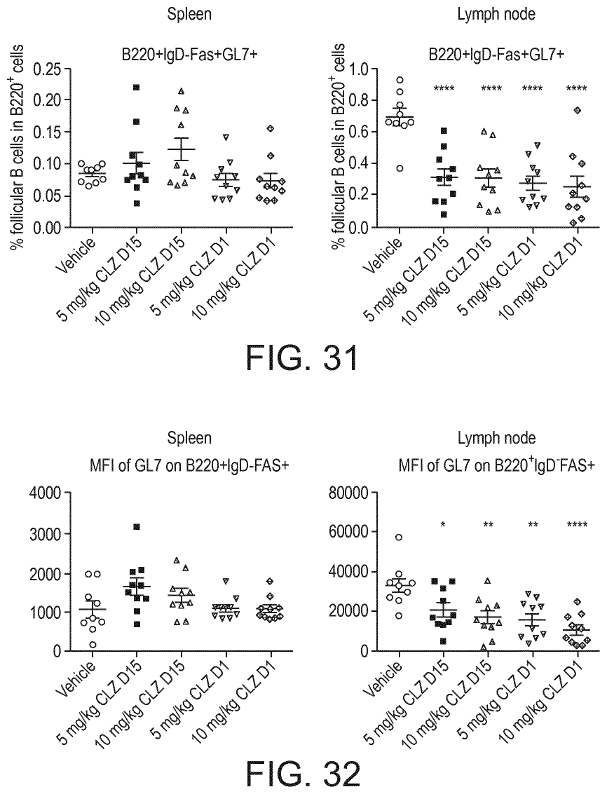

Strikingly significant reductions in lymph node follicular B cells (B220⁺IgD⁻Fas⁺GL7^{hi}) were observed in mice treated with clozapine across all doses/both time points (see FIG. 31). In addition, the level of GL7 expression on follicular B cells in lymph node were significantly decreased across all clozapine treatment groups compared to vehicle treated controls (see FIG. 32). There was evidence of dose- and time-dependency of effect with particularly profound reductions in GL7 epitope expression in mice treated with clozapine from day 1 (see FIG. 32).

### Effect of Clozapine on Anti-Type II Collagen IgG Isotypes:

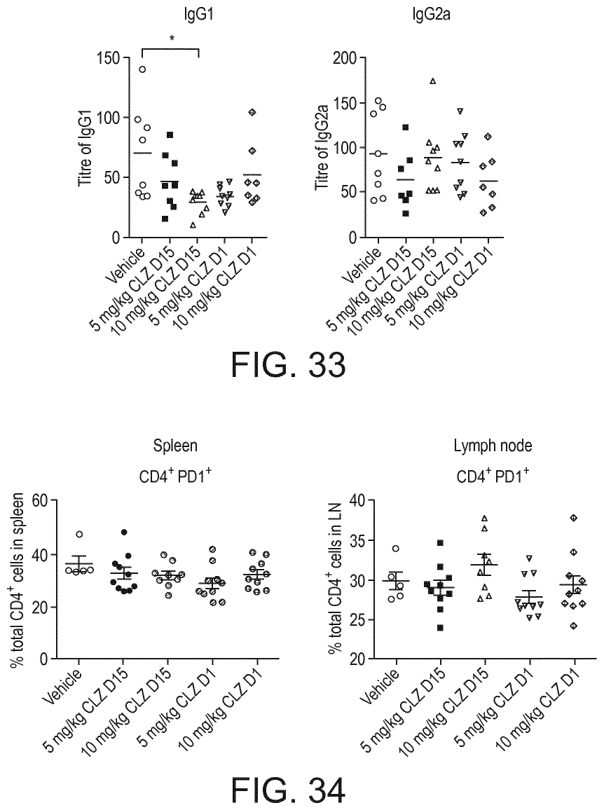

Clozapine administration at 5 or 10 mg/kg from day 1 or day 15 had no significant impact on serum IgG2a measured at a single time point. However, clozapine administration led to nominal reductions in levels of IgG1 across all doses tested, reaching statistical significance for the group treated with 10 mg/kg from day 15 (see FIG. 33).

### Effect of Clozapine on T Follicular Helper Cells:

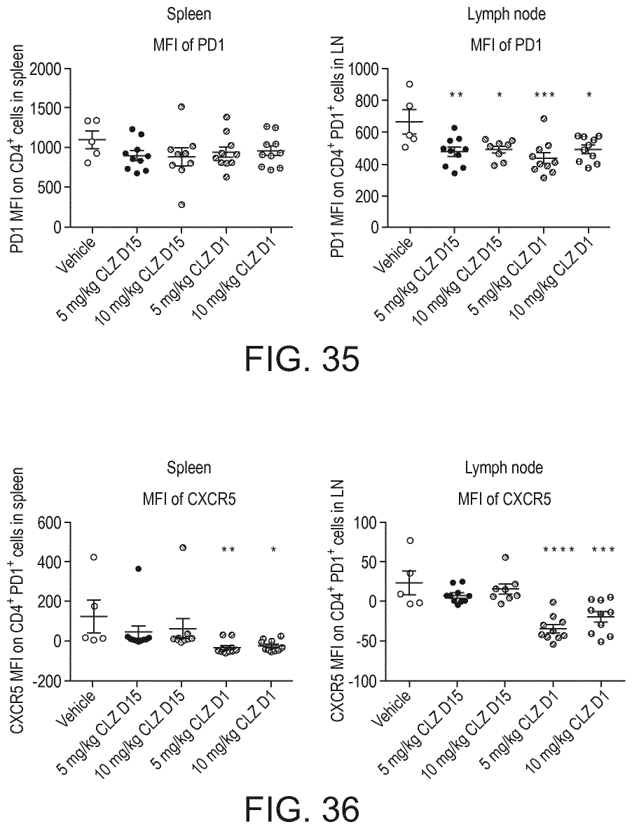

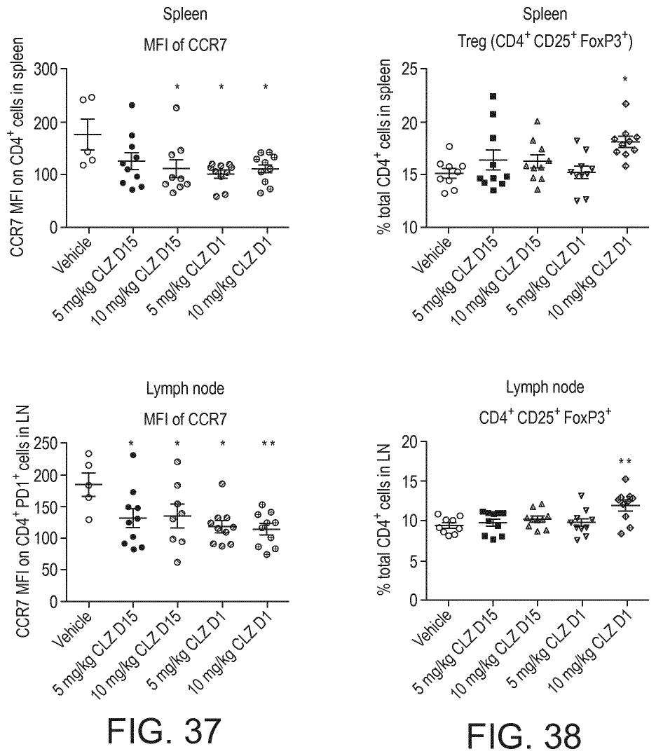

Treatment of mice with 5 mg/kg or 10 mg/kg of clozapine from day 1 or day 15 did not significantly affect proportions of CD4⁺PD1⁺CXCR5⁺ T follicular helper cells in lymph node or spleen (see FIG. 34). However, analysis of mean fluorescence intensity (MFI) revealed robust reductions in expression of PD-1 and CXCR5 on T follicular helper cells in mice-treated with clozapine (see FIGS. 35 and 36). Reduced expression of PD-1 in lymph node T follicular helper cells was evident for clozapine at all doses and time points evaluated (see FIG. 35). In the case of CXCR5 expression, significant reductions were observed in mice dosed with clozapine from day 1 and evident in both lymph node (strongest signal for reduction) and spleen (see FIG. 36). In addition, reduced expression of CCR7 on T follicular helper cells was observed in mice treated with clozapine both in lymph node and in spleen (see FIG. 37).

### Effect of Clozapine on T Regulatory Cells:

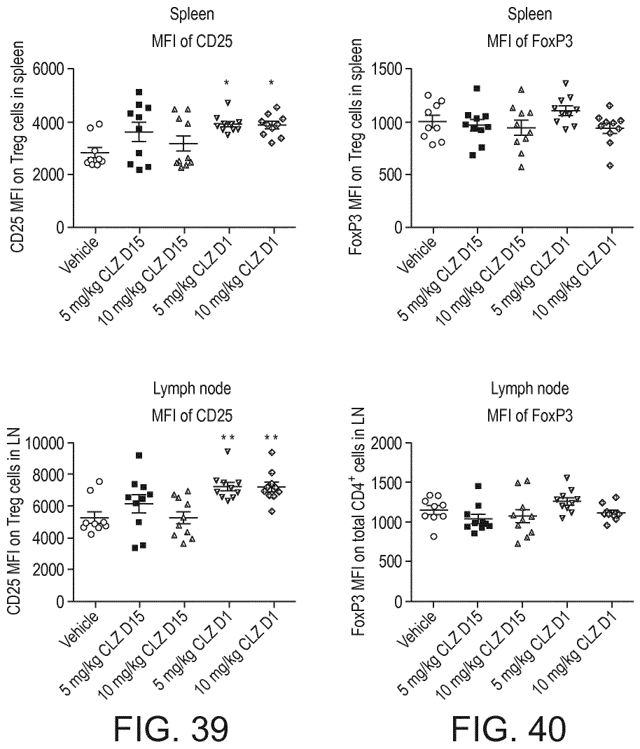

When used at the higher dose tested and from day 1 after immunisation, clozapine was seen to increase the proportion of CD4⁺CD25⁺Foxp3⁺ T regulatory cells (Tregs) in both lymph node and spleen (See FIG. 38). In addition, clozapine when dosed from day 1 was seen to significantly upregulate the expression of CD25 on these cells (see FIG. 39), but not alter Foxp3 expression itself (see FIG. 40).

### Conclusion

This study investigated the potential for clozapine to ameliorate CIA and its impact on major B cell subsets. The major findings of this study are as follows.

a) Clozapine is extremely effective at delaying disease onset in the CIA model.
b) Clozapine ameliorates the severity in CIA.
c) Clozapine reduces the proportion of B220⁺ B cells in both spleen and lymph node.
d) Clozapine reduces the proportion of splenic plasma cells.
e) Clozapine results in substantial reduction in the proportion of lymph node follicular B cells (IgD⁻Fas⁺GL7^{hi}) in B220⁺ B cells and lowers their expression of GL-7.
f) Clozapine demonstrated some ability to reduce pathogenic immunoglobulin, specifically anti-collagen IgG1 (at a dose of 10 mg/kg dosed from D15 after immunisation) in the context of the experimental conditions assessed (single time point immunoglobulin measurement).
g) Clozapine markedly reduces the expression of PD1 and CXCR5, in addition to CCR7, on lymph node T follicular helper cells (PD1⁺CXCR5⁺) without impacting upon the proportion of cells.

Taken together, these observations indicate that clozapine delayed disease onset, probably through multiple mechanisms likely to involve its impact on (secondary) lymphoid tissue and its ability to form functional germinal centres with subsequent impact on antibody producing B cells.

Specifically, clozapine is seen to reduce germinal centre B cells in local lymph node [marked by expression of GL7 in immunised spleen/lymph node (Naito et al., 2007)] following immunisation. GL7^{hi} B cells exhibit higher specific and total immunoglobulin production in addition to higher antigen-presenting capacity (Cervenak et al., 2001). Thus the observation of a reduction in surface expression of the GL7 epitope with clozapine suggests an impact to lower functional activity of these B cells for producing antibody and presenting antigen.

In parallel, clozapine is seen to affect T follicular helper cells, a critical T cell subset which controls the formation of and coordinates the cellular reactions occurring within germinal centres that is essential for somatic hypermutation, isotype class switching and antibody affinity maturation, differentiating B cells into memory B cells or plasma cells. T follicular helper cells therefore specialise in promoting the T cell-dependent B cell response (Shi et al., 2018). In particular, while not affecting the overall proportion of T follicular helper cells, clozapine is seen to reduce PD1 (programmed cell death-1) expression which is essential for proper positioning of T follicular helper cells through promoting their concentration into the germinal centre from the follicle (Shi et al., 2018). PD1 is also required for optimal production of IL-21 by T follicular helper cells, with PD1-PD-L1 interactions (i.e. the cognate ligand of PD1) between T follicular helper cells and germinal centre B cells aiding the stringency of affinity-based selection.

Furthermore, clozapine was seen to reduce the expression of CXCR5 on T follicular helper cells. CXCR5 (CXC chemokine receptor 5) is regarded as the defining marker for these cells; upregulation of CXCR5 enables relocation to the T/B border and, through attraction to CXCL-13, the B cell zone of lymphoid tissue to allow T follicular helper cells to enter the B cell follicle (Chen et al., 2015). Accordingly, reduced expression of CXCR5 on T follicular helper cells would impede their migration into B cell follicles and thereby reduce their ability to localise and interact with germinal centre B cells. Consistent with this, mice deficient in CXCR5 or selectively lacking CXCR5 on T cells display complete resistance to induction in CIA, in concert with reduced secondary lymphoid germinal centre formation and lower anti-collagen antibody production (Moschovakis et al., 2017).

Clozapine was also found to reduce expression of CCR7 on T follicular helper cells. CCR7 downregulation is regarded as an important mechanism through which activated CD4⁺ T cells overcome T zone chemokines which promote retention in the T zone (Haynes et al., 2007). Importantly, promotion of normal germinal centre responses by T follicular helper cells requires a coordinate upregulation of CXCR5 and downregulation of CCR7 (Haynes et al., 2007). Thus, the balanced expression of CXCR5 and CCR7 is critical to fine tuning of T follicular helper cell positioning and efficient provision of B cell help (Hardtke et al., 2005). The observation that clozapine can influence both CXCR5 and CCR7 expression on T follicular helper cells is therefore consistent with an ability of clozapine to perturb positioning and proper function of these cells, vital for T cell support of production of high affinity antibodies in response to T dependent antigens.

Further highlighting the importance of germinal centre formation to the pathogenesis of CIA is the finding that syndecan-4 null mice, which exhibit lower numbers of B cells and deficient germinal centre formation in draining lymph nodes, are resistant to CIA (Endo et al., 2015).Given the critical importance of tight regulation of germinal centres to the maintenance of self-tolerance and prevention of pathogenic autoantibody production in autoimmunity, the impact of clozapine as demonstrated in the CIA model strongly supports its potential to mitigate pathogenic autoantibody production.

### Example 5

### Study of Effect of Clozapine and Norclozapine on Human Plasma Cell Generation Using an In Vitro B Cell Differentiation System

An established in vitro platform (Cocco et al., 2012) was used to evaluate the impact of clozapine, its major metabolite norclozapine and a comparator antipsychotic drug, haloperidol, on the generation and differentiation and viability of human plasma cells.

### Method

### General:

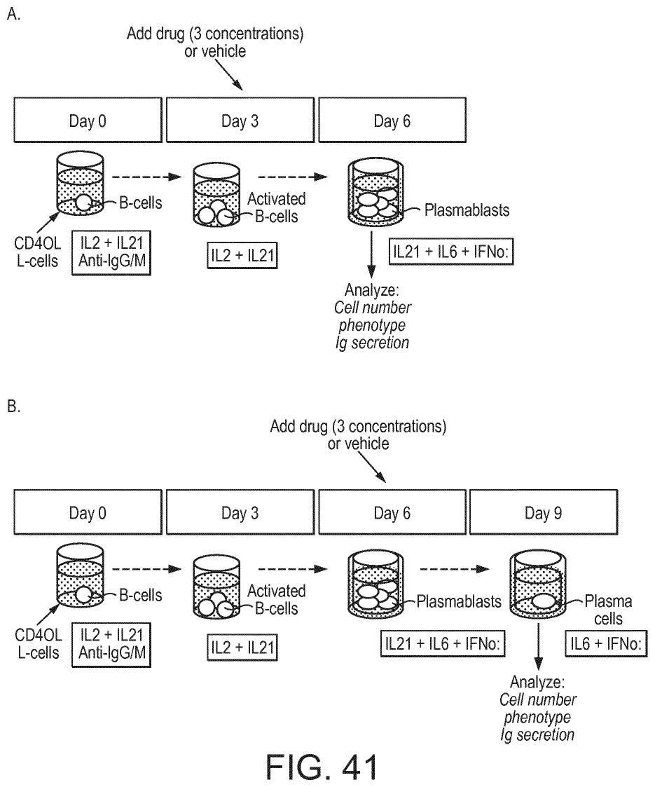

The system employed is based on a published model (Cocco et al., 2012) which uses a CD40L/IL-2/IL-21 based stimulus to drive B-cell activation and differentiation in a 3-step process to generate plasmablasts and functional polyclonal mature plasma cells (See FIG. 41). The final step of the culture (Day 6-9) was performed in the context of IFN-α driven survival signals and without stromal cells.

The experiment was performed using total peripheral blood B-cells isolated from healthy donors. The experiment was performed from four independent donors.

### Drug Addition:

Compounds were sourced from Tocris and dissolved in DMSO at the following concentrations:

### Clozapine:

350 ng/ml Clozapine (approximately equivalent to 500 mg adult human dose)
100 ng/ml Clozapine
25 ng/ml Clozapine (approximately equivalent to 55 mg adult human dose)

### Norclozapine:

200 ng/ml norclozapine
70 ng/ml norclozapine
15 ng/ml norclozapine

### Haloperidol:

25 ng/ml Haloperidol
8 ng/ml Haloperidol
2 ng/ml Haloperidol

DMSO as diluent control at 0.1%. All DMSO concentrations were adjusted to 0.1% for all drug treated samples.

Drugs were added at two time points:
day-3 of the culture (activated B-cell/pre-plasmablast), or
day-6 of the culture (plasmablast)

### Evaluation:

The cultures were evaluated 3 days after addition of the compound with day-3 drug additions evaluated at day-6 (plasmablast) and day-6 drug additions evaluated at day-9 (early plasma cell) (see FIG. 41).

### Evaluation Encompassed:

Flow cytometric assessment of:
phenotype (CD19, CD20, CD27, CD38, CD138)
viability (7AAD)
cell number (bead count)

### Immunoglobulin Secretion:

ELISA analysis of total IgM/IgG from bulk supernatant collected at day 6 and day 9 of respective cultures

### Results

### Cell Phenotype:

Across all four donors the control DMSO samples demonstrated a transition to a plasmablast state from day 3 to day 6 with downregulation of CD20, upregulation of CD38 and variable upregulation of CD27 combined with retained CD19 expression and lack of CD138. On subsequent transfer into plasma cell maturation conditions the control cells showed progressive loss of CD20, downregulation of CD19 and upregulation of CD138 combined with further upregulation of CD38 and CD27 indicating transition to early plasma cell state. These findings indicate that the differentiation protocol worked in relation to phenotype and that all four samples were suitable as references for the in vitro differentiation system.

In terms of effects on phenotypic maturation none of the drugs at any concentration showed significant effects on the downregulation of the B cell phenotype as reflected in equivalent loss of CD20 and CD19 expression. None of the drugs at any concentration showed significant effects on the pattern of acquisition of C27 or CD138 expression at either day 6 or day 9 time points.

All three drugs showed a dose related effect on the expression of CD38 in one donor. This was modest at the day 6 time point but was significant at the day 9 time point with a substantial and reproducible shift in CD38 expression. However, this effect was not observed as a consistent effect across the other donors.

### Cell Number and Viability:

Across all four donors the control DMSO samples demonstrated an expansion to the plasmablast state from day 3 to day 6 and contraction during the transition to plasma cell state. Based on an input activated B cell number at day 3 of 10⁵ the average expansion observed during the day 3 to day 6 culture was 12-fold. There was a 5-fold contraction that accompanied the maturation to the plasma cell state from 5×10⁵ input at day 6 to 10⁵ viable cells at day 9 was also consistent with past experience. It was concluded that the differentiation protocol worked as expected in relation to cell number and that all four samples are suitable as references.

None of the drugs at any concentration impacted significantly on the number of viable cells at either day 6 or day 9. This was not affected whether considering total cell number or viable cell number per input cell. Based on equivalent input activated B cell number the degree of expansion from day 3 to day 6 was equivalent across all drugs and concentrations. Equally there was no effect on the viable cell number recovered at day 9 with any drug at any concentration.

### Immunoglobulin Secretion:

Across all four donors the control DMSO samples showed evidence of significant IgM and IgG secretion at across the day 3 to day 6 culture. This was continued into the day 6 to day 9 culture with predicted higher per cell estimated secretion rates in this second culture phase to the plasma cell stated. It was concluded that the differentiation protocol worked in relation to immunoglobulin secretion and that all four samples are suitable as references.

In terms of immunoglobulin secretion there is greater variation between individual donors, but there were no clear trends in response to any of the three drugs at any dose. Normalising to DMSO as control provided the simplest view of the data and showed only minor shifts in the detected immunoglobulin in relation to IgG. Where changes are observed these follow inverse responses in relation to the dose for example norclozapine with one donor.

### Conclusion

The results showed that none of the drugs are directly toxic to differentiating B-cells, nor do any of the drugs at any concentration show consistent effects on the ability of the resulting differentiated antibody secreting cells to secrete antibody.

In terms of phenotypic responses there is variability between the donors in relation to CD38 expression with one donor in particular showing an apparent dose dependent downmodulation in the window of differentiation between plasmablast (day 6) and early plasma cell (day 9). However this response did not reproduce as a consistent feature across the other donors tested.

Overall, therefore, the compounds as tested do not show a consistent inhibitory effect on the functional or phenotypic maturation of activated B-cells to the early plasma cell state and have no effect on viability of antibody secreting cells.

The in vitro system employed has limitations in terms of being a ‘forced’ B cell differentiation assay (as opposed to physiological expansion), with a focus on peripheral B cells, limited culture duration which may not reflect effects of very chronic exposure, and lack of the normal micro-environment of B cells in primary (e.g. bone marrow) or secondary lymphoid tissues, nor indirect regulation (e.g. through T follicular helper cells and/or IL-21). Notwithstanding these, the findings suggest that clozapine is unlikely to be acting directly on plasma cells or their precursors and that the immunophenotypic findings in vivo reflect a more complex and/or indirect action. The findings from this in vitro study are consistent with the lack of reduction in overall B cell numbers (i.e. no evidence of generalized B cell depletion in patients taking clozapine).

### Summary of Results Set Out in Examples 1-5

The results set out in the examples above, encompassing observational data in humans treated with clozapine for prolonged periods of time, to short term dosing in healthy wild type mice in an immunologically unchallenged setting, to evaluation in a disease model of autoimmune disease with a major B cell component driven by antigen (CIA model), highlight several key effects of clozapine:

1. Reduction in total circulating immunoglobulin levels affecting all classes evaluated (IgG, IgM and IgA). While exhibiting interindividual variation, clozapine is seen to result in a leftward shift in the frequency distribution curve for these immunoglobulins. The robustness of this finding is highlighted by the interim findings in an orthogonal cohort of patients taking clozapine or other antipsychotics.
2. A relatively greater impact in human to reduce IgA (and IgM) compared to IgG, in part recapitulated with short-term dosing of wild type mice.
3. Evidence of progressive immunoglobulin (IgG) reduction with increasing duration of clozapine exposure in human. Conversely, evidence of gradual recovery (over years) of IgG on clozapine cessation.
4. Reduction in specific immunoglobulin. Beyond reductions in total immunoglobulin titre, clozapine is seen to lower pathogenic immunoglobulin (CIA model) and has been demonstrated by the inventors to lower pneumococcal specific antibody in human (Ponsford et al., 2018a), with the latter demonstrating a strong trend to significantly lower values on even interim analysis of the second observational cohort.
5. No significant impact on overall circulating (CD19+) B cells numbers. This observation contrasts sharply with the impact of current aggressive generalised B cell depleting biological approaches.
6. Substantial reductions in circulating plasmablasts (short-lived proliferating antibody secreting cells of the B cell lineage) and class-switched memory B cells. Both cell types are critical in the immediate and secondary humoral response. Class-switching enables a B cell to switch from IgM to production of the secondary IgH isotype antibodies IgG, IgA or IgE with different effector functions (Chaudhuri and Alt, 2004). Increased class-switching and plasma cell differentiation is recognised as a key feature in autoimmune disease associated with pathogenic immunoglobulin production (Suurmond et al., 2018). An ability of clozapine to inhibit this process, i.e. reduce class-switched memory B cells, suggests particular therapeutic potential in the setting of pathogenic immunoglobulin-mediated disorders which are primarily mediated by autoantibodies of the IgG, IgA or IgE subclass.
7. Subtle effects on bone marrow B cell precursors, specifically including a reduction in total pre B cells, proliferating pre B cells and immature B cells. This is notable for being a key endogenous transition checkpoint of B cell development for autoreactivity (Melchers, 2015). Defective B cell tolerance, including early tolerance, is recognised as a fundamental feature predisposing to autoimmunity (Samuels et al., 2005a; Yurasov et al., 2005). Accordingly, while speculative, it is possible that this effect of clozapine will serve to reduce further progression of B cells with autoreactivity (of the IgH chain) to modulate the emerging B cell repertoire.
8. Reduction in bone marrow long-lived plasma cells, a key cell population responsible for driving persistent autoimmune disease through the production of pathogenic immunoglobulin and which is substantially refractory to existing therapeutics.
9. The ability to substantially delay the onset of an experimental model of autoimmune disease with a substantial B cell-driven and pathogenic autoantibody component.
10. Reduce the proportion of B cells in secondary lymphoid tissue which, based on the findings from clozapine administration to wild type mice, does not appear to specifically affect one of the major B cell subsets in these tissues (specifically follicular B cells or marginal zone B cells).
11. Promote a significant increase in the proportion of Foxp3⁺ regulatory T cells (Tregs) in secondary lymphoid tissue in conjunction with an increase in the expression of the Treg marker CD25 (IL-2 receptor α-chains). Tregs are a specialised CD4⁺ T cell subset with a major immunoregulatory role in promoting immune tolerance and actively suppressing autoimmunity. IL-2 signalling is critical to maintaining Treg homeostasis and CD25 has been proposed to be used by Tregs to capture IL-2, thereby limiting its provision to and stimulation of effector CD4⁺ T cells to promote the latter's apoptosis. Accordingly, higher cell surface expression intensity of CD25 may serve to promote immunosuppressive Treg function.
12. Disruption of germinal centre function through effects on its key cellular components: induction of a profound reduction in germinal centre B cells together with a reduction in their level of activation/functionality. Coupled with this, clozapine is found to reduce surface expression of key proteins regulating T follicular helper cell positioning and functionality (PD1 and CXCR5). Germinal centres are the sites of intense proliferation and somatic mutation to result in differentiation of antigen-activated B cells into high affinity memory B cells or plasma cells. Accordingly, this finding (following antigen injection in the CIA model) is consistent with an impact of clozapine on distal B cell lineage maturation/function and modulation of T cell support of these processes. The net effect of this is concordant with observations set out in the examples demonstrating reduced class switched memory B cells, reduced plasmablast and long-lived plasma cell formation in response to clozapine. Together these actions will tend to reduce pathogenic immunoglobulin production in the setting of B cell driven autoimmune disease, including those with a T cell component.
13. Based on an in vitro differentiation assay, the observed effects of clozapine appear unlikely to reflect a direct effect on antibody secreting cells.

Thus, clozapine appears to have profound influence in vivo on the pathways involved in B cell maturation and pathogenic antibody (particularly pathogenic IgG and IgA antibody) production particularly via an impact on germinal centre T cell-B cell interaction, functionality and key regulators, likely potentiated by a reciprocal potentiation of immunosuppressive Foxp3⁺ Treg function. Clozapine is useful in treating pathogenic immunoglobulin driven B cell mediated diseases with a T cell component.

### Example 6

### Healthy Human Volunteer Study

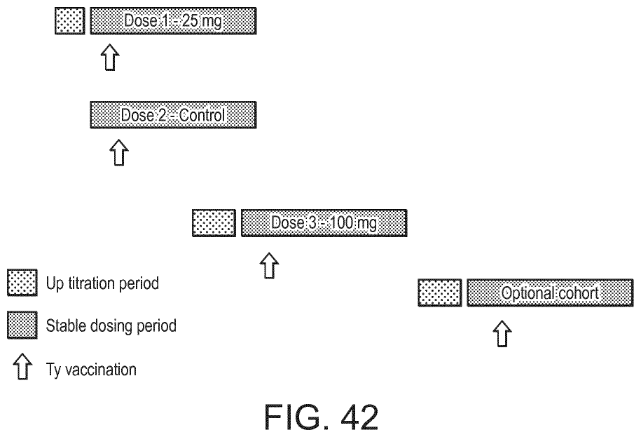

This study is a randomized unblinded controlled trial investigating the effects of low-dose clozapine on B cell number and function in healthy volunteers following vaccination (i.e. antigenic challenge). The study employs a parallel arm design (see FIG. 42) with a delayed start for the higher dose tested. In this study a total of up to 48 healthy volunteers will be recruited in to up to 4 cohorts. All participants will be administered Typhi immunization to stimulate the production of specific immunoglobulin (specifically IgG) at day 1 (immunization day) and followed for a period of approximately 56 days. Cohort 1 (n=12 participants) will be administered 25 mg of clozapine for 28 days and followed up for a further 28 days, whilst cohort 2 (n=12 participants, which will be recruited in parallel with Cohort1) will not receive any clozapine but will undergo vaccination. Cohort 2 will be followed in the same manner as cohort 1. Cohort 3 (100 mg clozapine) will only be initiated after the data from the active clozapine treatment period in cohort 1 (day 28 of active treatment) is reviewed by a Safety Committee. There is the potential for an optional cohort of another 12 healthy volunteers to be started if the data warrants further evaluation of doses between 25 and 100 mg clozapine.

Participants in Cohorts 1 and 2 will remain in the trial for a total of 60 days excluding their initial screening visit. Participants in Cohort 3 will take part for a total of 70 days excluding their initial screening visit.

The duration of participation for participants in the optional cohort 4 will vary depending on the dose chosen, due to the titration period being altered accordingly, but excluding their initial screening visit participants will participate for a maximum of 63 days (if a 100 mg dose is selected).

### Objectives and Outcome Measures

| | | Time point(s) of |
|---|---|---|
| | | evaluation of this |
| | | outcome measure |
| Objectives | Outcome Measures | (if applicable) |
| Primary Objective | Difference in specific anti-Typhim Vi | 28 days after |
| To understand the effect of | IgG 28 days after vaccination | vaccination |
| clozapine on primary vaccination | | |
| response | | |
| Secondary Objectives | Change from baseline in total | 28 days after |
| To determine the effect of | immunoglobulin levels (IgG, IgM and | vaccination |
| clozapine on circulating | IgA subclasses) | |
| immunoglobulin levels | | |
| To determine the effect of | Plasmablast response at seven days | 7 days after |
| clozapine on circulating | post- vaccination | vaccination |
| plasmablast levels | | |
| Exploratory Objectives | | |
| To understand the exposure- | Concentration response analysis to | All available |
| response relationship of clozapine | each primary and secondary end point | timepoints |
| on B cell subsets and | | |
| immunoglobulins | | |
| Effect of clozapine on | The difference in changes of specific | 28 days after |
| transcription profiles of sorted | RNA expression pre-clozapine dosing | vaccination |
| immune cells pre- and post- | and 28 days after vaccination between | |
| therapy | clozapine and control cohorts | |

Similar Immune Biomarkers will be collected in the Healthy Volunteer study to those in the observational study (Example 2).

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

### REFERENCES

Adam, L., Zoldan, K., Hofmann, M., Schultheiss, M., Bettinger, D., Neumann-Haefelin, C., Thimme, R., and Boettler, T. (2018). Follicular T Helper Cell Signatures in Primary Biliary Cholangitis and Primary Sclerosing Cholangitis. Hepatology communications 2, 1051-1063.
Adamus, G. (2003). Autoantibody-induced apoptosis as a possible mechanism of autoimmune retinopathy. Autoimmunity reviews 2, 63-68.
Aggarwal, R., Loganathan, P., Koontz, D., Qi, Z., Reed, A. M., and Oddis, C. V. (2017). Cutaneous improvement in refractory adult and juvenile dermatomyositis after treatment with rituximab. Rheumatology (Oxford, England) 56, 247-254.
Aggarwal, R., Oddis, C. V., Goudeau, D., Koontz, D., Qi, Z., Reed, A. M., Ascherman, D. P., and Levesque, M. C. (2016). Autoantibody levels in myositis patients correlate with clinical response during B cell depletion with rituximab. Rheumatology (Oxford, England) 55, 991-999.
Akashi, T., Nagafuchi, S., Anzai, K., Kondo, S., Kitamura, D., Wakana, S., Ono, J., Kikuchi, M., Niho, Y., and Watanabe, T. (1997). Direct evidence for the contribution of B cells to the progression of insulitis and the development of diabetes in non-obese diabetic mice. International immunology 9, 1159-1164.
Albers, L. N., Zone, J. J., Stoff, B. K., and Feldman, R. J. (2017). Rituximab Treatment for Recalcitrant Dermatitis Herpetiformis. JAMA dermatology 153, 315-318.
Allen, C. D., Okada, T., and Cyster, J. G. (2007). Germinal-center organization and cellular dynamics. Immunity 27, 190-202.
Allen, J. L., Fore, M. S., Wooten, J., Roehrs, P. A., Bhuiya, N. S., Hoffert, T., Sharf, A., Deal, A. M., Armistead, P., Coghill, J., et al. (2012). B cells from patients with chronic GVHD are activated and primed for survival via BAFF-mediated pathways. Blood 120, 2529-2536.
Alsaleh, G., Francois, A., Knapp, A. M., Schickel, J. N., Sibilia, J., Pasquali, J. L., Gottenberg, J. E., Wachsmann, D., and Soulas-Sprauel, P. (2011). Synovial fibroblasts promote immunoglobulin class switching by a mechanism involving BAFF. European journal of immunology 41, 2113-2122.
Amer, R., Bamonte, G., and Forrester, J. V. (2007). Resolution of juvenile idiopathic arthritis-associated uveitis after development of common variable immunodeficiency. European journal of ophthalmology 17, 666-668.
Andrews, B. S., Schenk, A., Barr, R., Friou, G., Mirick, G., and Ross, P. (1986). Immunopathology of cutaneous human lupus erythematosus defined by murine monoclonal antibodies. Journal of the American Academy of Dermatology 15, 474-481.
Arnold, D. M., Vrbensky, J. R., Karim, N., Smith, J. W., Liu, Y., Ivetic, N., Kelton, J. G., and Nazy, I. (2017). The effect of rituximab on anti-platelet autoantibody levels in patients with immune thrombocytopenia. British journal of haematology 178, 302-307.
Audia, S., Rossato, M., Santegoets, K., Spijkers, S., Wichers, C., Bekker, C., Bloem, A., Boon, L., Flinsenberg, T., Compeer, E., et al. (2014). Splenic TFH expansion participates in B-cell differentiation and antiplatelet-antibody production during immune thrombocytopenia. Blood 124, 2858-2866.
Audia, S., Samson, M., Guy, J., Janikashvili, N., Fraszczak, J., Trad, M., Ciudad, M., Leguy, V., Berthier, S., Petrella, T., et al. (2011). Immunologic effects of rituximab on the human spleen in immune thrombocytopenia. Blood 118, 4394-4400.
Avery, D. T., Bryant, V. L., Ma, C. S., de Waal Malefyt, R., and Tangye, S. G. (2008). IL-21-induced isotype switching to IgG and IgA by human naive B cells is differentially regulated by IL-4. Journal of immunology (Baltimore, Md.: 1950) 181, 1767-1779.
Baek, A., Park, H. J., Na, S. J., Shim, D. S., Moon, J. S., Yang, Y., and Choi, Y. C. (2012). The expression of BAFF in the muscles of patients with dermatomyositis. Journal of neuroimmunology 249, 96-100.
Baglaenko, Y., Chang, N. H., Johnson, S. R., Hafiz, W., Manion, K., Ferri, D., Noamani, B., Bonilla, D., Rusta-Sellehy, S., Lisnevskaia, L., et al. (2018). The presence of anti-nuclear antibodies alone is associated with changes in B cell activation and T follicular helper cells similar to those in systemic autoimmune rheumatic disease. Arthritis research & therapy 20, 264.
Baker, D., Marta, M., Pryce, G., Giovannoni, G., and Schmierer, K. (2017). Memory B Cells are Major Targets for Effective Immunotherapy in Relapsing Multiple Sclerosis. EBioMedicine 16, 41-50.
Banchereau, R., Hong, S., Cantarel, B., Baldwin, N., Baisch, J., Edens, M., Cepika, A. M., Acs, P., Turner, J., Anguiano, E., et al. (2016). Personalized Immunomonitoring Uncovers Molecular Networks that Stratify Lupus Patients. Cell 165, 551-565.
Becker, M. O., Kill, A., Kutsche, M., Guenther, J., Rose, A., Tabeling, C., Witzenrath, M., Kuhl, A. A., Heidecke, H., Ghofrani, H. A., et al. (2014). Vascular receptor autoantibodies in pulmonary arterial hypertension associated with systemic sclerosis. American journal of respiratory and critical care medicine 190, 808-817.
Belnoue, E., Pihlgren, M., McGaha, T. L., Tougne, C., Rochat, A. F., Bossen, C., Schneider, P., Huard, B., Lambert, P. H., and Siegrist, C. A. (2008). APRIL is critical for plasmablast survival in the bone marrow and poorly expressed by early-life bone marrow stromal cells. Blood 111, 2755-2764.
Ben-Skowronek, I., Szewczyk, L., Kulik-Rechberger, B., and Korobowicz, E. (2013). The differences in T and B cell subsets in thyroid of children with Graves' disease and Hashimoto's thyroiditis. World journal of pediatrics: WJP 9, 245-250.
Benner, R., Hijmans, W., and Haaijman, J. J. (1981). The bone marrow: the major source of serum immunoglobulins, but still a neglected site of antibody formation. Clinical and experimental immunology 46, 1-8.
Berggren, O., Hagberg, N., Alexsson, A., Weber, G., Ronnblom, L., and Eloranta, M. L. (2017). Plasmacytoid dendritic cells and RNA-containing immune complexes drive expansion of peripheral B cell subsets with an SLE-like phenotype. PloS one 12, e0183946.
Berglin, L., Bjorkstrom, N. K., and Bergquist, A. (2013). Primary sclerosing cholangitis is associated with autoreactive IgA antibodies against biliary epithelial cells. Scandinavian journal of gastroenterology 48, 719-728.
Bergqvist, P., Stensson, A., Lycke, N. Y., and Bemark, M. (2010). T cell-independent IgA class switch recombination is restricted to the GALT and occurs prior to manifest germinal center formation. Journal of immunology (Baltimore, Md.: 1950) 184, 3545-3553.
Bermejo, D. A., Jackson, S. W., Gorosito-Serran, M., Acosta-Rodriguez, E. V., Amezcua-Vesely, M. C., Sather, B. D., Singh, A. K., Khim, S., Mucci, J., Liggitt, D., et al. (2013). Trypanosoma cruzi trans-sialidase initiates a program independent of the transcription factors RORgammat and Ahr that leads to IL-17 production by activated B cells. Nature immunology 14, 514-522.
Bessis, N., Decker, P., Assier, E., Semerano, L., and Boissier, M. C. (2017). Arthritis models: usefulness and interpretation. Seminars in immunopathology 39, 469-486.
Betterle, C., Volpato, M., Rees Smith, B., Furmaniak, J., Chen, S., Greggio, N. A., Sanzari, M., Tedesco, F., Pedini, B., Boscaro, M., et al. (1997). I. Adrenal cortex and steroid 21-hydroxylase autoantibodies in adult patients with organ-specific autoimmune diseases: markers of low progression to clinical Addison's disease. The Journal of clinical endocrinology and metabolism 82, 932-938.
Bloise, F. F., Oliveira, F. L., Nobrega, A. F., Vasconcellos, R., Cordeiro, A., Paiva, L. S., Taub, D. D., Borojevic, R., Pazos-Moura, C. C., and Mello-Coelho, V. (2014). High levels of circulating triiodothyronine induce plasma cell differentiation. The Journal of endocrinology 220, 305-317.
Bonegio, R. G., Lin, J. D., Beaudette-Zlatanova, B., York, M. R., Menn-Josephy, H., and Yasuda, K. (2019). Lupus-Associated Immune Complexes Activate Human Neutrophils in an FcgammaRIIA-Dependent but TLR-Independent Response. Journal of immunology (Baltimore, Md.: 1950) 202, 675-683.
Bosello, S., Youinou, P., Daridon, C., Tolusso, B., Bendaoud, B., Pietrapertosa, D., Morelli, A., and Ferraccioli, G. (2008). Concentrations of BAFF correlate with autoantibody levels, clinical disease activity, and response to treatment in early rheumatoid arthritis. The Journal of rheumatology 35, 1256-1264.
Bradshaw, E. M., Orihuela, A., McArdel, S. L., Salajegheh, M., Amato, A. A., Hafler, D. A., Greenberg, S. A., and O'Connor, K. C. (2007). A local antigen-driven humoral response is present in the inflammatory myopathies. Journal of immunology (Baltimore, Md.: 1950) 178, 547-556.
Brand, D. D., Kang, A. H., and Rosloniec, E. F. (2004). The mouse model of collagen-induced arthritis. Methods in molecular medicine 102, 295-312.
Brand, D. D., Latham, K. A., and Rosloniec, E. F. (2007). Collagen-induced arthritis. Nature protocols 2, 1269-1275.
Brandtzaeg, P., Carlsen, H. S., and Halstensen, T. S. (2006). The B-cell system in inflammatory bowel disease. Advances in experimental medicine and biology 579, 149-167.
Bratland, E., and Husebye, E. S. (2011). Cellular immunity and immunopathology in autoimmune Addison's disease. Molecular and cellular endocrinology 336, 180-190.
Brito-Zeron, P., Baldini, C., Bootsma, H., Bowman, S. J., Jonsson, R., Mariette, X., Sivils, K., Theander, E., Tzioufas, A., and Ramos-Casals, M. (2016). Sjogren syndrome. Nature reviews Disease primers 2, 16047.
Bubier, J. A., Sproule, T. J., Foreman, O., Spolski, R., Shaffer, D. J., Morse, H. C., 3rd, Leonard, W. J., and Roopenian, D. C. (2009). A critical role for IL-21 receptor signaling in the pathogenesis of systemic lupus erythematosus in BXSB-Yaa mice. Proceedings of the National Academy of Sciences of the United States of America 106, 1518-1523.
Buckner, C. M., Moir, S., Kardava, L., Ho, J., Santich, B. H., Kim, L. J., Funk, E. K., Nelson, A. K., Winckler, B., Chairez, C. L., et al. (2014). CXCR4/IgG-expressing plasma cells are associated with human gastrointestinal tissue inflammation. The Journal of allergy and clinical immunology 133, 1676-1685.e1675.
Budeus, B., Schweigle de Reynoso, S., Przekopowitz, M., Hoffmann, D., Seifert, M., and Kuppers, R. (2015). Complexity of the human memory B-cell compartment is determined by the versatility of clonal diversification in germinal centers. Proceedings of the National Academy of Sciences of the United States of America 112, E5281-5289.
Burton, P. R., Clayton, D. G., Cardon, L. R., Craddock, N., Deloukas, P., Duncanson, A., Kwiatkowski, D. P., McCarthy, M. I., Ouwehand, W. H., Samani, N. J., et al. (2007). Association scan of 14,500 nonsynonymous SNPs in four diseases identifies autoimmunity variants. Nature genetics 39, 1329-1337.
Caja, S., Maki, M., Kaukinen, K., and Lindfors, K. (2011). Antibodies in celiac disease: implications beyond diagnostics. Cellular & molecular immunology 8, 103-109.
Cambridge, G., Leandro, M. J., Edwards, J. C., Ehrenstein, M. R., Salden, M., Bodman-Smith, M., and Webster, A. D. (2003). Serologic changes following B lymphocyte depletion therapy for rheumatoid arthritis. Arthritis and rheumatism 48, 2146-2154.
Campbell, D. A., Poulter, L. W., Janossy, G., and du Bois, R. M. (1985). Immunohistological analysis of lung tissue from patients with cryptogenic fibrosing alveolitis suggesting local expression of immune hypersensitivity. Thorax 40, 405-411.
Canete, J. D., Santiago, B., Cantaert, T., Sanmarti, R., Palacin, A., Celis, R., Graell, E., Gil-Torregrosa, B., Baeten, D., and Pablos, J. L. (2007). Ectopic lymphoid neogenesis in psoriatic arthritis. Annals of the rheumatic diseases 66, 720-726.
Cannons, J. L., Qi, H., Lu, K. T., Dutta, M., Gomez-Rodriguez, J., Cheng, J., Wakeland, E. K., Germain, R. N., and Schwartzberg, P. L. (2010). Optimal germinal center responses require a multistage T cell:B cell adhesion process involving integrins, SLAM-associated protein, and CD84. Immunity 32, 253-265.
Care, M. A., Stephenson, S. J., Barnes, N. A., Fan, I., Zougman, A., El-Sherbiny, Y. M., Vital, E. M., Westhead, D. R., Tooze, R. M., and Doody, G. M. (2016). Network Analysis Identifies Proinflammatory Plasma Cell Polarization for Secretion of ISG15 in Human Autoimmunity. Journal of immunology (Baltimore, Md.: 1950) 197, 1447-1459.
Carey, E. J., Ali, A. H., and Lindor, K. D. (2015). Primary biliary cirrhosis. Lancet (London, England) 386, 1565-1575.
Casola, S., Otipoby, K. L., Alimzhanov, M., Humme, S., Uyttersprot, N., Kutok, J. L., Carroll, M. C., and Rajewsky, K. (2004). B cell receptor signal strength determines B cell fate. Nature immunology 5, 317-327.
Cepok, S., Rosche, B., Grummel, V., Vogel, F., Zhou, D., Sayn, J., Sommer, N., Hartung, H. P., and Hemmer, B. (2005). Short-lived plasma blasts are the main B cell effector subset during the course of multiple sclerosis. Brain: a journal of neurology 128, 1667-1676.
Cervenak, L., Magyar, A., Boja, R., and Laszlo, G. (2001). Differential expression of GL7 activation antigen on bone marrow B cell subpopulations and peripheral B cells. Immunology letters 78, 89-96.
Chaudhuri, J., and Alt, F. W. (2004). Class-switch recombination: interplay of transcription, DNA deamination and DNA repair. Nature reviews Immunology 4, 541-552.
Chavele, K. M., Merry, E., and Ehrenstein, M. R. (2015). Cutting edge: circulating plasmablasts induce the differentiation of human T follicular helper cells via IL-6 production. Journal of immunology (Baltimore, Md.: 1950) 194, 2482-2485.
Chen, X., Ma, W., Zhang, T., Wu, L., and Qi, H. (2015). Phenotypic Tfh development promoted by CXCR5-controlled re-localization and IL-6 from radiation-resistant cells. Protein & cell 6, 825-832.
Chihara, N., Aranami, T., Oki, S., Matsuoka, T., Nakamura, M., Kishida, H., Yokoyama, K., Kuroiwa, Y., Hattori, N., Okamoto, T., et al. (2013). Plasmablasts as migratory IgG-producing cells in the pathogenesis of neuromyelitis optica. PloS one 8, e83036.
Chihara, N., Aranami, T., Sato, W., Miyazaki, Y., Miyake, S., Okamoto, T., Ogawa, M., Toda, T., and Yamamura, T. (2011). Interleukin 6 signaling promotes anti-aquaporin 4 autoantibody production from plasmablasts in neuromyelitis optica. Proceedings of the National Academy of Sciences of the United States of America 108, 3701-3706.
Chimen, M., McGettrick, H. M., Apta, B., Kuravi, S. J., Yates, C. M., Kennedy, A., Odedra, A., Alassiri, M., Harrison, M., Martin, A., et al. (2015). Homeostatic regulation of T cell trafficking by a B cell-derived peptide is impaired in autoimmune and chronic inflammatory disease. Nature medicine 21, 467-475.
Choi, Y. S., Kageyama, R., Eto, D., Escobar, T. C., Johnston, R. J., Monticelli, L., Lao, C., and Crotty, S. (2011). ICOS receptor instructs T follicular helper cell versus effector cell differentiation via induction of the transcriptional repressor Bcl6. Immunity 34, 932-946.
Chu, V. T., and Berek, C. (2013). The establishment of the plasma cell survival niche in the bone marrow. Immunological reviews 251, 177-188.
Chu, V. T., Frohlich, A., Steinhauser, G., Scheel, T., Roch, T., Fillatreau, S., Lee, J. J., Lohning, M., and Berek, C. (2011a). Eosinophils are required for the maintenance of plasma cells in the bone marrow. Nature immunology 12, 151-159.
Chu, X., Pan, C. M., Zhao, S. X., Liang, J., Gao, G. Q., Zhang, X. M., Yuan, G. Y., Li, C. G., Xue, L. Q., Shen, M., et al. (2011b). A genome-wide association study identifies two new risk loci for Graves' disease. Nature genetics 43, 897-901.
Chu, Y., Wang, F., Zhou, M., Chen, L., and Lu, Y. (2014). A preliminary study on the characterization of follicular helper T (Tfh) cells in rheumatoid arthritis synovium. Acta histochemica 116, 539-543.
Chua, I., Lagos, M., Charalambous, B. M., Workman, S., Chee, R., and Grimbacher, B. (2011). Pathogen-specific IgG antibody levels in immunodeficient patients receiving immunoglobulin replacement do not provide additional benefit to therapeutic management over total serum IgG. The Journal of allergy and clinical immunology 127, 1410-1411.
Chung, B. K., Guevel, B. T., Reynolds, G. M., Gupta Udatha, D. B., Henriksen, E. K., Stamataki, Z., Hirschfield, G. M., Karlsen, T. H., and Liaskou, E. (2017). Phenotyping and auto-antibody production by liver-infiltrating B cells in primary sclerosing cholangitis and primary biliary cholangitis. Journal of autoimmunity 77, 45-54.
Chung, B. K., Henriksen, E. K. K., Jorgensen, K. K., Karlsen, T. H., Hirschfield, G. M., and Liaskou, E. (2018). Gut and Liver B Cells of Common Clonal Origin in Primary Sclerosing Cholangitis-Inflammatory Bowel Disease. Hepatology communications 2, 956-967.
Chung, B. K., Udatha, G., Pharo, A., Taussig, M., Fonseca, C., Stoevesandt, O., Mehta, A., Lund-Johansen, F., Hirschfield, G. M., Karlsen, T. H., et al. (2016). Liver-Infiltrating B Cells Produce Autoantibodies that Point toward Novel Autoantigens in Primary Sclerosing Cholangitis. Journal of hepatology 64, S433.
Chung, Y., Tanaka, S., Chu, F., Nurieva, R. I., Martinez, G. J., Rawal, S., Wang, Y. H., Lim, H., Reynolds, J. M., Zhou, X. H., et al. (2011). Follicular regulatory T cells expressing Foxp3 and Bcl-6 suppress germinal center reactions. Nature medicine 17, 983-988.
Clavel, C., Nogueira, L., Laurent, L., Iobagiu, C., Vincent, C., Sebbag, M., and Serre, G. (2008). Induction of macrophage secretion of tumor necrosis factor alpha through Fcgamma receptor IIa engagement by rheumatoid arthritis-specific autoantibodies to citrullinated proteins complexed with fibrinogen. Arthritis and rheumatism 58, 678-688.
Cocco, M., Stephenson, S., Care, M. A., Newton, D., Barnes, N. A., Davison, A., Rawstron, A., Westhead, D. R., Doody, G. M., and Tooze, R. M. (2012). In vitro generation of long-lived human plasma cells. Journal of immunology (Baltimore, Md.: 1950) 189, 5773-5785.
Cohen, S. B., Emery, P., Greenwald, M. W., Dougados, M., Furie, R. A., Genovese, M. C., Keystone, E. C., Loveless, J. E., Burmester, G. R., Cravets, M. W., et al. (2006). Rituximab for rheumatoid arthritis refractory to anti-tumor necrosis factor therapy: Results of a multicenter, randomized, double-blind, placebo-controlled, phase III trial evaluating primary efficacy and safety at twenty-four weeks. Arthritis and rheumatism 54, 2793-2806.
Collin, P., Salmi, T. T., Hervonen, K., Kaukinen, K., and Reunala, T. (2017). Dermatitis herpetiformis: a cutaneous manifestation of coeliac disease. Annals of medicine 49, 23-31.
Colucci, R., Lotti, F., Dragoni, F., Arunachalam, M., Lotti, T., Benvenga, S., and Moretti, S. (2014). High prevalence of circulating autoantibodies against thyroid hormones in vitiligo and correlation with clinical and historical parameters of patients. The British journal of dermatology 171, 786-798.
Conrad, K., Roggenbuck, D., and Laass, M. W. (2014). Diagnosis and classification of ulcerative colitis. Autoimmunity reviews 13, 463-466.
Craig, S. W., and Cebra, J. J. (1971). Peyer's patches: an enriched source of precursors for IgA-producing immunocytes in the rabbit. The Journal of experimental medicine 134, 188-200.
Cross, A. H., Stark, J. L., Lauber, J., Ramsbottom, M. J., and Lyons, J. A. (2006). Rituximab reduces B cells and T cells in cerebrospinal fluid of multiple sclerosis patients. Journal of neuroimmunology 180, 63-70.
Crotty, S. (2011). Follicular helper CD4 T cells (TFH). Annual review of immunology 29, 621-663.
Cui, J., Arita, Y., and Bystryn, J. C. (1993). Cytolytic antibodies to melanocytes in vitiligo. The Journal of investigative dermatology 100, 812-815.
Cui, J., Harning, R., Henn, M., and Bystryn, J. C. (1992). Identification of pigment cell antigens defined by vitiligo antibodies. The Journal of investigative dermatology 98, 162-165.
Cupi, M. L., Sarra, M., Marafini, I., Monteleone, I., Franze, E., Ortenzi, A., Colantoni, A., Sica, G., Sileri, P., Rosado, M. M., et al. (2014). Plasma cells in the mucosa of patients with inflammatory bowel disease produce granzyme B and possess cytotoxic activities. Journal of immunology (Baltimore, Md.: 1950) 192, 6083-6091.
Dandah, A., Habir, K., Nandakumar, K. S., Saxena, A., Xu, B., Holmdahl, R., and Malin, S. (2018). Germinal Center B Cells Are Essential for Collagen-Induced Arthritis. Arthritis & rheumatology (Hoboken, N.J.) 70, 193-203.
Dalakas, M. C. (2015). Inflammatory muscle diseases. The New England journal of medicine 372, 1734-1747.
Dalin, F., Nordling Eriksson, G., Dahlqvist, P., Hallgren, A., Wahlberg, J., Ekwall, O., Soderberg, S., Ronnelid, J., Olcen, P., Winqvist, O., et al. (2017). Clinical and Immunological Characteristics of Autoimmune Addison Disease: A Nationwide Swedish Multicenter Study. The Journal of clinical endocrinology and metabolism 102, 379-389.
Daoussis, D., Melissaropoulos, K., Sakellaropoulos, G., Antonopoulos, I., Markatseli, T. E., Simopoulou, T., Georgiou, P., Andonopoulos, A. P., Drosos, A. A., Sakkas, L., et al. (2017). A multicenter, open-label, comparative study of B-cell depletion therapy with Rituximab for systemic sclerosis-associated interstitial lung disease. Seminars in arthritis and rheumatism 46, 625-631.
Dass, S., Vital, E. M., and Emery, P. (2007). Development of psoriasis after B cell depletion with rituximab. Arthritis and rheumatism 56, 2715-2718.
Davies, R. J., Sangle, S. R., Jordan, N. P., Aslam, L., Lewis, M. J., Wedgwood, R., and D'Cruz, D. P. (2013). Rituximab in the treatment of resistant lupus nephritis: therapy failure in rapidly progressive crescentic lupus nephritis. Lupus 22, 574-582.
Dawoodji, A., Chen, J. L., Shepherd, D., Dalin, F., Tarlton, A., Alimohammadi, M., Penna-Martinez, M., Meyer, G., Mitchell, A. L., Gan, E. H., et al. (2014). High frequency of cytolytic 21-hydroxylase-specific CD8+ T cells in autoimmune Addison's disease patients. Journal of immunology (Baltimore, Md.: 1950) 193, 2118-2126.
Dawson, L. J., Stanbury, J., Venn, N., Hasdimir, B., Rogers, S. N., and Smith, P. M. (2006). Antimuscarinic antibodies in primary Sjogren's syndrome reversibly inhibit the mechanism of fluid secretion by human submandibular salivary acinar cells. Arthritis and rheumatism 54, 1165-1173.
De Bellis, A. A., Falorni, A., Laureti, S., Perrino, S., Coronella, C., Forini, F., Bizzarro, E., Bizzarro, A., Abbate, G., and Bellastella, A. (2001). Time course of 21-hydroxylase antibodies and long-term remission of subclinical autoimmune adrenalitis after corticosteroid therapy: case report. The Journal of clinical endocrinology and metabolism 86, 675-678.
Denton, C. P., and Khanna, D. (2017). Systemic sclerosis. Lancet (London, England) 390, 1685-1699.
Derksen, V., Huizinga, T. W. J., and van der Woude, D. (2017). The role of autoantibodies in the pathophysiology of rheumatoid arthritis. Seminars in immunopathology 39, 437-446.
Dieterich, W., Ehnis, T., Bauer, M., Donner, P., Volta, U., Riecken, E. O., and Schuppan, D. (1997). Identification of tissue transglutaminase as the autoantigen of celiac disease. Nature medicine 3, 797-801.
Dobashi, N., Fujita, J., Murota, M., Ohtsuki, Y., Yamadori, I., Yoshinouchi, T., Ueda, R., Bandoh, S., Kamei, T., Nishioka, M., et al. (2000). Elevation of anti-cytokeratin 18 antibody and circulating cytokeratin 18: anti-cytokeratin 18 antibody immune complexes in sera of patients with idiopathic pulmonary fibrosis. Lung 178, 171-179.
Donahoe, M., Valentine, V. G., Chien, N., Gibson, K. F., Raval, J. S., Saul, M., Xue, J., Zhang, Y., and Duncan, S. R. (2015). Autoantibody-Targeted Treatments for Acute Exacerbations of Idiopathic Pulmonary Fibrosis. PloS one 10, e0127771.
Dong, W., Li, X., Liu, H., and Zhu, P. (2009). Infiltrations of plasma cells in synovium are highly associated with synovial fluid levels of APRIL in inflamed peripheral joints of rheumatoid arthritis. Rheumatology international 29, 801-806.
Dorfman, D. M., Brown, J. A., Shahsafaei, A., and Freeman, G. J. (2006). Programmed death-1 (PD-1) is a marker of germinal center-associated T cells and angioimmunoblastic T-cell lymphoma. The American journal of surgical pathology 30, 802-810.
du Pre, M. F., and Sollid, L. M. (2015). T-cell and B-cell immunity in celiac disease. Best practice & research Clinical gastroenterology 29, 413-423.
Duan, B., Niu, H., Xu, Z., Sharpe, A. H., Croker, B. P., Sobel, E. S., and Morel, L. (2008). Intrafollicular location of marginal zone/CD1d(hi) B cells is associated with autoimmune pathology in a mouse model of lupus. Laboratory investigation; a journal of technical methods and pathology 88, 1008-1020.
Durie, F. H., Fava, R. A., Foy, T. M., Aruffo, A., Ledbetter, J. A., and Noelle, R. J. (1993). Prevention of collagen-induced arthritis with an antibody to gp39, the ligand for CD40. Science (New York, N.Y.) 261, 1328-1330.
Ebbo, M., Grados, A., Samson, M., Groh, M., Loundou, A., Rigolet, A., Terrier, B., Guillaud, C., Carra-Dalliere, C., Renou, F., et al. (2017). Long-term efficacy and safety of rituximab in IgG4-related disease: Data from a French nationwide study of thirty-three patients. PloS one 12, e0183844.
Edwards, J. C., Szczepanski, L., Szechinski, J., Filipowicz-Sosnowska, A., Emery, P., Close, D. R., Stevens, R. M., and Shaw, T. (2004). Efficacy of B-cell-targeted therapy with rituximab in patients with rheumatoid arthritis. The New England journal of medicine 350, 2572-2581.
Eggers, E. L., Michel, B. A., Wu, H., Wang, S. Z., Bevan, C. J., Abounasr, A., Pierson, N. S., Bischof, A., Kazer, M., Leitner, E., et al. (2017). Clonal relationships of CSF B cells in treatment-naive multiple sclerosis patients. JCI insight 2.
Ehrenstein, M. R., Evans, J. G., Singh, A., Moore, S., Warnes, G., Isenberg, D. A., and Mauri, C. (2004). Compromised function of regulatory T cells in rheumatoid arthritis and reversal by anti-TNFalpha therapy. The Journal of experimental medicine 200, 277-285.
Elliott, C., Lindner, M., Arthur, A., Brennan, K., Jarius, S., Hussey, J., Chan, A., Stroet, A., Olsson, T., Willison, H., et al. (2012). Functional identification of pathogenic autoantibody responses in patients with multiple sclerosis. Brain: a journal of neurology 135, 1819-1833.
Endo, T., Ito, K., Morimoto, J., Kanayama, M., Ota, D., Ikesue, M., Kon, S., Takahashi, D., Onodera, T., Iwasaki, N., et al. (2015). Syndecan 4 Regulation of the Development of Autoimmune Arthritis in Mice by Modulating B Cell Migration and Germinal Center Formation. Arthritis & rheumatology (Hoboken, N.J.) 67, 2512-2522.
Eriksson, D., Bianchi, M., Landegren, N., Nordin, J., Dalin, F., Mathioudaki, A., Eriksson, G. N., Hultin-Rosenberg, L., Dahlqvist, J., Zetterqvist, H., et al. (2016). Extended exome sequencing identifies BACH2 as a novel major risk locus for Addison's disease. Journal of internal medicine 280, 595-608.
Ettinger, R., Sims, G. P., Fairhurst, A. M., Robbins, R., da Silva, Y. S., Spolski, R., Leonard, W. J., and
Lipsky, P. E. (2005). IL-21 induces differentiation of human naive and memory B cells into antibody-secreting plasma cells. Journal of immunology (Baltimore, Md.: 1950) 175, 7867-7879.
Ezzedine, K., Eleftheriadou, V., Whitton, M., and van Geel, N. (2015). Vitiligo. Lancet (London, England) 386, 74-84.
Famularo, G., Giacomelli, R., Alesse, E., Cifone, M. G., Morrone, S., Boirivant, M., Danese, C., Perego, M. A., Santoni, A., and Tonietti, G. (1989). Polyclonal B lymphocyte activation in progressive systemic sclerosis. Journal of clinical & laboratory immunology 29, 59-63.
Feghali-Bostwick, C. A., Tsai, C. G., Valentine, V. G., Kantrow, S., Stoner, M. W., Pilewski, J. M., Gadgil, A., George, M. P., Gibson, K. F., Choi, A. M., et al. (2007). Cellular and humoral autoreactivity in idiopathic pulmonary fibrosis. Journal of immunology (Baltimore, Md.: 1950) 179, 2592-2599.
Feng, X., Wang, D., Chen, J., Lu, L., Hua, B., Li, X., Tsao, B. P., and Sun, L. (2012). Inhibition of aberrant circulating Tfh cell proportions by corticosteroids in patients with systemic lupus erythematosus. PloS one 7, e51982.
Findlay, A. R., Goyal, N. A., and Mozaffar, T. (2015). An overview of polymyositis and dermatomyositis. Muscle & nerve 51, 638-656.
Firkin, B. G., Wright, R., Miller, S., and Stokes, E. (1969). Splenic macrophages in thrombocytopenia. Blood 33, 240-245.
Fisher, B. A., Everett, C. C., Rout, J., O'Dwyer, J. L., Emery, P., Pitzalis, C., Ng, W. F., Carr, A., Pease, C. T., Price, E. J., et al. (2018). Effect of rituximab on a salivary gland ultrasound score in primary Sjogren's syndrome: results of the TRACTISS randomised double-blind multicentre substudy. Annals of the rheumatic diseases 77, 412-416.
Fleischmajer, R., Perlish, J. S., and Reeves, J. R. (1977). Cellular infiltrates in scleroderma skin. Arthritis and rheumatism 20, 975-984.
Forestier, A., Guerrier, T., Jouvray, M., Giovannelli, J., Lefevre, G., Sobanski, V., Hauspie, C., Hachulla, E., Hatron, P. Y., Zephir, H., et al. (2018). Altered B lymphocyte homeostasis and functions in systemic sclerosis. Autoimmunity reviews 17, 244-255.
Foy, T. M., Laman, J. D., Ledbetter, J. A., Aruffo, A., Claassen, E., and Noelle, R. J. (1994). gp39-CD40 interactions are essential for germinal center formation and the development of B cell memory. The Journal of experimental medicine 180, 157-163.
Francois, A., Chatelus, E., Wachsmann, D., Sibilia, J., Bahram, S., Alsaleh, G., and Gottenberg, J. E. (2013). B lymphocytes and B-cell activating factor promote collagen and profibrotic markers expression by dermal fibroblasts in systemic sclerosis. Arthritis research & therapy 15, R168.
Fritz, J. H., Rojas, O. L., Simard, N., McCarthy, D. D., Hapfelmeier, S., Rubino, S., Robertson, S. J., Larijani, M., Gosselin, J., Ivanov, I I, et al. (2011). Acquisition of a multifunctional IgA⁺ plasma cell phenotype in the gut. Nature 481, 199-203.
Fuentes-Duculan, J., Bonifacio, K. M., Hawkes, J. E., Kunjravia, N., Cueto, I., Li, X., Gonzalez, J., Garcet, S., and Krueger, J. G. (2017). Autoantigens ADAMTSL5 and LL37 are significantly upregulated in active Psoriasis and localized with keratinocytes, dendritic cells and other leukocytes. Experimental dermatology 26, 1075-1082.
Furst, D., Felson, D., Thoren, G., and Gendreau, R. M. (2000). Immunoadsorption for the treatment of rheumatoid arthritis: final results of a randomized trial. Prosorba Trial Investigators. Therapeutic apheresis: official journal of the International Society for Apheresis and the Japanese Society for Apheresis 4, 363-373.
Gal, B., Dulic, S., Kiss, M., Groma, G., Kovacs, L., Kemeny, L., and Bata-Csorgo, Z. (2017). Increased circulating anti-alpha6-integrin autoantibodies in psoriasis and psoriatic arthritis but not in rheumatoid arthritis. The Journal of dermatology 44, 370-374.
Gheita, T. A., Bassyouni, I. H., Emad, Y., el-Din, A. M., Abdel-Rasheed, E., and Hussein, H. (2012). Elevated BAFF (BLyS) and APRIL in Juvenile idiopathic arthritis patients: relation to clinical manifestations and disease activity. Joint, bone, spine: revue du rhumatisme 79, 285-290.
Gidwaney, N. G., Pawa, S., and Das, K. M. (2017). Pathogenesis and clinical spectrum of primary sclerosing cholangitis. World journal of gastroenterology 23, 2459-2469.
Godfrey, W. A., Lindsley, C. B., and Cuppage, F. E. (1981). Localization of IgM in plasma cells in the iris of a patient with iridocyclitis and juvenile rheumatoid arthritis. Arthritis and rheumatism 24, 1195-1198.
Good-Jacobson, K. L., Szumilas, C. G., Chen, L., Sharpe, A. H., Tomayko, M. M., and Shlomchik, M. J. (2010). PD-1 regulates germinal center B cell survival and the formation and affinity of long-lived plasma cells. Nature immunology 11, 535-542.
Gottumukkala, R. V., Gavalas, N. G., Akhtar, S., Metcalfe, R. A., Gawkrodger, D. J., Haycock, J. W., Watson, P. F., Weetman, A. P., and Kemp, E. H. (2006). Function-blocking autoantibodies to the melanin-concentrating hormone receptor in vitiligo patients. Laboratory investigation; a journal of technical methods and pathology 86, 781-789.
Grange, L., Dalai, M., Nussenblatt, R. B., and Sen, H. N. (2014). Autoimmune retinopathy. American journal of ophthalmology 157, 266-272.e261.
Gray, L., van den Buuse, M., Scarr, E., Dean, B., and Hannan, A. J. (2009). Clozapine reverses schizophrenia-related behaviours in the metabotropic glutamate receptor 5 knockout mouse: association with N-methyl-D-aspartic acid receptor up-regulation. The international journal of neuropsychopharmacology 12, 45-60.
Greb, J. E., Goldminz, A. M., Elder, J. T., Lebwohl, M. G., Gladman, D. D., Wu, J. J., Mehta, N. N., Finlay, A. Y., and Gottlieb, A. B. (2016). Psoriasis. Nature reviews Disease primers 2, 16082.
Greeley, S. A., Katsumata, M., Yu, L., Eisenbarth, G. S., Moore, D. J., Goodarzi, H., Barker, C. F., Naji, A., and Noorchashm, H. (2002). Elimination of maternally transmitted autoantibodies prevents diabetes in nonobese diabetic mice. Nature medicine 8, 399-402.
Greenberg, S. A., Bradshaw, E. M., Pinkus, J. L., Pinkus, G. S., Burleson, T., Due, B., Bregoli, L., O'Connor, K. C., and Amato, A. A. (2005). Plasma cells in muscle in inclusion body myositis and polymyositis. Neurology 65, 1782-1787.
Greidinger, E. L., Zang, Y., Jaimes, K., Hogenmiller, S., Nassiri, M., Bejarano, P., Barber, G. N., and Hoffman, R. W. (2006). A murine model of mixed connective tissue disease induced with U1 small nuclear RNP autoantigen. Arthritis and rheumatism 54, 661-669.
Groom, J., Kalled, S. L., Cutler, A. H., Olson, C., Woodcock, S. A., Schneider, P., Tschopp, J., Cachero, T. G., Batten, M., Wheway, J., et al. (2002). Association of BAFF/BLyS overexpression and altered B cell differentiation with Sjogren's syndrome. The Journal of clinical investigation 109, 59-68.
Guitton, C., Kinowski, J. M., Abbar, M., Chabrand, P., and Bressolle, F. (1999). Clozapine and metabolite concentrations during treatment of patients with chronic schizophrenia. Journal of clinical pharmacology 39, 721-728.
Gunther, J., Rademacher, J., van Laar, J. M., Siegert, E., and Riemekasten, G. (2015). Functional autoantibodies in systemic sclerosis. Seminars in immunopathology 37, 529-542.
Hafkenscheid, L., Bondt, A., Scherer, H. U., Huizinga, T. W., Wuhrer, M., Toes, R. E., and Rombouts, Y. (2017). Structural Analysis of Variable Domain Glycosylation of Anti-Citrullinated Protein Antibodies in Rheumatoid Arthritis Reveals the Presence of Highly Sialylated Glycans. Molecular & cellular proteomics: MCP 16, 278-287.
Hagn, M., Ebel, V., Sontheimer, K., Schwesinger, E., Lunov, O., Beyer, T., Fabricius, D., Barth, T. F., Viardot, A., Stilgenbauer, S., et al. (2010). CD5+ B cells from individuals with systemic lupus erythematosus express granzyme B. European journal of immunology 40, 2060-2069.
Hajas, A., Barath, S., Szodoray, P., Nakken, B., Gogolak, P., Szekanecz, Z., Zold, E., Zeher, M., Szegedi, G., and Bodolay, E. (2013). Derailed B cell homeostasis in patients with mixed connective tissue disease. Human immunology 74, 833-841.
Halstensen, T. S., Hvatum, M., Scott, H., Fausa, O., and Brandtzaeg, P. (1992). Association of subepithelial deposition of activated complement and immunoglobulin G and M response to gluten in celiac disease. Gastroenterology 102, 751-759.
Halttunen, T., and Maki, M. (1999). Serum immunoglobulin A from patients with celiac disease inhibits human T84 intestinal crypt epithelial cell differentiation. Gastroenterology 116, 566-572.
Hamilton, A. L., Kamm, M. A., De Cruz, P., Wright, E. K., Selvaraj, F., Princen, F., Gorelik, A., Liew, D., Lawrance, I. C., Andrews, J. M., et al. (2017). Serologic antibodies in relation to outcome in postoperative Crohn's disease. Journal of gastroenterology and hepatology 32, 1195-1203.
Hammarlund, E., Thomas, A., Amanna, I. J., Holden, L. A., Slayden, O. D., Park, B., Gao, L., and Slifka, M. K. (2017). Plasma cell survival in the absence of B cell memory. Nature communications 8, 1781.
Handin, R. I., and Stossel, T. P. (1974). Phagocytosis of antibody-coated platelets by human granulocytes. The New England journal of medicine 290, 989-993.
Hansen, A., Lipsky, P. E., and Dorner, T. (2007). B cells in Sjogren's syndrome: indications for disturbed selection and differentiation in ectopic lymphoid tissue. Arthritis research & therapy 9, 218.
Hardy, S., Hashemi, K., Catanese, M., Candil, M., Zufferey, P., Gabison, E., and Guex-Crosier, Y. (2017). Necrotising Scleritis and Peripheral Ulcerative Keratitis Associated with Rheumatoid Arthritis Treated with Rituximab. Klinische Monatsblatter fur Augenheilkunde 234, 567-570.
Harrington, W. J., Minnich, V., Hollingsworth, J. W., and Moore, C. V. (1951). Demonstration of a thrombocytopenic factor in the blood of patients with thrombocytopenic purpura. The Journal of laboratory and clinical medicine 38, 1-10.
Hauser, S. L., Bar-Or, A., Comi, G., Giovannoni, G., Hartung, H. P., Hemmer, B., Lublin, F., Montalban, X., Rammohan, K. W., Selmaj, K., et al. (2017). Ocrelizumab versus Interferon Beta-1a in Relapsing Multiple Sclerosis. The New England journal of medicine 376, 221-234.
Hauser, S. L., Waubant, E., Arnold, D. L., Vollmer, T., Antel, J., Fox, R. J., Bar-Or, A., Panzara, M., Sarkar, N., Agarwal, S., et al. (2008). B-cell depletion with rituximab in relapsing-remitting multiple sclerosis. The New England journal of medicine 358, 676-688.
Hayashi, M., Yanaba, K., Umezawa, Y., Yoshihara, Y., Kikuchi, S., Ishiuji, Y., Saeki, H., and Nakagawa, H. (2016). IL-10-producing regulatory B cells are decreased in patients with psoriasis. Journal of dermatological science 81, 93-100.
He, R., Reid, D. M., Jones, C. E., and Shulman, N. R. (1994). Spectrum of Ig classes, specificities, and titers of serum antiglycoproteins in chronic idiopathic thrombocytopenic purpura. Blood 83, 1024-1032.
Hiepe, F., Dorner, T., Hauser, A. E., Hoyer, B. F., Mei, H., and Radbruch, A. (2011). Long-lived autoreactive plasma cells drive persistent autoimmune inflammation. Nature reviews Rheumatology 7, 170-178.
Hinze-Selch, D., Becker, E. W., Stein, G. M., Berg, P. A., Mullington, J., Holsboer, F., and Pollmacher, T. (1998). Effects of clozapine on in vitro immune parameters: a longitudinal study in clozapine-treated schizophrenic patients. Neuropsychopharmacology: official publication of the American College of Neuropsychopharmacology 19, 114-122.
Hofmann, S. C., Leandro, M. J., Morris, S. D., and Isenberg, D. A. (2013). Effects of rituximab-based B-cell depletion therapy on skin manifestations of lupus erythematosus—report of 17 cases and review of the literature. Lupus 22, 932-939.
Hov, J. R., Boberg, K. M., and Karlsen, T. H. (2008). Autoantibodies in primary sclerosing cholangitis. World journal of gastroenterology 14, 3781-3791.
Hoyer, B. F., Moser, K., Hauser, A. E., Peddinghaus, A., Voigt, C., Eilat, D., Radbruch, A., Hiepe, F., and Manz, R. A. (2004). Short-lived plasmablasts and long-lived plasma cells contribute to chronic humoral autoimmunity in NZB/W mice. The Journal of experimental medicine 199, 1577-1584.
Hu, C. Y., Rodriguez-Pinto, D., Du, W., Ahuja, A., Henegariu, O., Wong, F. S., Shlomchik, M. J., and Wen, L. (2007). Treatment with CD20-specific antibody prevents and reverses autoimmune diabetes in mice. The Journal of clinical investigation 117, 3857-3867.
Hull, C. M., Liddle, M., Hansen, N., Meyer, L. J., Schmidt, L., Taylor, T., Jaskowski, T. D., Hill, H. R., and Zone, J. J. (2008). Elevation of IgA anti-epidermal transglutaminase antibodies in dermatitis herpetiformis. The British journal of dermatology 159, 120-124.
Humby, F., Bombardieri, M., Manzo, A., Kelly, S., Blades, M. C., Kirkham, B., Spencer, J., and Pitzalis, C. (2009). Ectopic lymphoid structures support ongoing production of class-switched autoantibodies in rheumatoid synovium. PLoS medicine 6, el.
Hung, G. C., Liu, H. C., Yang, S. Y., Pan, C. H., Liao, Y. T., Chen, C. C., and Kuo, C. J. (2016). Antipsychotic reexposure and recurrent pneumonia in schizophrenia: a nested case-control study. The Journal of clinical psychiatry 77, 60-66.
Hussein, M. R., Aboulhagag, N. M., Atta, H. S., and Atta, S. M. (2008). Evaluation of the profile of the immune cell infiltrate in lichen planus, discoid lupus erythematosus, and chronic dermatitis. Pathology 40, 682-693.
Iaccarino, L., Bartoloni, E., Carli, L., Ceccarelli, F., Conti, F., De Vita, S., Ferraccioli, G., Galeazzi, M., Gatto, M., Gerli, R., et al. (2015). Efficacy and safety of off-label use of rituximab in refractory lupus: data from the Italian Multicentre Registry. Clinical and experimental rheumatology 33, 449-456.
Inoue, Y., Kaifu, T., Sugahara-Tobinai, A., Nakamura, A., Miyazaki, J., and Takai, T. (2007). Activating Fc gamma receptors participate in the development of autoimmune diabetes in NOD mice. Journal of immunology (Baltimore, Md.: 1950) 179, 764-774.
Ishisaka, M., Tsujii, S., Mizoguchi, T., Tsuruma, K., Shimazawa, M., and Hara, H. (2015). The effects of valproate and olanzapine on the abnormal behavior of diacylglycerol kinase beta knockout mice. Pharmacological reports: PR 67, 275-280.
Jacobi, A. M., Zhang, J., Mackay, M., Aranow, C., and Diamond, B. (2009). Phenotypic characterization of autoreactive B cells—checkpoints of B cell tolerance in patients with systemic lupus erythematosus. PloS one 4, e5776.
Jasiek, M., Karras, A., Le Guern, V., Krastinova, E., Mesbah, R., Faguer, S., Jourde-Chiche, N., Fauchais, A. L., Chiche, L., Dernis, E., et al. (2017). A multicentre study of 95 biopsy-proven cases of renal disease in primary Sjogren's syndrome. Rheumatology (Oxford, England) 56, 362-370.
Jelcic, I., Al Nimer, F., Wang, J., Lentsch, V., Planas, R., Jelcic, I., Madjovski, A., Ruhrmann, S., Faigle, W., Frauenknecht, K., et al. (2018). Memory B Cells Activate Brain-Homing, Autoreactive CD4(+) T Cells in Multiple Sclerosis. Cell 175, 85-100.e123.
Jimenez-Boj, E., Stamm, T. A., Sadlonova, M., Rovensky, J., Raffayova, H., Leeb, B., Machold, K. P., Graninger, W. B., and Smolen, J. S. (2012). Rituximab in psoriatic arthritis: an exploratory evaluation. Annals of the rheumatic diseases 71, 1868-1871.
Jin, H., Ni, X., Deng, R., Song, Q., Young, J., Cassady, K., Zhang, M., Forman, S., Martin, P. J., Liu, Q., et al. (2016). Antibodies from donor B cells perpetuate cutaneous chronic graft-versus-host disease in mice. Blood 127, 2249-2260.
Jin, L., Yu, D., Li, X., Yu, N., Li, X., Wang, Y., and Wang, Y. (2014). CD4+CXCR5+ follicular helper T cells in salivary gland promote B cells maturation in patients with primary Sjogren's syndrome. International journal of clinical and experimental pathology 7, 1988-1996.
Jolles, S., Borrell, R., Zouwail, S., Heaps, A., Sharp, H., Moody, M., Selwood, C., Williams, P., Phillips, C., Hood, K., et al. (2014). Calculated globulin (CG) as a screening test for antibody deficiency. Clinical and experimental immunology 177, 671-678.
Jordan, S., Distler, J. H., Maurer, B., Huscher, D., van Laar, J. M., Allanore, Y., and Distler, O. (2015). Effects and safety of rituximab in systemic sclerosis: an analysis from the European Scleroderma Trial and Research (EUSTAR) group. Annals of the rheumatic diseases 74, 1188-1194.
Joshi, R. S., Quadros, R., Drumm, M., Ain, R., and Panicker, M. M. (2017). Sedative effect of Clozapine is a function of 5-HT2A and environmental novelty. European neuropsychopharmacology: the journal of the European College of Neuropsychopharmacology 27, 70-81.
Jurickova, I., Collins, M. H., Chalk, C., Seese, A., Bezold, R., Lake, K., von Allmen, D., Frischer, J. S., Falcone, R. A., Trapnell, B. C., et al. (2013). Paediatric Crohn disease patients with stricturing behaviour exhibit ileal granulocyte-macrophage colony-stimulating factor (GM-CSF) autoantibody production and reduced neutrophil bacterial killing and GM-CSF bioactivity. Clinical and experimental immunology 172, 455-465.
Kahlert, K., Gran, F., Muhammad, K., Benoit, S., Serfling, E., Goebeler, M., and Kerstan, A. (2018). Aberrant B Cell Subsets and Immunoglobulin Levels in Patients with Moderate-to-severe Psoriasis. Acta dermato-venereologica.
Kappos, L., Hartung, H. P., Freedman, M. S., Boyko, A., Radu, E. W., Mikol, D. D., Lamarine, M., Hyvert, Y., Freudensprung, U., Plitz, T., et al. (2014). Atacicept in multiple sclerosis (ATAMS): a randomised, placebo-controlled, double-blind, phase 2 trial. The Lancet Neurology 13, 353-363.
Karlsen, T. H., Folseraas, T., Thorburn, D., and Vesterhus, M. (2017). Primary sclerosing cholangitis—a comprehensive review. Journal of hepatology 67, 1298-1323.
Karpatkin, S., and Siskind, G. W. (1969). In vitro detection of platelet antibody in patients with idiopathic thrombocytopenic purpura and systemic lupus erythematosus. Blood 33, 795-812.
Kaul, A., Gordon, C., Crow, M. K., Touma, Z., Urowitz, M. B., van Vollenhoven, R., Ruiz-Irastorza, G., and Hughes, G. (2016). Systemic lupus erythematosus. Nature reviews Disease primers 2, 16039.
Keegan, M., Konig, F., McClelland, R., Bruck, W., Morales, Y., Bitsch, A., Panitch, H., Lassmann, H., Weinshenker, B., Rodriguez, M., et al. (2005). Relation between humoral pathological changes in multiple sclerosis and response to therapeutic plasma exchange. Lancet (London, England) 366, 579-582.
Keir, G. J., Maher, T. M., Hansell, D. M., Denton, C. P., Ong, V. H., Singh, S., Wells, A. U., and Renzoni, E. A. (2012). Severe interstitial lung disease in connective tissue disease: rituximab as rescue therapy. The European respiratory journal 40, 641-648.
Keir, G. J., Maher, T. M., Ming, D., Abdullah, R., de Lauretis, A., Wickremasinghe, M., Nicholson, A. G., Hansell, D. M., Wells, A. U., and Renzoni, E. A. (2014). Rituximab in severe, treatment-refractory interstitial lung disease. Respirology (Carlton, Vic) 19, 353-359.
Kemp, E. H., Gavalas, N. G., Gawkrodger, D. J., and Weetman, A. P. (2007). Autoantibody responses to melanocytes in the depigmenting skin disease vitiligo. Autoimmunity reviews 6, 138-142.
Kemp, E. H., Waterman, E. A., Hawes, B. E., O'Neill, K., Gottumukkala, R. V., Gawkrodger, D. J., Weetman, A. P., and Watson, P. F. (2002). The melanin-concentrating hormone receptor 1, a novel target of autoantibody responses in vitiligo. The Journal of clinical investigation 109, 923-930.
Khellaf, M., Charles-Nelson, A., Fain, O., Terriou, L., Viallard, J. F., Cheze, S., Graveleau, J., Slama, B., Audia, S., Ebbo, M., et al. (2014). Safety and efficacy of rituximab in adult immune thrombocytopenia: results from a prospective registry including 248 patients. Blood 124, 3228-3236.
Khoury, E. L., Hammond, L., Bottazzo, G. F., and Doniach, D. (1981). Surface-reactive antibodies to human adrenal cells in Addison's disease. Clinical and experimental immunology 45, 48-55.
Kienbaum, M., Koy, C., Montgomery, H. V., Drynda, S., Lorenz, P., Illges, H., Tanaka, K., Kekow, J., Guthke, R., Thiesen, H. J., et al. (2009). MS characterization of apheresis samples from rheumatoid arthritis patients for the improvement of immunoadsorption therapy—a pilot study. Proteomics Clinical applications 3, 797-809.
Kil, L. P., de Bruijn, M. J., van Nimwegen, M., Corneth, O. B., van Hamburg, J. P., Dingjan, G. M., Thaiss, F., Rimmelzwaan, G. F., Elewaut, D., Delsing, D., et al. (2012). Btk levels set the threshold for B-cell activation and negative selection of autoreactive B cells in mice. Blood 119, 3744-3756.
Kim, Y. U., Lim, H., Jung, H. E., Wetsel, R. A., and Chung, Y. (2015). Regulation of autoimmune germinal center reactions in lupus-prone BXD2 mice by follicular helper T cells. PloS one 10, e0120294.
Kind, P., Lipsky, P. E., and Sontheimer, R. D. (1986). Circulating T- and B-cell abnormalities in cutaneous lupus erythematosus. The Journal of investigative dermatology 86, 235-239.
Kissel, J. T., Mendell, J. R., and Rammohan, K. W. (1986). Microvascular deposition of complement membrane attack complex in dermatomyositis. The New England journal of medicine 314, 329-334.
Kleinau, S., Martinsson, P., and Heyman, B. (2000). Induction and suppression of collagen-induced arthritis is dependent on distinct fcgamma receptors. The Journal of experimental medicine 191, 1611-1616.
Kracker, S., and Durandy, A. (2011). Insights into the B cell specific process of immunoglobulin class switch recombination. Immunology letters 138, 97-103.
Krishnamurthy, A., Joshua, V., Haj Hensvold, A., Jin, T., Sun, M., Vivar, N., Ytterberg, A. J., Engstrom, M., Fernandes-Cerqueira, C., Amara, K., et al. (2016). Identification of a novel chemokine-dependent molecular mechanism underlying rheumatoid arthritis-associated autoantibody-mediated bone loss. Annals of the rheumatic diseases 75, 721-729.
Kronbichler, A., Brezina, B., Quintana, L. F., and Jayne, D. R. (2016). Efficacy of plasma exchange and immunoadsorption in systemic lupus erythematosus and antiphospholipid syndrome: A systematic review. Autoimmunity reviews 15, 38-49.
Krumbholz, M., Derfuss, T., Hohlfeld, R., and Meinl, E. (2012). B cells and antibodies in multiple sclerosis pathogenesis and therapy. Nature reviews Neurology 8, 613-623.
Krystufkova, O., Barbasso Helmers, S., Venalis, P., Malmstrom, V., Lindroos, E., Vencovsky, J., and Lundberg, I. E. (2014). Expression of BAFF receptors in muscle tissue of myositis patients with anti-Jo-1 or anti-Ro52/anti-Ro60 autoantibodies. Arthritis research & therapy 16, 454.
Kubo, S., Nakayamada, S., Zhao, J., Yoshikawa, M., Miyazaki, Y., Nawata, A., Hirata, S., Nakano, K., Saito, K., and Tanaka, Y. (2018). Correlation of T follicular helper cells and plasmablasts with the development of organ involvement in patients with IgG4-related disease. Rheumatology (Oxford, England) 57, 514-524.
Kuchen, S., Robbins, R., Sims, G. P., Sheng, C., Phillips, T. M., Lipsky, P. E., and Ettinger, R. (2007). Essential role of IL-21 in B cell activation, expansion, and plasma cell generation during CD4⁺ T cell-B cell collaboration. Journal of immunology (Baltimore, Md.: 1950) 179, 5886-5896.
Kuenz, B., Lutterotti, A., Ehling, R., Gneiss, C., Haemmerle, M., Rainer, C., Deisenhammer, F., Schocke, M., Berger, T., and Reindl, M. (2008). Cerebrospinal fluid B cells correlate with early brain inflammation in multiple sclerosis. PloS one 3, e2559.
Kuhn, K. A., Kulik, L., Tomooka, B., Braschler, K. J., Arend, W. P., Robinson, W. H., and Holers, V. M. (2006). Antibodies against citrullinated proteins enhance tissue injury in experimental autoimmune arthritis. The Journal of clinical investigation 116, 961-973.
Kumar, V., Gutierrez-Achury, J., Kanduri, K., Almeida, R., Hrdlickova, B., Zhernakova, D. V., Westra, H. J., Karjalainen, J., Ricano-Ponce, I., Li, Y., et al. (2015). Systematic annotation of celiac disease loci refines pathological pathways and suggests a genetic explanation for increased interferon-gamma levels. Human molecular genetics 24, 397-409.
Kuo, C. J., Yang, S. Y., Liao, Y. T., Chen, W. J., Lee, W. C., Shau, W. Y., Chang, Y. T., Tsai, S. Y., and Chen, C. C. (2013). Second-generation antipsychotic medications and risk of pneumonia in schizophrenia. Schizophrenia bulletin 39, 648-657.
Kurosu, K., Takiguchi, Y., Okada, O., Yumoto, N., Sakao, S., Tada, Y., Kasahara, Y., Tanabe, N., Tatsumi, K., Weiden, M., et al. (2008). Identification of annexin 1 as a novel autoantigen in acute exacerbation of idiopathic pulmonary fibrosis. Journal of immunology (Baltimore, Md.: 1950) 181, 756-767.
Lancaster-Smith, M., Joyce, S., and Kumar, P. (1977). Immunoglobulins in the jejunal mucosa in adult coeliac disease and dermatitis herpetiformis after the reintroduction of dietary gluten. Gut 18, 887-891.
Laurent, L., Clavel, C., Lemaire, O., Anquetil, F., Cornillet, M., Zabraniecki, L., Nogueira, L., Fournie, B., Serre, G., and Sebbag, M. (2011). Fcgamma receptor profile of monocytes and macrophages from rheumatoid arthritis patients and their response to immune complexes formed with autoantibodies to citrullinated proteins. Annals of the rheumatic diseases 70, 1052-1059.
Laureti, S., De Bellis, A., Muccitelli, V. I., Calcinaro, F., Bizzarro, A., Rossi, R., Bellastella, A., Santeusanio, F., and Falorni, A. (1998). Levels of adrenocortical autoantibodies correlate with the degree of adrenal dysfunction in subjects with preclinical Addison's disease. The Journal of clinical endocrinology and metabolism 83, 3507-3511.
Lavie, F., Miceli-Richard, C., Quillard, J., Roux, S., Leclerc, P., and Mariette, X. (2004). Expression of BAFF (BLyS) in T cells infiltrating labial salivary glands from patients with Sjogren's syndrome. The Journal of pathology 202, 496-502.
Lee, S. K., Rigby, R. J., Zotos, D., Tsai, L. M., Kawamoto, S., Marshall, J. L., Ramiscal, R. R., Chan, T. D., Gatto, D., Brink, R., et al. (2011). B cell priming for extrafollicular antibody responses requires Bcl-6 expression by T cells. The Journal of experimental medicine 208, 1377-1388.
Leiper, K., Martin, K., Ellis, A., Subramanian, S., Watson, A. J., Christmas, S. E., Howarth, D., Campbell, F., and Rhodes, J. M. (2011). Randomised placebo-controlled trial of rituximab (anti-CD20) in active ulcerative colitis. Gut 60, 1520-1526.
Leonardo, S. M., De Santis, J. L., Gehrand, A., Malherbe, L. P., and Gauld, S. B. (2012). Expansion of follicular helper T cells in the absence of Treg cells: implications for loss of B-cell anergy. European journal of immunology 42, 2597-2607.
Lepri, G., Avouac, J., Airo, P., Anguita Santos, F., Bellando-Randone, S., Blagojevic, J., Garcia Hernandez, F., Gonzalez Nieto, J. A., Guiducci, S., Jordan, S., et al. (2016). Effects of rituximab in connective tissue disorders related interstitial lung disease. Clinical and experimental rheumatology 34 Suppl 100, 181-185.
Leung, J. G., Hasassri, M. E., Barreto, J. N., Nelson, S., and Morgan, R. J., 3rd (2017). Characterization of Admission Types in Medically Hospitalized Patients Prescribed Clozapine. Psychosomatics 58, 164-172.
Li, H., Fang, M., Xu, M., Li, S., Du, J., Li, W., and Chen, H. (2016a). Chronic Olanzapine Treatment Induces Disorders of Plasma Fatty Acid Profile in Balb/c Mice: A Potential Mechanism for Olanzapine-Induced Insulin Resistance. PloS one 11, e0167930.
Li, Y., Yang, M., Zhang, R., Liu, W., Zhang, K., Wen, W., Yi, L., Wang, Q., Hao, M., Yang, H., et al. (2016b). Evaluation of serum immunoglobulins concentrations and distributions in vitiligo patients. Immunologic research 64, 1150-1156.
Ligocki, A. J., Rounds, W. H., Cameron, E. M., Harp, C. T., Frohman, E. M., Courtney, A. M., Vernino, S., Cowell, L. G., Greenberg, B., and Monson, N. L. (2013). Expansion of CD27high plasmablasts in transverse myelitis patients that utilize VH4 and JH6 genes and undergo extensive somatic hypermutation. Genes and immunity 14, 291-301.
Lili, Y., Yi, W., Ji, Y., Yue, S., Weimin, S., and Ming, L. (2012). Global activation of CD8+ cytotoxic T lymphocytes correlates with an impairment in regulatory T cells in patients with generalized vitiligo. PloS one 7, e37513.
Lin, W., Zhang, P., Chen, H., Chen, Y., Yang, H., Zheng, W., Zhang, X., Zhang, F., Zhang, W., and Lipsky, P. E. (2017). Circulating plasmablasts/plasma cells: a potential biomarker for IgG4-related disease. Arthritis research & therapy 19, 25.
Lindfors, K., Ciacci, C., Kurppa, K., Lundin, K. E. A., Makharia, G. K., Mearin, M. L., Murray, J. A., Verdu, E. F., and Kaukinen, K. (2019). Coeliac disease. Nature reviews Disease primers 5, 3.
Linterman, M. A., Pierson, W., Lee, S. K., Kallies, A., Kawamoto, S., Rayner, T. F., Srivastava, M., Divekar, D. P., Beaton, L., Hogan, J. J., et al. (2011). Foxp3⁺ follicular regulatory T cells control the germinal center response. Nature medicine 17, 975-982.
Liu, C. W., and Huang, Y. C. (2018). Vitiligo and autoantibodies: a systematic review and meta-analysis. Journal der Deutschen Dermatologischen Gesellschaft=Journal of the German Society of Dermatology: JDDG 16, 845-851.
Liu, D., Xu, H., Shih, C., Wan, Z., Ma, X., Ma, W., Luo, D., and Qi, H. (2015). T-B-cell entanglement and ICOSL-driven feed-forward regulation of germinal centre reaction. Nature 517, 214-218.
Longbrake, E. E., and Cross, A. H. (2016). Effect of Multiple Sclerosis Disease-Modifying Therapies on B Cells and Humoral Immunity. JAMA neurology 73, 219-225.
Lopez De Padilla, C. M., McNallan, K. T., Crowson, C. S., Bilgic, H., Bram, R. J., Hein, M. S., Ytterberg, S. R., Amin, S., Peterson, E. J., Baechler, E. C., et al. (2013). BAFF expression correlates with idiopathic inflammatory myopathy disease activity measures and autoantibodies. The Journal of rheumatology 40, 294-302.
Lota, H. K., Keir, G. J., Hansell, D. M., Nicholson, A. G., Maher, T. M., Wells, A. U., and Renzoni, E. A. (2013). Novel use of rituximab in hypersensitivity pneumonitis refractory to conventional treatment. Thorax 68, 780-781.
Lovato, L., Willis, S. N., Rodig, S. J., Caron, T., Almendinger, S. E., Howell, O. W., Reynolds, R., O'Connor, K. C., and Hafler, D. A. (2011). Related B cell clones populate the meninges and parenchyma of patients with multiple sclerosis. Brain: a journal of neurology 134, 534-541.
Lozano, R., Marin, R., Santacruz, M. J., and Pascual, A. (2016). Effect of clozapine on immunoglobulin M plasma levels. Therapeutic advances in psychopharmacology 6, 58-60.
Magro, C. M., Waldman, W. J., Knight, D. A., Allen, J. N., Nadasdy, T., Frambach, G. E., Ross, P., and Marsh, C. B. (2006). Idiopathic pulmonary fibrosis related to endothelial injury and antiendothelial cell antibodies. Human immunology 67, 284-297.
Mahevas, M., Patin, P., Huetz, F., Descatoire, M., Cagnard, N., Bole-Feysot, C., Le Gallou, S., Khellaf, M., Fain, O., Boutboul, D., et al. (2013). B cell depletion in immune thrombocytopenia reveals splenic long-lived plasma cells. The Journal of clinical investigation 123, 432-442.
Malard, F., Labopin, M., Yakoub-Agha, I., Chantepie, S., Guillaume, T., Blaise, D., Tabrizi, R., Magro, L., Vanhove, B., Blancho, G., et al. (2017). Rituximab-based first-line treatment of cGVHD after allogeneic SCT: results of a phase 2 study. Blood 130, 2186-2195.
Maleki, A., Lamba, N., Ma, L., Lee, S., Schmidt, A., and Foster, C. S. (2017). Rituximab as a monotherapy or in combination therapy for the treatment of non-paraneoplastic autoimmune retinopathy. Clinical ophthalmology (Auckland, NZ) 11, 377-385.
Mankarious, S., Lee, M., Fischer, S., Pyun, K. H., Ochs, H. D., Oxelius, V. A., and Wedgwood, R. J. (1988). The half-lives of IgG subclasses and specific antibodies in patients with primary immunodeficiency who are receiving intravenously administered immunoglobulin. The Journal of laboratory and clinical medicine 112, 634-640.
Marchal-Somme, J., Uzunhan, Y., Marchand-Adam, S., Valeyre, D., Soumelis, V., Crestani, B., and Soler, P. (2006). Cutting edge: nonproliferating mature immune cells form a novel type of organized lymphoid structure in idiopathic pulmonary fibrosis. Journal of immunology (Baltimore, Md.: 1950) 176, 5735-5739.
Mariette, X., Seror, R., Quartuccio, L., Baron, G., Salvin, S., Fabris, M., Desmoulins, F., Nocturne, G., Ravaud, P., and De Vita, S. (2015). Efficacy and safety of belimumab in primary Sjogren's syndrome: results of the BELISS open-label phase II study. Annals of the rheumatic diseases 74, 526-531.
Marinkovic, D., Timotijevic, I., Babinski, T., Totic, S., and Paunovic, V. R. (1994). The side-effects of clozapine: a four year follow-up study. Progress in neuro-psychopharmacology & biological psychiatry 18, 537-544.
Marino, E., Tan, B., Binge, L., Mackay, C. R., and Grey, S. T. (2012). B-cell cross-presentation of autologous antigen precipitates diabetes. Diabetes 61, 2893-2905.
Mathey, E. K., Derfuss, T., Storch, M. K., Williams, K. R., Hales, K., Woolley, D. R., Al-Hayani, A., Davies, S. N., Rasband, M. N., Olsson, T., et al. (2007). Neurofascin as a novel target for autoantibody-mediated axonal injury. The Journal of experimental medicine 204, 2363-2372.
Matsumoto, M., Baba, A., Yokota, T., Nishikawa, H., Ohkawa, Y., Kayama, H., Kallies, A., Nutt, S. L., Sakaguchi, S., Takeda, K., et al. (2014). Interleukin-10-producing plasmablasts exert regulatory function in autoimmune inflammation. Immunity 41, 1040-1051.
Matsushita, T., Hasegawa, M., Yanaba, K., Kodera, M., Takehara, K., and Sato, S. (2006). Elevated serum BAFF levels in patients with systemic sclerosis: enhanced BAFF signaling in systemic sclerosis B lymphocytes. Arthritis and rheumatism 54, 192-201.
Matysiak-Budnik, T., Moura, I. C., Arcos-Fajardo, M., Lebreton, C., Menard, S., Candalh, C., Ben-Khalifa, K., Dugave, C., Tamouza, H., van Niel, G., et al. (2008). Secretory IgA mediates retrotranscytosis of intact gliadin peptides via the transferrin receptor in celiac disease. The Journal of experimental medicine 205, 143-154.
Mavropoulos, A., Varna, A., Zafiriou, E., Liaskos, C., Alexiou, I., Roussaki-Schulze, A., Vlychou, M., Katsiari, C., Bogdanos, D. P., and Sakkas, L. I. (2017). IL-10 producing Bregs are impaired in psoriatic arthritis and psoriasis and inversely correlate with IL-17- and IFNgamma-producing T cells. Clinical immunology (Orlando, Fla.) 184, 33-41.
Mayer, M. C., and Meinl, E. (2012). Glycoproteins as targets of autoantibodies in CNS inflammation: MOG and more. Therapeutic advances in neurological disorders 5, 147-159.
McClain, M. T., Arbuckle, M. R., Heinlen, L. D., Dennis, G. J., Roebuck, J., Rubertone, M. V., Harley, J. B., and James, J. A. (2004). The prevalence, onset, and clinical significance of antiphospholipid antibodies prior to diagnosis of systemic lupus erythematosus. Arthritis and rheumatism 50, 1226-1232.
McIntyre, D., Zuckerman, N. S., Field, M., Mehr, R., and Stott, D. I. (2014). The V(H) repertoire and clonal diversification of B cells in inflammatory myopathies. European journal of immunology 44, 585-596.
McMillan, R., Luiken, G. A., Levy, R., Yelenosky, R., and Longmire, R. L. (1978). Antibody against megakaryocytes in idiopathic thrombocytopenic purpura. Jama 239, 2460-2462.
McMillan, R., Wang, L., and Tani, P. (2003). Prospective evaluation of the immunobead assay for the diagnosis of adult chronic immune thrombocytopenic purpura (ITP). Journal of thrombosis and haemostasis: JTH 1, 485-491.
McMillan, R., Wang, L., Tomer, A., Nichol, J., and Pistillo, J. (2004). Suppression of in vitro megakaryocyte production by antiplatelet autoantibodies from adult patients with chronic ITP. Blood 103, 1364-1369.
McOmish, C. E., Lira, A., Hanks, J. B., and Gingrich, J. A. (2012). Clozapine-induced locomotor suppression is mediated by 5-HT2A receptors in the forebrain. Neuropsychopharmacology: official publication of the American College of Neuropsychopharmacology 37, 2747-2755.
Means, T. K., and Luster, A. D. (2005). Toll-like receptor activation in the pathogenesis of systemic lupus erythematosus. Annals of the New York Academy of Sciences 1062, 242-251.
Mei, H. E., Yoshida, T., Sime, W., Hiepe, F., Thiele, K., Manz, R. A., Radbruch, A., and Dorner, T. (2009). Blood-borne human plasma cells in steady state are derived from mucosal immune responses. Blood 113, 2461-2469.
Melchers, F. (2015). Checkpoints that control B cell development. The Journal of clinical investigation 125, 2203-2210.
Menard, L., Samuels, J., Ng, Y. S., and Meffre, E. (2011). Inflammation-independent defective early B cell tolerance checkpoints in rheumatoid arthritis. Arthritis and rheumatism 63, 1237-1245.
Meyer-Hermann, M., Mohr, E., Pelletier, N., Zhang, Y., Victora, G. D., and Toellner, K. M. (2012). A theory of germinal center B cell selection, division, and exit. Cell reports 2, 162-174.
Miklos, D. B., Kim, H. T., Miller, K. H., Guo, L., Zorn, E., Lee, S. J., Hochberg, E. P., Wu, C. J., Alyea, E. P., Cutler, C., et al. (2005). Antibody responses to H-Y minor histocompatibility antigens correlate with chronic graft-versus-host disease and disease remission. Blood 105, 2973-2978.
Millan, M. J., Audinot, V., Melon, C., and Newman-Tancredi, A. (1995). Evidence that dopamine D3 receptors participate in clozapine-induced hypothermia. European journal of pharmacology 280, 225-229.
Mitchell, A. L., and Pearce, S. H. (2012). Autoimmune Addison disease: pathophysiology and genetic complexity. Nature reviews Endocrinology 8, 306-316.
Mittereder, N., Kuta, E., Bhat, G., Dacosta, K., Cheng, L. I., Herbst, R., and Carlesso, G. (2016). Loss of Immune Tolerance Is Controlled by ICOS in Sle1 Mice. Journal of immunology (Baltimore, Md.: 1950) 197, 491-503.
Mok, C. C., Ho, L. Y., and To, C. H. (2007). Rituximab for refractory polymyositis: an open-label prospective study. The Journal of rheumatology 34, 1864-1868.
Montalban, X., Hauser, S. L., Kappos, L., Arnold, D. L., Bar-Or, A., Comi, G., de Seze, J., Giovannoni, G., Hartung, H. P., Hemmer, B., et al. (2017). Ocrelizumab versus Placebo in Primary Progressive Multiple Sclerosis. The New England journal of medicine 376, 209-220.
Moreno, J. L., Holloway, T., Umali, A., Rayannavar, V., Sealfon, S. C., and Gonzalez-Maeso, J. (2013). Persistent effects of chronic clozapine on the cellular and behavioral responses to LSD in mice. Psychopharmacology 225, 217-226.
Morita, R., Schmitt, N., Bentebibel, S. E., Ranganathan, R., Bourdery, L., Zurawski, G., Foucat, E., Dullaers, M., Oh, S., Sabzghabaei, N., et al. (2011). Human blood CXCR5(+)CD4(+) T cells are counterparts of T follicular cells and contain specific subsets that differentially support antibody secretion. Immunity 34, 108-121.
Mosca, M., Tani, C., Vagnani, S., Carli, L., and Bombardieri, S. (2014). The diagnosis and classification of undifferentiated connective tissue diseases. Journal of autoimmunity 48-49, 50-52.
Moschovakis, G. L., Bubke, A., Friedrichsen, M., Falk, C. S., Feederle, R., and Forster, R. (2017). T cell specific Cxcr5 deficiency prevents rheumatoid arthritis. Scientific reports 7, 8933.
Moura, R. A., Cascao, R., Perpetuo, I., Canhao, H., Vieira-Sousa, E., Mourao, A. F., Rodrigues, A. M., Polido-Pereira, J., Queiroz, M. V., Rosario, H. S., et al. (2011). Cytokine pattern in very early rheumatoid arthritis favours B-cell activation and survival. Rheumatology (Oxford, England) 50, 278-282.
Mumtaz, I. M., Hoyer, B. F., Panne, D., Moser, K., Winter, O., Cheng, Q. Y., Yoshida, T., Burmester, G. R., Radbruch, A., Manz, R. A., et al. (2012). Bone marrow of NZB/W mice is the major site for plasma cells resistant to dexamethasone and cyclophosphamide: implications for the treatment of autoimmunity. Journal of autoimmunity 39, 180-188.
Mutlu, O., Gumuslu, E., Ulak, G., Celikyurt, I. K., Kokturk, S., Kir, H. M., Akar, F., and Erden, F. (2012). Effects of fluoxetine, tianeptine and olanzapine on unpredictable chronic mild stress-induced depression-like behavior in mice. Life sciences 91, 1252-1262.
Muto, A., Ochiai, K., Kimura, Y., Itoh-Nakadai, A., Calame, K. L., Ikebe, D., Tashiro, S., and Igarashi, K. (2010). Bach2 represses plasma cell gene regulatory network in B cells to promote antibody class switch. The EMBO journal 29, 4048-4061.
Muto, A., Tashiro, S., Nakajima, O., Hoshino, H., Takahashi, S., Sakoda, E., Ikebe, D., Yamamoto, M., and Igarashi, K. (2004). The transcriptional programme of antibody class switching involves the repressor Bach2. Nature 429, 566-571.
Myers, R. P., Swain, M. G., Lee, S. S., Shaheen, A. A., and Burak, K. W. (2013). B-cell depletion with rituximab in patients with primary biliary cirrhosis refractory to ursodeoxycholic acid. The American journal of gastroenterology 108, 933-941.
Myrsky, E., Kaukinen, K., Syrjanen, M., Korponay-Szabo, I. R., Maki, M., and Lindfors, K. (2008). Coeliac disease-specific autoantibodies targeted against transglutaminase 2 disturb angiogenesis. Clinical and experimental immunology 152, 111-119.
Naito, Y., Takematsu, H., Koyama, S., Miyake, S., Yamamoto, H., Fujinawa, R., Sugai, M., Okuno, Y., Tsujimoto, G., Yamaji, T., et al. (2007). Germinal center marker GL7 probes activation-dependent repression of N-glycolylneuraminic acid, a sialic acid species involved in the negative modulation of B-cell activation. Molecular and cellular biology 27, 3008-3022.
Nakamura, M. (2014). Clinical significance of autoantibodies in primary biliary cirrhosis. Seminars in liver disease 34, 334-340.
Nakayamada, S., Kubo, S., Yoshikawa, M., Miyazaki, Y., Yunoue, N., Iwata, S., Miyagawa, I., Hirata, S., Nakano, K., Saito, K., et al. (2018). Differential effects of biological DMARDs on peripheral immune cell phenotypes in patients with rheumatoid arthritis. Rheumatology (Oxford, England) 57, 164-174.
Nandakumar, K. S., Backlund, J., Vestberg, M., and Holmdahl, R. (2004). Collagen type II (CII)-specific antibodies induce arthritis in the absence of T or B cells but the arthritis progression is enhanced by CII-reactive T cells. Arthritis research & therapy 6, R544-550.
Nguyen, D. C., Garimalla, S., Xiao, H., Kyu, S., Albizua, I., Galipeau, J., Chiang, K. Y., Waller, E. K., Wu, R., Gibson, G., et al. (2018). Factors of the bone marrow microniche that support human plasma cell survival and immunoglobulin secretion. Nature communications 9, 3698.
Nguyen, Q. D., Humphrey, R. L., Dunn, J. P., and Humayun, M. S. (2001). Elevated vitreous concentration of monoclonal immunoglobulin manifesting as schlieren in juvenile rheumatoid arthritis-associated uveitis. Archives of ophthalmology (Chicago, Ill.: 1960) 119, 293-296.
Nihtyanova, S. I., and Denton, C. P. (2010). Autoantibodies as predictive tools in systemic sclerosis. Nature reviews Rheumatology 6, 112-116.
Nikiphorou, E., and Hall, F. C. (2014). First report of improvement of coeliac disease in a patient with Sjogren's syndrome treated with rituximab. Rheumatology (Oxford, England) 53, 1906-1907.
Niu, J., Song, Z., Yang, X., Zhai, Z., Zhong, H., and Hao, F. (2015). Increased circulating follicular helper T cells and activated B cells correlate with disease severity in patients with psoriasis. Journal of the European Academy of Dermatology and Venereology: JEADV 29, 1791-1796.
Noorchashm, H., Noorchashm, N., Kern, J., Rostami, S. Y., Barker, C. F., and Naji, A. (1997). B-cells are required for the initiation of insulitis and sialitis in nonobese diabetic mice. Diabetes 46, 941-946.
Norris, D. A., Kissinger, R. M., Naughton, G. M., and Bystryn, J. C. (1988). Evidence for immunologic mechanisms in human vitiligo: patients' sera induce damage to human melanocytes in vitro by complement-mediated damage and antibody-dependent cellular cytotoxicity. The Journal of investigative dermatology 90, 783-789.
Nugent, D., McMillan, R., Nichol, J. L., and Slichter, S. J. (2009). Pathogenesis of chronic immune thrombocytopenia: increased platelet destruction and/or decreased platelet production. British journal of haematology 146, 585-596.
Nutt, S. L., Hodgkin, P. D., Tarlinton, D. M., and Corcoran, L. M. (2015). The generation of antibody-secreting plasma cells. Nature reviews Immunology 15, 160-171.
O'Dwyer, R., Kovaleva, M., Zhang, J., Steven, J., Cummins, E., Luxenberg, D., Darmanin-Sheehan, A., Carvalho, M. F., Whitters, M., Saunders, K., et al. (2018). Anti-ICOSL New Antigen Receptor Domains Inhibit T Cell Proliferation and Reduce the Development of Inflammation in the Collagen-Induced Mouse Model of Rheumatoid Arthritis. Journal of immunology research 2018, 4089459.
Obermeier, B., Mentele, R., Malotka, J., Kellermann, J., Kumpfel, T., Wekerle, H., Lottspeich, F., Hohlfeld, R., and Dornmair, K. (2008). Matching of oligoclonal immunoglobulin transcriptomes and proteomes of cerebrospinal fluid in multiple sclerosis. Nature medicine 14, 688-693.
Oddis, C. V., Reed, A. M., Aggarwal, R., Rider, L. G., Ascherman, D. P., Levesque, M. C., Barohn, R. J., Feldman, B. M., Harris-Love, M. O., Koontz, D. C., et al. (2013). Rituximab in the treatment of refractory adult and juvenile dermatomyositis and adult polymyositis: a randomized, placebo-phase trial. Arthritis and rheumatism 65, 314-324.
Okawa-Takatsuji, M., Aotsuka, S., Uwatoko, S., Takaono, M., Iwasaki, K., Kinoshita, M., and Sumiya, M. (2001). Endothelial cell-binding activity of anti-U1-ribonucleoprotein antibodies in patients with connective tissue diseases. Clinical and experimental immunology 126, 345-354.
Olalekan, S. A., Cao, Y., Hamel, K. M., and Finnegan, A. (2015). B cells expressing IFN-gamma suppress Treg-cell differentiation and promote autoimmune experimental arthritis. European journal of immunology 45, 988-998.
Orange, J. S., Ballow, M., Stiehm, E. R., Ballas, Z. K., Chinen, J., De La Morena, M., Kumararatne, D., Harville, T. O., Hesterberg, P., Koleilat, M., et al. (2012). Use and interpretation of diagnostic vaccination in primary immunodeficiency: a working group report of the Basic and Clinical Immunology Interest Section of the American Academy of Allergy, Asthma & Immunology. The Journal of allergy and clinical immunology 130, S1-24.
Owczarczyk, K., Lal, P., Abbas, A. R., Wolslegel, K., Holweg, C. T., Dummer, W., Kelman, A., Brunetta, P., Lewin-Koh, N., Sorani, M., et al. (2011). A plasmablast biomarker for nonresponse to antibody therapy to CD20 in rheumatoid arthritis. Science translational medicine 3, 101ra192.
Pabst, O. (2012). New concepts in the generation and functions of IgA. Nature reviews Immunology 12, 821-832.
Palanichamy, A., Apeltsin, L., Kuo, T. C., Sirota, M., Wang, S., Pitts, S. J., Sundar, P. D., Telman, D., Zhao, L. Z., Derstine, M., et al. (2014). Immunoglobulin class-switched B cells form an active immune axis between CNS and periphery in multiple sclerosis. Science translational medicine 6, 248ra106.
Panneton, V., Bagherzadeh Yazdchi, S., Witalis, M., Chang, J., and Suh, W. K. (2018). ICOS Signaling Controls Induction and Maintenance of Collagen-Induced Arthritis. Journal of immunology (Baltimore, Md.: 1950) 200, 3067-3076.
Parackova, Z., Klocperk, A., Rataj, M., Kayserova, J., Zentsova, I., Sumnik, Z., Kolouskova, S., Sklenarova, J., Pruhova, S., Obermannova, B., et al. (2017). Alteration of B cell subsets and the receptor for B cell activating factor (BAFF) in paediatric patients with type 1 diabetes. Immunology letters 189, 94-100.
Pazderska, A., Oftedal, B. E., Napier, C. M., Ainsworth, H. F., Husebye, E. S., Cordell, H. J., Pearce, S. H., and Mitchell, A. L. (2016). A Variant in the BACH2 Gene Is Associated With Susceptibility to Autoimmune Addison's Disease in Humans. The Journal of clinical endocrinology and metabolism 101, 3865-3869.
Pearce, S. H., Mitchell, A. L., Bennett, S., King, P., Chandran, S., Nag, S., Chen, S., Smith, B. R., Isaacs,
J. D., and Vaidya, B. (2012). Adrenal steroidogenesis after B lymphocyte depletion therapy in new-onset Addison's disease. The Journal of clinical endocrinology and metabolism 97, E1927-1932.
Pelegrin, L., Jakob, E., Schmidt-Bacher, A., Schwenger, V., Becker, M., Max, R., Lorenz, H. M., and Mackensen, F. (2014). Experiences with rituximab for the treatment of autoimmune diseases with ocular involvement. The Journal of rheumatology 41, 84-90.
Penatti, A., Facciotti, F., De Matteis, R., Larghi, P., Paroni, M., Murgo, A., De Lucia, O., Pagani, M., Pierannunzii, L., Truzzi, M., et al. (2017). Differences in serum and synovial CD4⁺ T cells and cytokine profiles to stratify patients with inflammatory osteoarthritis and rheumatoid arthritis. Arthritis research & therapy 19, 103.
Pene, J., Gauchat, J. F., Lecart, S., Drouet, E., Guglielmi, P., Boulay, V., Delwail, A., Foster, D., Lecron, J. C., and Yssel, H. (2004). Cutting edge: IL-21 is a switch factor for the production of IgG1 and IgG3 by human B cells. Journal of immunology (Baltimore, Md.: 1950) 172, 5154-5157.
Pescovitz, M. D., Greenbaum, C. J., Krause-Steinrauf, H., Becker, D. J., Gitelman, S. E., Goland, R., Gottlieb, P. A., Marks, J. B., McGee, P. F., Moran, A. M., et al. (2009). Rituximab, B-lymphocyte depletion, and preservation of beta-cell function. The New England journal of medicine 361, 2143-2152.
Petro, J. B., Gerstein, R. M., Lowe, J., Carter, R. S., Shinners, N., and Khan, W. N. (2002). Transitional type 1 and 2 B lymphocyte subsets are differentially responsive to antigen receptor signaling. The Journal of biological chemistry 277, 48009-48019.
Pipi, E., Nayar, S., Gardner, D. H., Colafrancesco, S., Smith, C., and Barone, F. (2018). Tertiary Lymphoid Structures: Autoimmunity Goes Local. Frontiers in immunology 9, 1952.
Ponsford, M., Castle, D., Tahir, T., Robinson, R., Wade, W., Steven, R., Bramhall, K., Moody, M., Carne, E., Ford, C., et al. (2018a). Clozapine is associated with secondary antibody deficiency. The British journal of psychiatry: the journal of mental science, 1-7.
Ponsford, M., Castle, D., Tahir, T., Robinson, R., Wade, W., Steven, R., Bramhall, K., Moody, M., Carne, E., Ford, C., et al. (2018b). Clozapine is associated with secondary antibody deficiency. The British Journal of Psychiatry, 1-7.
Pontarini, E., Lucchesi, D., and Bombardieri, M. (2018). Current views on the pathogenesis of Sjogren's syndrome. Current opinion in rheumatology 30, 215-221.
Prete, M., Dammacco, R., Fatone, M. C., and Racanelli, V. (2016). Autoimmune uveitis: clinical, pathogenetic, and therapeutic features. Clinical and experimental medicine 16, 125-136.
Quelhas da Costa, R., Aguirre-Alastuey, M. E., Isenberg, D. A., and Saracino, A. M. (2018). Assessment of Response to B-Cell Depletion Using Rituximab in Cutaneous Lupus Erythematosus. JAMA dermatology 154, 1432-1440.
Quinton, J. F., Sendid, B., Reumaux, D., Duthilleul, P., Cortot, A., Grandbastien, B., Charrier, G., Targan, S. R., Colombel, J. F., and Poulain, D. (1998). Anti-Saccharomyces cerevisiae mannan antibodies combined with antineutrophil cytoplasmic autoantibodies in inflammatory bowel disease: prevalence and diagnostic role. Gut 42, 788-791.
Radke, J., Koll, R., Preusse, C., Pehl, D., Todorova, K., Schonemann, C., Allenbach, Y., Aronica, E., de Visser, M., Heppner, F. L., et al. (2018). Architectural B-cell organization in skeletal muscle identifies subtypes of dermatomyositis. Neurology® neuroimmunology & neuroinflammation 5, e451.
Read, R. W., Szalai, A. J., Vogt, S. D., McGwin, G., and Barnum, S. R. (2006). Genetic deficiency of C3 as well as CNS-targeted expression of the complement inhibitor sCrry ameliorates experimental autoimmune uveoretinitis. Experimental eye research 82, 389-394.
Rech, J., Kallert, S., Hueber, A. J., Requadt, C., Kalden, J. R., and Schulze-Koops, H. (2006). Combination of immunoadsorption and CD20 antibody therapy in a patient with mixed connective tissue disease. Rheumatology (Oxford, England) 45, 490-491.
Reinhardt, R. L., Liang, H. E., and Locksley, R. M. (2009). Cytokine-secreting follicular T cells shape the antibody repertoire. Nature immunology 10, 385-393.
Ren, G., and Adamus, G. (2004). Cellular targets of anti-alpha-enolase autoantibodies of patients with autoimmune retinopathy. Journal of autoimmunity 23, 161-167.
Richez, C., Truchetet, M. E., Schaeverbeke, T., and Bannwarth, B. (2014). Atacicept as an investigated therapy for rheumatoid arthritis. Expert opinion on investigational drugs 23, 1285-1294.
Riemekasten, G., Philippe, A., Nather, M., Slowinski, T., Muller, D. N., Heidecke, H., Matucci-Cerinic, M., Czirjak, L., Lukitsch, I., Becker, M., et al. (2011). Involvement of functional autoantibodies against vascular receptors in systemic sclerosis. Annals of the rheumatic diseases 70, 530-536.
Rivas, J. R., Ireland, S. J., Chkheidze, R., Rounds, W. H., Lim, J., Johnson, J., Ramirez, D. M., Ligocki, A. J., Chen, D., Guzman, A. A., et al. (2017). Peripheral VH4+ plasmablasts demonstrate autoreactive B cell expansion toward brain antigens in early multiple sclerosis patients. Acta neuropathologica 133, 43-60.
Rojas, O. L., Probstel, A. K., Porfilio, E. A., Wang, A. A., Charabati, M., Sun, T., Lee, D. S. W., Galicia, G., Ramaglia, V., Ward, L. A., et al. (2019). Recirculating Intestinal IgA-Producing Cells Regulate Neuroinflammation via IL-10. Cell 176, 610-624.e618.
Rolf, J., Bell, S. E., Kovesdi, D., Janas, M. L., Soond, D. R., Webb, L. M., Santinelli, S., Saunders, T., Hebeis, B., Killeen, N., et al. (2010). Phosphoinositide 3-kinase activity in T cells regulates the magnitude of the germinal center reaction. Journal of immunology (Baltimore, Md.: 1950) 185, 4042-4052.
Romme Christensen, J., Bornsen, L., Ratzer, R., Piehl, F., Khademi, M., Olsson, T., Sorensen, P. S., and Sellebjerg, F. (2013). Systemic inflammation in progressive multiple sclerosis involves follicular T-helper, Th17- and activated B-cells and correlates with progression. PloS one 8, e57820.
Rosenthal, N. S., and Farhi, D. C. (1989). Bone marrow findings in connective tissue disease. American journal of clinical pathology 92, 650-654.
Rostom, A., Dube, C., Cranney, A., Saloojee, N., Sy, R., Garritty, C., Sampson, M., Zhang, L., Yazdi, F., Mamaladze, V., et al. (2005). The diagnostic accuracy of serologic tests for celiac disease: a systematic review. Gastroenterology 128, S38-46.
Ruiz-Arguelles, A., Brito, G. J., Reyes-Izquierdo, P., Perez-Romano, B., and Sanchez-Sosa, S. (2007). Apoptosis of melanocytes in vitiligo results from antibody penetration. Journal of autoimmunity 29, 281-286.
Ruiz-Arguelles, A., Garcia-Carrasco, M., Jimenez-Brito, G., Sanchez-Sosa, S., Perez-Romano, B., Garces-Eisele, J., Camacho-Alarcon, C., Reyes-Nunez, V., Sandoval-Cruz, M., Mendoza-Pinto, C., et al. (2013). Treatment of vitiligo with a chimeric monoclonal antibody to CD20: a pilot study. Clinical and experimental immunology 174, 229-236.
Rummler, S., Althaus, K., Maak, B., and Barz, D. (2008). A case report of successful treatment with immunoadsorption onto protein A in mixed connective tissue disease in childhood. Therapeutic apheresis and dialysis: official peer-reviewed journal of the International Society for Apheresis, the Japanese Society for Apheresis, the Japanese Society for Dialysis Therapy 12, 337-342.
Sacchi, S., Novellis, V., Paolone, G., Nuzzo, T., Iannotta, M., Belardo, C., Squillace, M., Bolognesi, P., Rosini, E., Motta, Z., et al. (2017). Olanzapine, but not clozapine, increases glutamate release in the prefrontal cortex of freely moving mice by inhibiting D-aspartate oxidase activity. Scientific reports 7, 46288.
Sakaguchi, S., Sakaguchi, N., Asano, M., Itoh, M., and Toda, M. (1995). Immunologic self-tolerance maintained by activated T cells expressing IL-2 receptor alpha-chains (CD25). Breakdown of a single mechanism of self-tolerance causes various autoimmune diseases. Journal of immunology (Baltimore, Md.: 1950) 155, 1151-1164.
Sakuraba, K., Oyamada, A., Fujimura, K., Spolski, R., Iwamoto, Y., Leonard, W. J., Yoshikai, Y., and Yamada, H. (2016). Interleukin-21 signaling in B cells, but not in T cells, is indispensable for the development of collagen-induced arthritis in mice. Arthritis research & therapy 18, 188.
Salvi, M., Vannucchi, G., and Beck-Peccoz, P. (2013). Potential utility of rituximab for Graves' orbitopathy. The Journal of clinical endocrinology and metabolism 98, 4291-4299.
Samuels, J., Ng, Y. S., Coupillaud, C., Paget, D., and Meffre, E. (2005a). Human B cell tolerance and its failure in rheumatoid arthritis. Annals of the New York Academy of Sciences 1062, 116-126.
Samuels, J., Ng, Y. S., Coupillaud, C., Paget, D., and Meffre, E. (2005b). Impaired early B cell tolerance in patients with rheumatoid arthritis. The Journal of experimental medicine 201, 1659-1667.
Sarantopoulos, S., Stevenson, K. E., Kim, H. T., Cutler, C. S., Bhuiya, N. S., Schowalter, M., Ho, V. T., Alyea, E. P., Koreth, J., Blazar, B. R., et al. (2009). Altered B-cell homeostasis and excess BAFF in human chronic graft-versus-host disease. Blood 113, 3865-3874.
Sari, A., Guven, D., Armagan, B., Erden, A., Kalyoncu, U., Karadag, O., Apras Bilgen, S., Ertenli, I., Kiraz, S., and Akdogan, A. (2017). Rituximab Experience in Patients With Long-standing Systemic Sclerosis-Associated Interstitial Lung Disease: A Series of 14 Patients. Journal of clinical rheumatology: practical reports on rheumatic & musculoskeletal diseases 23, 411-415.
Sato, S., Fujimoto, M., Hasegawa, M., and Takehara, K. (2004). Altered blood B lymphocyte homeostasis in systemic sclerosis: expanded naive B cells and diminished but activated memory B cells. Arthritis and rheumatism 50, 1918-1927.
Sayin, I., Radtke, A. J., Vella, L. A., Jin, W., Wherry, E. J., Buggert, M., Betts, M. R., Herati, R. S., Germain, R. N., and Canaday, D. H. (2018). Spatial distribution and function of T follicular regulatory cells in human lymph nodes. The Journal of experimental medicine 215, 1531-1542.
Sebode, M., Peiseler, M., Franke, B., Schwinge, D., Schoknecht, T., Wortmann, F., Quaas, A., Petersen, B. S., Ellinghaus, E., Baron, U., et al. (2014). Reduced FOXP3(+) regulatory T cells in patients with primary sclerosing cholangitis are associated with IL2RA gene polymorphisms. Journal of hepatology 60, 1010-1016.
Seguchi, M., Soejima, Y., Tateishi, A., Iida, H., Yamamoto, M., Nakashima, K., Murakami, F., Ohashi, S., Yamashita, S., Maekawa, T., et al. (2000). Mixed Connective Tissue Disease with Multiple Organ Damage: Successful Treatment with Plasmapheresis. Internal Medicine 39, 1119-1122.
Selmi, C. (2014). Diagnosis and classification of autoimmune uveitis. Autoimmunity reviews 13, 591-594.
Sem, M., Molberg, O., Lund, M. B., and Gran, J. T. (2009). Rituximab treatment of the anti-synthetase syndrome: a retrospective case series. Rheumatology (Oxford, England) 48, 968-971.
Serafini, B., Rosicarelli, B., Magliozzi, R., Stigliano, E., and Aloisi, F. (2004). Detection of ectopic B-cell follicles with germinal centers in the meninges of patients with secondary progressive multiple sclerosis. Brain pathology (Zurich, Switzerland) 14, 164-174.
Serreze, D. V., Fleming, S. A., Chapman, H. D., Richard, S. D., Leiter, E. H., and Tisch, R. M. (1998). B lymphocytes are critical antigen-presenting cells for the initiation of T cell-mediated autoimmune diabetes in nonobese diabetic mice. Journal of immunology (Baltimore, Md.: 1950) 161, 3912-3918.
Shao, L., Lie, A. K., Zhang, Y., Wong, C. H., and Kwong, Y. L. (2015). Aberrant germinal center formation, follicular T-helper cells, and germinal center B-cells were involved in chronic graft-versus-host disease. Annals of hematology 94, 1493-1504.
Shen, P., and Fillatreau, S. (2015). Antibody-independent functions of B cells: a focus on cytokines. Nature reviews Immunology 15, 441-451.
Shen, P., Roch, T., Lampropoulou, V., O'Connor, R. A., Stervbo, U., Hilgenberg, E., Ries, S., Dang, V. D., Jaimes, Y., Daridon, C., et al. (2014). IL-35-producing B cells are critical regulators of immunity during autoimmune and infectious diseases. Nature 507, 366-370.
Shi, J., Hou, S., Fang, Q., Liu, X., Liu, X., and Qi, H. (2018). PD-1 Controls Follicular T Helper Cell Positioning and Function. Immunity 49, 264-274.e264.
Shulman, N. R., Marder, V. J., and Weinrach, R. S. (1965). Similarities between known antiplatelet antibodies and the factor responsible for thrombocytopenia in idiopathic purpura. Physiologic, serologic and isotopic studies. Annals of the New York Academy of Sciences 124, 499-542.
Simon, D., Balogh, P., Bognar, A., Kellermayer, Z., Engelmann, P., Nemeth, P., Farkas, N., Minier, T., Lorand, V., Czirjak, L., et al. (2016). Reduced non-switched memory B cell subsets cause imbalance in B cell repertoire in systemic sclerosis. Clinical and experimental rheumatology 34 Suppl 100, 30-36.
Simon, V. M., Parra, A., Minarro, J., Arenas, M. C., Vinader-Caerols, C., and Aguilar, M. A. (2000). Predicting how equipotent doses of chlorpromazine, haloperidol, sulpiride, raclopride and clozapine reduce locomotor activity in mice. European neuropsychopharmacology: the journal of the European College of Neuropsychopharmacology 10, 159-164.
Simpson, N., Gatenby, P. A., Wilson, A., Malik, S., Fulcher, D. A., Tangye, S. G., Manku, H., Vyse, T. J., Roncador, G., Huttley, G. A., et al. (2010). Expansion of circulating T cells resembling follicular helper T cells is a fixed phenotype that identifies a subset of severe systemic lupus erythematosus. Arthritis and rheumatism 62, 234-244.
Singh, I., and Hershman, J. M. (2016). Pathogenesis of Hyperthyroidism. Comprehensive Physiology 7, 67-79.
Smith, J. R., Stempel, A. J., Bharadwaj, A., and Appukuttan, B. (2016). Involvement of B cells in non-infectious uveitis. Clinical & translational immunology 5, e63.
Smith, M. J., Rihanek, M., Coleman, B. M., Gottlieb, P. A., Sarapura, V. D., and Cambier, J. C. (2018). Activation of thyroid antigen-reactive B cells in recent onset autoimmune thyroid disease patients. Journal of autoimmunity 89, 82-89.
Smith, M. J., Simmons, K. M., and Cambier, J. C. (2017). B cells in type 1 diabetes mellitus and diabetic kidney disease. Nature reviews Nephrology 13, 712-720.
Sollid, L. M. (2017). The roles of MHC class II genes and post-translational modification in celiac disease. Immunogenetics 69, 605-616.
Srinivasan, M., Flynn, R., Price, A., Ranger, A., Browning, J. L., Taylor, P. A., Ritz, J., Antin, J. H., Murphy, W. J., Luznik, L., et al. (2012). Donor B-cell alloantibody deposition and germinal center formation are required for the development of murine chronic GVHD and bronchiolitis obliterans. Blood 119, 1570-1580.
Stansky, E., Biancotto, A., Dagur, P. K., Gangaputra, S., Chaigne-Delalande, B., Nussenblatt, R. B., Sen, H. N., and McCoy, J. P., Jr. (2017). B Cell Anomalies in Autoimmune Retinopathy (AIR). Investigative ophthalmology & visual science 58, 3600-3607.
Stoecker, Z. R., George, W. T., O'Brien, J. B., Jancik, J., Colon, E., and Rasimas, J. J. (2017). Clozapine usage increases the incidence of pneumonia compared with risperidone and the general population: a retrospective comparison of clozapine, risperidone, and the general population in a single hospital over 25 months. International clinical psychopharmacology 32, 155-160.
Stohl, W., Hiepe, F., Latinis, K. M., Thomas, M., Scheinberg, M. A., Clarke, A., Aranow, C., Wellborne, F. R., Abud-Mendoza, C., Hough, D. R., et al. (2012). Belimumab reduces autoantibodies, normalizes low complement levels, and reduces select B cell populations in patients with systemic lupus erythematosus. Arthritis and rheumatism 64, 2328-2337.
Stuart, J. M., and Dixon, F. J. (1983). Serum transfer of collagen-induced arthritis in mice. The Journal of experimental medicine 158, 378-392.
Suan, D., Sundling, C., and Brink, R. (2017). Plasma cell and memory B cell differentiation from the germinal center. Current opinion in immunology 45, 97-102.
Suda, T., Chida, K., Hayakawa, H., Imokawa, S., Iwata, M., Nakamura, H., and Sato, A. (1999). Development of bronchus-associated lymphoid tissue in chronic hypersensitivity pneumonitis. Chest 115, 357-363.
Sugimura, T., Shiokawa, S., Haraoka, S., Fujimoto, K., Ohshima, K., Nakamuta, M., and Nishimura, J. (2003). Local antigen-driven oligoclonal expansion of B cells in the liver portal areas of patients with primary biliary cirrhosis. Liver international: official journal of the International Association for the Study of the Liver 23, 323-328.
Sultan, S. M., Ng, K. P., Edwards, J. C., Isenberg, D. A., and Cambridge, G. (2008). Clinical outcome following B cell depletion therapy in eight patients with refractory idiopathic inflammatory myopathy. Clinical and experimental rheumatology 26, 887-893.
Sun, G., Hou, Y., Gong, W., Liu, S., Li, J., Yuan, Y., Zhang, D., Chen, Q., and Yan, X. (2018a). Adoptive Induced Antigen-Specific Treg Cells Reverse Inflammation in Collagen-Induced Arthritis Mouse Model. Inflammation 41, 485-495.
Sun, H., Gao, W., Pan, W., Zhang, Q., Wang, G., Feng, D., Geng, X., Yan, X., and Li, S. (2017). Tim3(+) Foxp3 (+) Treg Cells Are Potent Inhibitors of Effector T Cells and Are Suppressed in Rheumatoid Arthritis. Inflammation 40, 1342-1350.
Sun, W., Meednu, N., Rosenberg, A., Rangel-Moreno, J., Wang, V., Glanzman, J., Owen, T., Zhou, X., Zhang, H., Boyce, B. F., et al. (2018b). B cells inhibit bone formation in rheumatoid arthritis by suppressing osteoblast differentiation. Nature communications 9, 5127.
Suurmond, J., Atisha-Fregoso, Y., Marasco, E., Barley, A. N., Ahmed, N., Calderon, S. A., Wong, M. Y., Mackay, M. C., Aranow, C., and Diamond, B. (2018). Loss of an IgG plasma cell checkpoint in patients with lupus. The Journal of allergy and clinical immunology.
Svensson, L., Jirholt, J., Holmdahl, R., and Jansson, L. (1998). B cell-deficient mice do not develop type II collagen-induced arthritis (CIA). Clinical and experimental immunology 111, 521-526.
Sweet, R. A., Ols, M. L., Cullen, J. L., Milam, A. V., Yagita, H., and Shlomchik, M. J. (2011). Facultative role for T cells in extrafollicular Toll-like receptor-dependent autoreactive B-cell responses in vivo. Proceedings of the National Academy of Sciences of the United States of America 108, 7932-7937.
Szodoray, P., Hajas, A., Kardos, L., Derso, B., Soos, G., Zold, E., Vegh, J., Csipo, I., Nakken, B., Zeher, M., et al. (2012). Distinct phenotypes in mixed connective tissue disease: subgroups and survival. Lupus 21, 1412-1422.
Szodoray, P., Nakken, B., Barath, S., Gaal, J., Aleksza, M., Zeher, M., Sipka, S., Szilagyi, A., Zold, E., Szegedi, G., et al. (2008). Progressive divergent shifts in natural and induced T-regulatory cells signify the transition from undifferentiated to definitive connective tissue disease. International immunology 20, 971-979.
Takahashi, T., Miura, T., Nakamura, J., Yamada, S., Miura, T., Yanagi, M., Matsuda, Y., Usuda, H., Emura, I., Tsuneyama, K., et al. (2012). Plasma cells and the chronic nonsuppurative destructive cholangitis of primary biliary cirrhosis. Hepatology (Baltimore, Md.) 55, 846-855.
Tamimoto, Y., Horiuchi, T., Tsukamoto, H., Otsuka, J., Mitoma, H., Kimoto, Y., Nakashima, H., Muta, K., Abe, Y., Kiyohara, C., et al. (2008). A dose-escalation study of rituximab for treatment of systemic lupus erythematosus and Evans' syndrome: immunological analysis of B cells, T cells and cytokines. Rheumatology (Oxford, England) 47, 821-827.
Tangye, S. G., Brink, R., Goodnow, C. C., and Phan, T. G. (2015). SnapShot: Interactions between B Cells and T Cells. Cell 162, 926-926.e921.
Tani, C., Carli, L., Vagnani, S., Talarico, R., Baldini, C., Mosca, M., and Bombardieri, S. (2014). The diagnosis and classification of mixed connective tissue disease. Journal of autoimmunity 48-49, 46-49.
Tanyeri, M. H., Buyukokuroglu, M. E., Tanyeri, P., Mutlu, O., Akar, F. Y., Ulak, G., and Erden, B. F. (2017). Effects of long-term treatment with haloperidol, clozapine and aripiprazole on mice isolated vas deferens. International urology and nephrology 49, 1561-1567.
Taylor, D. K., Mittereder, N., Kuta, E., Delaney, T., Burwell, T., Dacosta, K., Zhao, W., Cheng, L. I., Brown, C., Boutrin, A., et al. (2018). T follicular helper-like cells contribute to skin fibrosis. Science translational medicine 10.
Tedder, T. F., Poe, J. C., Fujimoto, M., Haas, K. M., and Sato, S. (2005). The CD19-CD21 signal transduction complex of B lymphocytes regulates the balance between health and autoimmune disease: systemic sclerosis as a model system. Current directions in autoimmunity 8, 55-90.
Tellier, J., and Nutt, S. L. (2018). Plasma cells: the programming of an antibody-secreting machine. European journal of immunology.
Tembhre, M. K., Parihar, A. S., Sharma, V. K., Sharma, A., Chattopadhyay, P., and Gupta, S. (2015). Alteration in regulatory T cells and programmed cell death 1-expressing regulatory T cells in active generalized vitiligo and their clinical correlation. The British journal of dermatology 172, 940-950.
Thomas, L., Canoui-Poitrine, F., Gottenberg, J. E., Economu-Dubosc, A., Medkour, F., Chevalier, X., Bastuji-Garin, S., Le Louet, H., Farrenq, V., and Claudepierre, P. (2012). Incidence of new-onset and flare of preexisting psoriasis during rituximab therapy for rheumatoid arthritis: data from the French AIR registry. The Journal of rheumatology 39, 893-898.
Timmermans, W. M., van Laar, J. A., van der Houwen, T. B., Kamphuis, L. S., Bartol, S. J., Lam, K. H., Ouwendijk, R. J., Sparrow, M. P., Gibson, P. R., van Hagen, P. M., et al. (2016). B-Cell Dysregulation in Crohn's Disease Is Partially Restored with Infliximab Therapy. PloS one 11, e0160103.
Tokunaga, M., Saito, K., Kawabata, D., Imura, Y., Fujii, T., Nakayamada, S., Tsujimura, S., Nawata, M., Iwata, S., Azuma, T., et al. (2007). Efficacy of rituximab (anti-CD20) for refractory systemic lupus erythematosus involving the central nervous system. Annals of the rheumatic diseases 66, 470-475.
Trouw, L. A., Haisma, E. M., Levarht, E. W., van der Woude, D., Loan-Facsinay, A., Daha, M. R., Huizinga, T. W., and Toes, R. E. (2009). Anti-cyclic citrullinated peptide antibodies from rheumatoid arthritis patients activate complement via both the classical and alternative pathways. Arthritis and rheumatism 60, 1923-1931.
Tsoi, M. S., Storb, R., Jones, E., Weiden, P. L., Shulman, H., Witherspoon, R., Atkinson, K., and Thomas, E. D. (1978). Deposition of IgM and complement at the dermoepidermal junction in acute and chronic cutaneous graft-vs-host disease in man. Journal of immunology (Baltimore, Md.: 1950) 120, 1485-1492.
Tsubakio, T., Kurata, Y., Kanayama, Y., Yonezawa, T., Tarui, S., and Kitani, T. (1983). In vitro platelet phagocytosis in idiopathic thrombocytopenic purpura. Acta haematologica 70, 250-256.
Tsuda, M., Moritoki, Y., Lian, Z.-X., Zhang, W., Yoshida, K., Wakabayashi, K., Yang, G.-X., Nakatani, T., Vierling, J., Lindor, K., et al. (2012). Biochemical and immunologic effects of rituximab in patients with primary biliary cirrhosis and an incomplete response to ursodeoxycholic acid. Hepatology (Baltimore, Md.) 55, 512-521.
Tsuneyama, K., Baba, H., Morimoto, Y., Tsunematsu, T., and Ogawa, H. (2017). Primary Biliary Cholangitis: Its Pathological Characteristics and Immunopathological Mechanisms. The journal of medical investigation: JMI 64, 7-13.
Tzartos, J. S., Craner, M. J., Friese, M. A., Jakobsen, K. B., Newcombe, J., Esiri, M. M., and Fugger, L. (2011). IL-21 and IL-21 receptor expression in lymphocytes and neurons in multiple sclerosis brain. The American journal of pathology 178, 794-802.
Ueki, I., Abiru, N., Kobayashi, M., Nakahara, M., Ichikawa, T., Eguchi, K., and Nagayama, Y. (2011). B cell-targeted therapy with anti-CD20 monoclonal antibody in a mouse model of Graves' hyperthyroidism. Clinical and experimental immunology 163, 309-317.
Uzzan, M., Ko, H. M., Rosenstein, A. K., Pourmand, K., Colombel, J. F., and Mehandru, S. (2018). Efficient long-term depletion of CD20(+) B cells by rituximab does not affect gut-resident plasma cells. Annals of the New York Academy of Sciences 1415, 5-10.
van de Wal, Y., Kooy, Y. M., van Veelen, P. A., Pena, S. A., Mearin, L. M., Molberg, O., Lundin, K. E., Sollid, L. M., Mutis, T., Benckhuijsen, W. E., et al. (1998). Small intestinal T cells of celiac disease patients recognize a natural pepsin fragment of gliadin. Proceedings of the National Academy of Sciences of the United States of America 95, 10050-10054.
Van der Weerd, K., Van Hagen, P. M., Schrijver, B., Kwekkeboom, D. J., De Herder, W. W., Ten Broek, M. R., Postema, P. T., Van Dongen, J. J., Staal, F. J., and Dik, W. A. (2013). The peripheral blood compartment in patients with Graves' disease: activated T lymphocytes and increased transitional and pre-naive mature B lymphocytes. Clinical and experimental immunology 174, 256-264.
van Schaik, F. D., Oldenburg, B., Hart, A. R., Siersema, P. D., Lindgren, S., Grip, O., Teucher, B., Kaaks, R., Bergmann, M. M., Boeing, H., et al. (2013). Serological markers predict inflammatory bowel disease years before the diagnosis. Gut 62, 683-688.
Vancsa, A., Szabo, Z., Szamosi, S., Bodnar, N., Vegh, E., Gergely, L., Szucs, G., Szanto, S., and Szekanecz, Z. (2013). Longterm effects of rituximab on B cell counts and autoantibody production in rheumatoid arthritis: use of high-sensitivity flow cytometry for more sensitive assessment of B cell depletion. The Journal of rheumatology 40, 565-571.
Vannucchi, G., Covelli, D., Curro, N., Dazzi, D., Maffini, A., Campi, I., Bonara, P., Guastella, C., Pignataro, L., Ratiglia, R., et al. (2012). Serum BAFF concentrations in patients with Graves' disease and orbitopathy before and after immunosuppressive therapy. The Journal of clinical endocrinology and metabolism 97, E755-759.
Verburg, R. J., Flierman, R., Sont, J. K., Ponchel, F., van Dreunen, L., Levarht, E. W., Welling, M. M., Toes, R. E., Isaacs, J. D., and van Laar, J. M. (2005). Outcome of intensive immunosuppression and autologous stem cell transplantation in patients with severe rheumatoid arthritis is associated with the composition of synovial T cell infiltration. Annals of the rheumatic diseases 64, 1397-1405.
Verstappen, G. M., Kroese, F. G., Meiners, P. M., Corneth, O. B., Huitema, M. G., Haacke, E. A., van der Vegt, B., Arends, S., Vissink, A., Bootsma, H., et al. (2017). B Cell Depletion Therapy Normalizes Circulating Follicular Th Cells in Primary Sjogren Syndrome. The Journal of rheumatology 44, 49-58.
Vieira, P., and Rajewsky, K. (1988). The half-lives of serum immunoglobulins in adult mice. European journal of immunology 18, 313-316.
Viisanen, T., Ihantola, E. L., Nanto-Salonen, K., Hyoty, H., Nurminen, N., Selvenius, J., Juutilainen, A., Moilanen, L., Pihlajamaki, J., Veijola, R., et al. (2017). Circulating CXCR5+PD-1+ICOS+ Follicular T Helper Cells Are Increased Close to the Diagnosis of Type 1 Diabetes in Children With Multiple Autoantibodies. Diabetes 66, 437-447.
Vital, E. M., Dass, S., Buch, M. H., Henshaw, K., Pease, C. T., Martin, M. F., Ponchel, F., Rawstron, A. C., and Emery, P. (2011). B cell biomarkers of rituximab responses in systemic lupus erythematosus. Arthritis and rheumatism 63, 3038-3047.
Vuga, L. J., Tedrow, J. R., Pandit, K. V., Tan, J., Kass, D. J., Xue, J., Chandra, D., Leader, J. K., Gibson, K. F., Kaminski, N., et al. (2014). C-X-C motif chemokine 13 (CXCL13) is a prognostic biomarker of idiopathic pulmonary fibrosis. American journal of respiratory and critical care medicine 189, 966-974.
Wallace, Z. S., Mattoo, H., Carruthers, M., Mahajan, V. S., Della Torre, E., Lee, H., Kulikova, M., Deshpande, V., Pillai, S., and Stone, J. H. (2015). Plasmablasts as a biomarker for IgG4-related disease, independent of serum IgG4 concentrations. Annals of the rheumatic diseases 74, 190-195.
Wallis, A., and Spinks, K. (2015). The diagnosis and management of interstitial lung diseases. BMJ (Clinical research ed) 350, h2072.
Wang, L., Sun, X., Qiu, J., Cai, Y., Ma, L., Zhao, P., and Jiang, Y. (2015). Increased numbers of circulating ICOS(+) follicular helper T and CD38(+) plasma cells in patients with newly diagnosed primary biliary cirrhosis. Digestive diseases and sciences 60, 405-413.
Wang, R. X., Yu, C. R., Dambuza, I. M., Mandi, R. M., Dolinska, M. B., Sergeev, Y. V., Wingfield, P. T., Kim, S. H., and Egwuagu, C. E. (2014a). Interleukin-35 induces regulatory B cells that suppress autoimmune disease. Nature medicine 20, 633-641.
Wang, X., Jiang, Y., Zhu, Y., Zhang, M., Li, M., Wang, H., and Gao, P. (2016a). Circulating memory B cells and plasmablasts are associated with the levels of serum immunoglobulin in patients with ulcerative colitis. Journal of cellular and molecular medicine 20, 804-814.
Wang, X., Zhu, Y., Zhang, M., Hou, J., Wang, H., Jiang, Y., Wang, H., and Gao, P. (2017). The shifted balance between circulating follicular regulatory T cells and follicular helper T cells in patients with ulcerative colitis. Clinical science (London, England: 1979) 131, 2933-2945.
Wang, Y., Wang, L., Yang, H., Yuan, W., Ren, J., and Bai, Y. (2016b). Activated Circulating T Follicular Helper Cells Are Associated with Disease Severity in Patients with Psoriasis. Journal of immunology research 2016, 7346030.
Wang, Z., Wang, Z., Diao, Y., Qian, X., Zhu, N., and Dong, W. (2014b). Circulating follicular helper T cells in Crohn's disease (CD) and CD-associated colorectal cancer. Tumour biology: the journal of the International Society for Oncodevelopmental Biology and Medicine 35, 9355-9359.
Wangel, A. G., Johansson, E., and Ranki, A. (1984). Polyclonal B-cell activation and increased lymphocyte helper-suppressor ratios in discoid lupus erythematosus. The British journal of dermatology 110, 665-669.
Wehr, C., Kivioja, T., Schmitt, C., Ferry, B., Witte, T., Eren, E., Vlkova, M., Hernandez, M., Detkova, D., Bos, P. R., et al. (2008). The EUROclass trial: defining subgroups in common variable immunodeficiency. Blood 111, 77-85.
Wei, Y., and Hou, M. (2016). T cells in the pathogenesis of immune thrombocytopenia. Seminars in hematology 53 Suppl 1, S13-15.
Williams, A. A., Ingram, W. M., Levine, S., Resnik, J., Kamel, C. M., Lish, J. R., Elizalde, D. I., Janowski, S. A., Shoker, J., Kozlenkov, A., et al. (2012). Reduced levels of serotonin 2A receptors underlie resistance of Egr3-deficient mice to locomotor suppression by clozapine. Neuropsychopharmacology: official publication of the American College of Neuropsychopharmacology 37, 2285-2298.
Williams, R. O. (2012). What Have We Learned about the Pathogenesis of Rheumatoid Arthritis from TNF-Targeted Therapy? ISRN Immunology 2012, 15.
Wing, J. B., Tekguc, M., and Sakaguchi, S. (2018). Control of Germinal Center Responses by T-Follicular Regulatory Cells. Frontiers in immunology 9, 1910.
Winqvist, O., Karlsson, F. A., and Kampe, O. (1992). 21-Hydroxylase, a major autoantigen in idiopathic Addison's disease. Lancet (London, England) 339, 1559-1562.
Wouters, C. H., Diegenant, C., Ceuppens, J. L., Degreef, H., and Stevens, E. A. (2004). The circulating lymphocyte profiles in patients with discoid lupus erythematosus and systemic lupus erythematosus suggest a pathogenetic relationship. The British journal of dermatology 150, 693-700.
Xu, H., Li, X., Liu, D., Li, J., Zhang, X., Chen, X., Hou, S., Peng, L., Xu, C., Liu, W., et al. (2013). Follicular T-helper cell recruitment governed by bystander B cells and ICOS-driven motility. Nature 496, 523-527.
Xue, J., Kass, D. J., Bon, J., Vuga, L., Tan, J., Csizmadia, E., Otterbein, L., Soejima, M., Levesque, M. C., Gibson, K. F., et al. (2013). Plasma B lymphocyte stimulator and B cell differentiation in idiopathic pulmonary fibrosis patients. Journal of immunology (Baltimore, Md.: 1950) 191, 2089-2095.
Yanaba, K., Hamaguchi, Y., Venturi, G. M., Steeber, D. A., St Clair, E. W., and Tedder, T. F. (2007). B cell depletion delays collagen-induced arthritis in mice: arthritis induction requires synergy between humoral and cell-mediated immunity. Journal of immunology (Baltimore, Md.: 1950) 179, 1369-1380.
Yoshitomi, H., Kobayashi, S., Miyagawa-Hayashino, A., Okahata, A., Doi, K., Nishitani, K., Murata, K., Ito, H., Tsuruyama, T., Haga, H., et al. (2018). Human Sox4 facilitates the development of CXCL13-producing helper T cells in inflammatory environments. Nature communications 9, 3762.
Yoshizaki, A., Miyagaki, T., DiLillo, D. J., Matsushita, T., Horikawa, M., Kountikov, E. I., Spolski, R., Poe, J. C., Leonard, W. J., and Tedder, T. F. (2012). Regulatory B cells control T-cell autoimmunity through IL-21-dependent cognate interactions. Nature 491, 264-268.
Yuan, Y., Qiu, J., Lin, Z. T., Li, W., Haley, C., Mui, U. N., Ning, J., Tyring, S. K., and Wu, T. (2019). Identification of Novel Autoantibodies Associated with Psoriatic Arthritis. Arthritis & rheumatology (Hoboken, N.J.).
Yurasov, S., Wardemann, H., Hammersen, J., Tsuiji, M., Meffre, E., Pascual, V., and Nussenzweig, M. C. (2005). Defective B cell tolerance checkpoints in systemic lupus erythematosus. The Journal of experimental medicine 201, 703-711.
Zanoni, G., Navone, R., Lunardi, C., Tridente, G., Bason, C., Sivori, S., Beri, R., Dolcino, M., Valletta, E., Corrocher, R., et al. (2006). In celiac disease, a subset of autoantibodies against transglutaminase binds toll-like receptor 4 and induces activation of monocytes. PLoS medicine 3, e358.
Zehentmeier, S., Roth, K., Cseresnyes, Z., Sercan, O., Horn, K., Niesner, R. A., Chang, H. D., Radbruch, A., and Hauser, A. E. (2014). Static and dynamic components synergize to form a stable survival niche for bone marrow plasma cells. European journal of immunology 44, 2306-2317.
Zeiser, R., and Blazar, B. R. (2017). Acute Graft-versus-Host Disease—Biologic Process, Prevention, and Therapy. The New England journal of medicine 377, 2167-2179.
Zha, B., Huang, X., Lin, J., Liu, J., Hou, Y., and Wu, G. (2014). Distribution of lymphocyte subpopulations in thyroid glands of human autoimmune thyroid disease. Journal of clinical laboratory analysis 28, 249-254.
Zhang, J., Zhang, W., Leung, P. S., Bowlus, C. L., Dhaliwal, S., Coppel, R. L., Ansari, A. A., Yang, G. X., Wang, J., Kenny, T. P., et al. (2014). Ongoing activation of autoantigen-specific B cells in primary biliary cirrhosis. Hepatology (Baltimore, Md.) 60, 1708-1716.
Zhang, X., Lindwall, E., Gauthier, C., Lyman, J., Spencer, N., Alarakhia, A., Fraser, A., Ing, S., Chen, M., Webb-Detiege, T., et al. (2015). Circulating CXCR5+CD4+ helper T cells in systemic lupus erythematosus patients share phenotypic properties with germinal center follicular helper T cells and promote antibody production. Lupus 24, 909-917.
Zhang, Y., Tech, L., George, L. A., Acs, A., Durrett, R. E., Hess, H., Walker, L. S. K., Tarlinton, D. M., Fletcher, A. L., Hauser, A. E., et al. (2018). Plasma cell output from germinal centers is regulated by signals from Tfh and stromal cells. The Journal of experimental medicine 215, 1227-1243.
Zhou, Z., Niu, H., Zheng, Y. Y., and Morel, L. (2011). Autoreactive marginal zone B cells enter the follicles and interact with CD4⁺ T cells in lupus-prone mice. BMC immunology 12, 7.
Zhu, C., Ma, J., Liu, Y., Tong, J., Tian, J., Chen, J., Tang, X., Xu, H., Lu, L., and Wang, S. (2012). Increased frequency of follicular helper T cells in patients with autoimmune thyroid disease. The Journal of clinical endocrinology and metabolism 97, 943-950.
Ziegler, A. G., Rewers, M., Simell, O., Simell, T., Lempainen, J., Steck, A., Winkler, C., Ilonen, J., Veijola, R., Knip, M., et al. (2013). Seroconversion to multiple islet autoantibodies and risk of progression to diabetes in children. Jama 309, 2473-2479.
Zotos, D., Coquet, J. M., Zhang, Y., Light, A., D'Costa, K., Kallies, A., Corcoran, L. M., Godfrey, D. I., Toellner, K. M., Smyth, M. J., et al. (2010). IL-21 regulates germinal center B cell differentiation and proliferation through a B cell-intrinsic mechanism. The Journal of experimental medicine 207, 365-378.

1. (canceled)

2. A method of treatment or prevention of a pathogenic immunoglobulin driven B cell disease with a T cell component in a subject by administering to said subject an effective amount of a compound selected from clozapine, norclozapine and prodrugs thereof and pharmaceutically acceptable salts and solvates thereof wherein said compound causes mature B cells to be inhibited in said subject.

3. (canceled)

4. The method according to claim 2 to 3 wherein the compound is clozapine or a pharmaceutically acceptable salt or solvate thereof.

5. The method according to claim 2 to 4 wherein the mature B cells are class switched memory B cells.

6. The method according to claim 2 to 4 wherein the mature B cells are plasmablasts.

7. The method according to claim 2 wherein the pathogenic immunoglobulin driven B cell disease with a T cell component is a disease selected from the group consisting of vitiligo, psoriasis, coeliac disease, dermatitis herpetiformis, discoid lupus erythematosus, dermatomyositis, polymyositis, Type 1 diabetes mellitus, autoimmune Addison's disease, multiple sclerosis, interstitial lung disease, Crohn's disease, ulcerative colitis, thyroid autoimmune disease, autoimmune uveitis, primary biliary cirrhosis, primary sclerosing cholangitis, undifferentiated connective tissue disease, autoimmune thrombocytopenic purpura, mixed connective tissue disease, an immune-mediated inflammatory disease (IMID) such as scleroderma, rheumatoid arthritis, Sjogren's disease, and an autoimmune connective tissue disease such as systemic lupus erythematosus.

8. The method according to claim 7 wherein the pathogenic immunoglobulin driven B cell disease with a T cell component is psoriasis, a connective tissue disease such as systemic lupus erythematosus, or an immune-mediated inflammatory disease (IMID) such as scleroderma, rheumatoid arthritis or Sjogren's disease.

9. The method according to claim 2 wherein the pathogenic immunoglobulin driven B cell disease with a T cell component is graft versus host disease.

10. The method according claim 2 wherein the compound has the effect of decreasing CD19 (+) B cells and/or (−) B-plasma cells.

11. A method of treatment or prevention of a pathogenic immunoglobulin driven B cell disease with a T cell component in a subject comprising administering to the subject an effective amount of a pharmaceutical composition comprising a compound selected from clozapine, norclozapine and prodrugs thereof and pharmaceutically acceptable salts and solvates thereof; and a pharmaceutically acceptable diluent or carrier, wherein said compound causes mature B cells to be inhibited in said subject.

12. The method according to claim 11 wherein the pharmaceutical composition is administered orally.

13. The method according to claim 11 wherein the pharmaceutical composition is formulated as a liquid or solid, such as a syrup, suspension, emulsion, tablets, capsule or lozenge.

14. The method according to claim 11 wherein the mature B cells are class switched memory B cells.

15. The method according to claim 11 wherein the mature B cells are plasmablasts.

16. A method according to claim 2 wherein the compound is administered in combination with a second or further therapeutic agent for the treatment or prevention of a pathogenic immunoglobulin driven B cell disease with a T cell component.

17. The method according to claim 16 wherein the second or further substance for the treatment or prevention of a pathogenic immunoglobulin driven B cell disease with a T cell component is selected from anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or azathioprine), general anti-inflammatories (such as hydroxychloroquine or NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab).
